(19) United States
(12) Patent Application Publication
(10) Pub. No.: US 2025/0179580 A1
(43) Pub. Date: Jun. 5, 2025

(54) METHODS OF DETERMINING QUANTITATIVE METHYLATION DATA

(71) Applicant: University of Utah Research Foundation, Salt Lake City, UT (US)

(72) Inventors: Sabine Hellwig, Salt Lake City, UT (US); Devin Close, Salt Lake City, UT (US); Amy Farrington, Salt Lake City, UT (US); Anna Matynia, Salt Lake City, UT (US)

(21) Appl. No.: 18/742,601

(22) Filed: Jun. 13, 2024

Related U.S. Application Data
(60) Provisional application No. 63/606,418, filed on Dec. 5, 2023.

(51) Int. Cl. C12Q 1/6886; A61K 31/495

(52) U.S. Cl. CPC C12Q 1/6886; A61K 31/495; C12Q 2600/154

(57) ABSTRACT

Disclosed are methods of determining methylation status of a target nucleic acid sequence in a sample comprising determining quantitative methylation data at two or more predetermined methylation sites in the target nucleic acid sequence, wherein the target nucleic acid sequence comprises an O6-methylguanine-DNA-methyltransferase (MGMT) promoter. Disclosed are methods of treating a subject having cancer comprising determining, from a sample, quantitative methylation data at two or more predetermined methylation sites in a MGMT promoter, wherein the sample is from the subject having cancer; and treating the subject for cancer when the quantitative methylation data at two or more predetermined methylation sites is at or above a threshold.

### CROSS-REFERENCE TO RELATED APPLICATIONS

This application claims the benefit of U.S. Provisional Patent Application No. 63/606,418, filed Dec. 5, 2023, which is incorporated by reference herein in its entirety.

### REFERENCE TO SEQUENCE LISTING

The Sequence Listing submitted Sep. 30, 2024 as a text file named “21101.0472U2.xml,” created on Aug. 15, 2024, and having a size of 8,125 bytes is hereby incorporated by reference pursuant to 37 C.F.R. § 1.52(e)(5).

### BACKGROUND

MGMT methylation is present in several types of human cancer and is considered a prognostic biomarker in glioblastoma. The O6-methylguanine-DNA-methyltransferase (MGMT) gene encodes for an important DNA repair protein which acts by removing alkyl products from the O6 position on guanine. A so-called “suicide enzyme,” following removal of the alkyl groups, the newly alkylated MGMT protein, is then marked for degradation by ubiquitinization. Proper functioning of the gene is important for maintaining cell integrity. Epigenetic silencing of the MGMT gene by methylation of the CpG islands of the promoter region has been shown to correlate with loss of gene transcription and protein expression. Loss of expression of the MGMT protein results in decreased DNA repair and retention of alkyl groups, thereby allowing alkylating agents such as carmustine (BCNU), lomustine (CCNU), and temozolomide to have greater efficacy in patients whose tumors exhibit hypermethylation of the MGMT promoter and reducing the MGMT protein concentration. Although MGMT protein expression is expressed in a wide variety of tumors including colon, head and neck, and lung cancers, infiltrative gliomas remain one of the most intriguing and potentially informative tumor groups to study the impact of MGMT expression.

### BRIEF SUMMARY

Disclosed are methods of determining methylation status of a target nucleic acid sequence in a sample comprising determining quantitative methylation data at two or more predetermined methylation sites in the target nucleic acid sequence, wherein the target nucleic acid sequence comprises an O6-methylguanine-DNA-methyltransferase (MGMT) promoter.

Disclosed are methods of treating a subject having cancer comprising determining, from a sample, quantitative methylation data at two or more predetermined methylation sites in a MGMT promoter, wherein the sample is from the subject having cancer; and treating the subject for cancer when the quantitative methylation data at two or more predetermined methylation sites is at or above a threshold.

Disclosed are methods of assessing survival probability in a subject having cancer comprising determining, from a sample, quantitative methylation data at two or more predetermined methylation sites in a target nucleic acid sequence, wherein the sample is from the subject having cancer, wherein the target nucleic acid sequence comprises a MGMT promoter, wherein quantitative methylation data below a threshold indicates decreased survival probability.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a schematic of the MGMT locus. The location of the full MGMT promoter CpG island (bottom bar) in relation to exon 1 are shown. The region of interest, encompassing the CpGs relevant to this validation is enhanced and CpG coverage by assays discussed in this validation is indicated.

FIGS. 2A-2C show a graphic overview of MGMT ddPCR primer and probe locations. FIG. 2A shows a native target sequence. FIG. 2B shows a methylated sequence and unmethylated sequence after bisulfite conversion. FIG. 2C shows an unmethylated-specific probe and methylation-specific probe.

FIG. 3 shows an example split-plate layout used for the MGMT ddPCR test. Positions of controls (POS=fully methylated, LOW=10% methylated, NEG=nonmethylated, NTC=no template control) are locked. Up to 44 patient samples (P1-44) can be tested per run.

FIGS. 4A and 4B show an appearance of two-dimensional ddPCR data plots. (FIG. 4A) Generic two-dimensional raw data plot obtained for duplex assays employing differentially labeled allele-specific probes. The mapping of allele copies to the four analysis sectors is indicated. (FIG. 4B) Sample data plots for a methylation positive patient sample. Operator-applied gates are shown in magenta with the FAM+/HEX− sector droplet counts indicated on the plot. Droplet counts (total accepted, FAM-positive:HEX-positive, calculated resulting allele copy numbers (M=Methylated; NM=Nonmethylated), qualitative call and percent detected methylation are annotated below each plot.

FIG. 5 shows an example data interpretation schematic used for validation.

FIG. 6 shows cluster mapping of fully and partially methylated allele copies as determined during feasibility studies. The approximate plot positions of droplets containing amplified copies of synthetic templates with the indicated fraction of sites methylated is indicated.

FIGS. 7A-7D show differences in raw data interpretation to determine percent methylation by different methodologies. Sample raw data plots for the CpG79-82 region of MGMT_011 obtained by MassARRAY (FIG. 7A), pyrosequencing (FIG. 7B) and ddPCR (FIG. 7C) is shown. For MassARRAY quantitative data is obtained by integrating the areas under the curve of peaks that correspond to alleles with 0, 1, 2 or 3 methylated sites. For pyrosequencing, which allows for high resolution of data at single CpG sites, the peak heights relating unmethylated and methylated cytosines at each position are compared. ddPCR yields data that allocates discreet quanta of 100%, 50% or 0% methylation to individual raw data points (droplets) on the plot. Quantitative data for the total reaction is obtained from an average of all data points. A simplified model in (FIG. 7D) illustrates how the incremental or quantum-valued interpretation at the level of individual allele copy can result in differences in mean detected percent methylation in certain samples. Shown are two samples consisting of four allele copies each that have an apparent overall methylation level of 25% (4 of 16 sites methylated).

FIG. 8 shows a comparison of MGMT methylation levels determined by ddPCR and pyrosequencing (PSQ) for sample MSIN_039. Vertical dashed lines indicate the mean percent methylation detected over the CpG sites covered by each test methodology.

FIG. 9 shows raw FAM-only droplet counts observed as a function of total amplifiable MGMT copy number in both assay wells.

FIGS. 10A and 10B show: FIG. 10A. At value percent MGMT methylation measured by ddPCR in MSI normal FFPE patient samples with raw droplet counts scoring within (black circles) or above (magenta circles) assay noise. FIG. 10B. At value percent MGMT methylation measured by ddPCR and pyrosequencing (PSQ) for a subset of samples shown in A (15 within noise, 8 above). ddPCR and PSQ methylation levels detected in replicates of a nonmethylated standard (Zymo) are included for reference.

FIG. 11 shows tissue of origin of presumed normal FFPE samples used for specificity testing of MGMT methylation by ddPCR.

FIG. 12 shows a table with MGMT methylation levels as detected by ddPCR in a cohort of MSI normal specificity samples. Samples that were excluded from the cohort due to quality control failure are listed at the bottom of the table, numbers 53-59.

FIG. 13 is a table with overview of accuracy sample demographics

FIG. 14 is a table showing MassARRAY and ddPCR MGMT methylation detection thresholds binned by observed % methylation. The thresholding used for Pyrosequencing performed as part of the validation is included for reference

FIG. 15 shows quantitative correlation of observed percent methylation by MassARRAY (MA) and ddPCR in a cohort of 50 patient samples with Detected MassARRAY clinical testing results. The linear regression line (solid black) and line of identity (diagonal dashed line) as well as qualitative call thresholds are indicated. Data points for samples with Low Level methylation by ddPCR are shown between the two horizontal dashed lines.

FIG. 16 shows observed percent methylation by ddPCR in a cohort of 52 patient samples with Not Detected reported clinical outcomes by MassARRAY. Samples are grouped into subsets that showed raw FAM-only droplet counts within the assay noise range (<5) in both wells (dark circles) or with above noise counts in at least one test well (lighter circles). The presumed clinically relevant reporting threshold of 5% methylation applied in this validation is shown for reference.

FIG. 17 shows a correlation of percent methylation in a cohort of patient samples with Not Detected outcomes as determined by MassARRAY and ddPCR testing. ddPCR methylation is stated “at value”, taking all detected FAM+ droplet counts (above and within presumed assay noise) into account.

FIG. 18 shows a table with results of ddPCR testing of 50 samples resulted as Detected by MassARRAY.

FIG. 19 shows a table with results of ddPCR testing of 53 patient samples resulted as Not Detected by MassARRAY.

FIG. 20 shows a table with results of ddPCR testing of 59 patient samples resulted as Low Level by MassARRAY.

FIG. 21 shows a table with CpG coverage consolidation and third-method testing of discordant accuracy samples. For samples that had clinical results reported as Low Level by MassARRAY but showed not detected outcomes by ddPCR the matching de-identified clinical report was interrogated. To limit the CpG coverage of the MassARRAY assay to match the CpG coverage of the ddPCR (75-82) and Pyrosequencing assay (74-83) more closely, quantitative data was obtained by excluding the “SAR” region alone or excluding “SAR” and CpG group 72-73. Because the pyrosequencing assay allows for single-CpG data resolution, quantitative data for CpGs 75-82 were examined in addition to the full CpG coverage of the assay (CpGs 74-83).

FIG. 22 shows a table with qualitative concordance tabulation for 23 discordant Low Level MassARRAY samples. * indicates that concordance required restricting pyrosequencing coverage to the same CpG region covered by ddPCR (75-81)

FIGS. 23A and 23B show assay quantitative baseline observed by testing a commercial nonmethylated standard (A) and ten concordant Not Detected patient samples by MassARRAY, pyrosequencing (PSQ) and ddPCR. The range of CpG covered is indicated. For MassARRAY data, control reports from ten random clinical runs were queried. PSQ data was obtained from seven experiments and ddPCR data was collected from all 21 validation runs.

FIG. 24 shows a table with concordance overview of MGMT methylation status obtained by ddPCR testing in comparison to clinical test outcomes by MassARRAY. Data from a cohort of 137 qualifying patient samples is summarized. * indicates concordant sample numbers. Discordant outcomes are **.

FIG. 25 shows a table with an overview of dilution series prepared for validation of assay linearity.

FIG. 26 shows a table of a goodness of fit summary for linearity validation. All of the R2 values are above the validation threshold of 0.95.

FIG. 27A-27F shows observed percent methylation as a function of calculated expected values for dilution series generated for validation of assay linearity. The percent methylation for CpGs 75-78 (left panels), CpGs 79-82 (center panels) and the mean of both assay wells (right panels) is shown. Lower panels display the data points below 20% methylation only. The gray shaded area represents the methylation range below the validated assay LOD of 5%. For series tested in triplicates individual replicates are shown. Green circles represent data points above the assay noise, red data points showed raw droplet counts with the assay noise. For mean data green data points indicate both wells scored above assay noise. The trendline for a linear regression and the line of identity are shown.

FIG. 28 shows a table with a goodness of fit of data points above and below the LOD of 5% methylation in the linearity dilution series. The validation threshold is 0.95.

FIGS. 29A-29F show tables summarizing quantitative and qualitative outcomes for all samples included in the dilution series studies. FIG. 29A is a Summary table for the 163_N006 dilution series as analyzed to validate the assay LOD of 5% mean methylation. Raw droplet counts in the FAM+/HEX− gating quadrant, detected percent methylation at value and qualitative results are shown. FIG. 29B: Summary table for the 169_N009 dilution series as analyzed to validate the assay LOD of 5% mean methylation. Raw droplet counts in the FAM+/HEX− gating quadrant, detected percent methylation at value and qualitative results are shown. FIG. 29C: Summary table for the 170_NP dilution series as analyzed to validate the assay LOD of 5% mean methylation. Raw droplet counts in the FAM+/HEX− gating quadrant, detected percent methylation at value and qualitative results are shown. FIG. 29D: Summary table for the 142_097 dilution series as analyzed to validate the assay LOD of 5% mean methylation. Raw droplet counts in the FAM+/HEX− gating quadrant, detected percent methylation at value and qualitative results are shown. FIG. 29E: Summary table for the Zymo Standards dilution series as analyzed to validate the assay LOD of 5% mean methylation. Raw droplet counts in the FAM+/HEX− gating quadrant, detected percent methylation at value and qualitative results are shown. FIG. 29F: Summary table for the 135_N009 dilution series as analyzed to validate the assay LOD of 5% mean methylation. Raw droplet counts in the FAM+/HEX− gating quadrant, detected percent methylation at value and qualitative results are shown.

FIGS. 30A and 30B show tables that summarize precision and accuracy observed in the dilution series generated to inform the LOQ of the assay. FIG. 30A: LOQ estimation for the Zymo Standard dilution series (mean total converted copies/well: 483). % CV values≤30 are highlighted in green. Values that fail this metric are shown in red. FIG. 30B: LOQ estimation for the 135_N009 patient sample dilution series (mean total converted copies/well: 1740).

FIG. 31 shows a graphic representation of % CV Precision (blue circles) and % CV Accuracy (purple squares) observed in LOQ test series. The horizontal dashed line indicates the cutoff level of 30% used for the purpose of this validation.

FIGS. 32A and 32B quantitative and qualitative outcomes for all samples included in the within-run precision studies. FIG. 32A: Within-run precision studies for a sample cohort with Not Detected outcomes by MassARRAY. FIG. 32B: Within-run precision studies for a sample cohort with Detected or Low Level methylation outcomes by MassARRAY.

FIGS. 33A-33C show quantitative and qualitative outcomes for all samples included in the between-run and between-operator precision studies. FIG. 33A: Between-run precision studies for MSI normal specificity samples and a cohort with Not Detected outcomes by MassARRAY. FIG. 33B: Between-run precision studies for MSI normal specificity samples and a cohort with Detected outcomes by MassARRAY. FIG. 33C: Between-run precision studies for MSI normal specificity samples and a cohort with Low Level outcomes by MassARRAY. Discordant results are highlighted in red.

FIG. 34 shows data plots of qualitatively discordant samples in between-run precision validation.

FIG. 35 shows a table with an overview of qualitative and quantitative agreement observed in within-run reproducibility studies.

FIG. 36 shows a table with an overview of qualitative and quantitative agreement observed in between-run reproducibility studies.

FIGS. 37A and 37B show FIG. 37A. Consistency in total detected MGMT allele copies is tracked as a function of storage time of the crude FFPE extract under refrigerated conditions. Relative numbers in each well when compared to Day 0 measurements are shown for each individual sample as well as a mean change±standard deviation of all 11 extracts. FIG. 37B. The detected mean percent methylation in each sample with Detected our Low Level qualitative results is displayed at each incubation time point.

FIG. 38 shows a table with methylated and nonmethylated copy numbers detected in each assay well for all 11 slide extracts at each of the three time points. Resulting qualitative calls and associated at-value percent methylation are included.

FIG. 39A-39C show examples of quality-compromised raw data in select wells analyzed during testing of post-conversion refrigerated storage time. 39A. Day 0 QC1 failure of the 10% control prevented comparison with data obtained for prolonged storage, including Day 7 data. 39B. Low amplitude signal on Day 3 caused distortion of observed percent methylation in the 20% control. 39C. Atypical droplet scatter of unknown origin in the CpG 79-82 reaction well of the 0% control after 14 days of storage.

FIG. 40 shows a table with methylated and nonmethylated copy numbers detected by ddPCR in each assay well for conversion reactions of eight samples stored under refrigeration for the indicated number of days. Resulting qualitative calls and associated at-value percent methylation are included.

FIG. 41 shows a table with methylated and nonmethylated copy numbers detected by ddPCR in each assay well for conversion reactions of eight samples stored frozen at −20° C. for the indicated number of days. Resulting qualitative calls and associated at-value percent methylation are included.

FIG. 42 shows graphs of detected total converted allele copy number after post-conversion storage under refrigeration (top panels) or at −20° C. (bottom panels). Data relative to Day 0 are shown for both reaction wells. Individual data points for each sample are included (gray circles) and overlayed with the mean±standard deviation at each timepoint for the entire sample cohort.

FIG. 43 shows 2D data plots observed for three patient samples included in the EtOH contamination studies. Data from well 1 (top panels) and well 2 (bottom panels) is shown. Net methylated (M) and nonmethylated (NM) allele copy numbers and resulting at-value percent methylation in each reaction well are provided.

FIG. 44 shows a loss of detectable amplified copy numbers (methylated+nonmethylated) and signal amplitude with increasing EtOH content of the sample.

FIG. 45 shows results of SNP search of the assay amplicon region on the UCSC Genome Browser site.

FIG. 46 shows a table with a list of SNPs identified by gnomAD in the forward primer (green), probe (yellow) or reverse primer (orange) complementary regions of the assay amplicon. Bolded line items identify SNPs that occur above 0.10% frequency in any of the database populations.

FIG. 47 shows Qiagen ScreenGel report showing PCR products obtained using ddPCR assay primers and PCR conditions.

FIGS. 48A and 48B show a plate layout for the validated 2-well duplex test design (A) and the multiplexed single-well revised test design (B). Oligonucleotides included in each test well are listed underneath. Refer to Table 1 for primer and probe sequences.

FIGS. 49A-C show raw data plots. FIG. 49A showsraw data plots with gating thresholds of a Not Detected patient sample. FIG. 49B shows raw data plots with gating thresholds of patient sample with Low Level (<25%) methylation. FIG. 49C shows raw data plots with gating thresholds of patient sample with Detected methylation.

FIG. 50 is a table showing results comparison of two-well and single-well multiplex MGMT methylation testing for a limited set of controls and patient samples. Allele copy numbers and calculated percent methylation in each CpG group and as the CpG75-82 average are provided. Coefficients of variability (in %) are calculated for all samples with Low Level or Detected outcomes.

FIG. 51 shows the MGMT promoter region amplicon with oligonucleotide sequences used in the single-well multiplex assay. The bisulfite converted, unmethylated sense strand sequence of the 98 bp amplicon is shown. Converted cytosine locations appear as underlined T nucleotides. Probe sequences with complementarity to the methylated (M) or unmethylated (U) CpG sites are shown and fluorescent labels used for each probe are indicated. Probe sequences in sense orientation are shown above, antisense oriented probes are shown below the amplicon sequence.

FIG. 52 shows an example testing for assay interference by inclusion of multiple probes in the same amplicon sequence. Fully methylated and low methylated control samples were tested in a duplex ddPCR assay that included FAM and HEX linked probes for the CpG75-78 region (top panels). For each control sample a second test well containing FAM and HEX linked probes to both CpG groups (75-78 and 79-82) was included in the same experiment (bottom panels). Signal amplitude was evaluated and all plots shown have identical axis limits. We observed that in the presence of a all four probes, fluorescent signal appears additive when compared to the dual-probe test wells, indicating that in the presence of target sequence hydrolysis of probes within the same amplicon occurs independently. No interference of CpG75-78 and CpG79-82 probes was detected.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a primer” includes a plurality of such primers, reference to “the primer” is a reference to one or more primers and equivalents thereof known to those skilled in the art, and so forth.

The word “or” as used herein means any one member of a particular list and also includes any combination of members of that list.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer may refer to inhibiting survival, growth, and/or spread of cancer cells. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the terms “administering” and “administration” refer to any method of providing a treatment (e.g. alkylating agent) to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject.

As used herein, “sample” is meant to mean a specimen taken from an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The term “percent (%) homology” is used interchangeably herein with the term “percent (%) identity” and refers to the level of nucleic acid or amino acid sequence identity when aligned with a wild type sequence or sequence of interest using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., any of the MTS sequences, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res. 25:3389-3402, 1997.) A preferred alignment of selected sequences in order to determine “% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

Substitutions, deletions, insertions or any combination thereof may be used to arrive at a final derivative, variant, or analog. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3, 4 nucleotide base changes as compared to the parent or reference sequence (e.g., primers or probes herein) of the invention, and shares or improves biological function, specificity and/or activity of the parent sequence. Thus, a “variant sequence” can be one with the specified identity to the disclosed probes, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. a MGMT probe or primer).

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Nucleic Acid Sequences

Disclosed are methylation-independent, conversion-dependent primers. In some aspects, the methylation-independent, conversion-dependent primers are GGATATGTTGGGATAGTT (SEQ ID NO:1) or CCCAAACACTCACCAAAT (SEQ ID NO:2). In some aspects, the primers can be at least 75%, 80%, 85%, 90%, or 99% identical to SEQ ID NO:1 or SEQ ID NO:2.

Disclosed are probes. In some aspects, the probes can span two or more CpGs. In some aspects, the probes can be specific for non-methylated sequences or specific for methylated sequences. In some aspects, the probes can be AAACCTACAAACATCAAAACACAAAAC (SEQ ID NO:3), AACCTACGAACGTCGAAACGCAAAAC (SEQ ID NO:4), TAGGTTTTTGTGGTGTGTATTGTTTG (SEQ ID NO:5), TAGGTTTTCGCGGTGCGTATCGTTTG (SEQ ID NO: 6), TTTAGAACGTTTTGCGTTTTCGACGTTCGTAGGT (SEQ ID NO:7), or TTTAGAATGTTTTGTGTTTTGATGTTTGTAGGTT (SEQ ID NO:8). In some aspects, the probes can be at least 75%, 80%, 85%, 90%, or 99% identical to SEQ ID NO:3, 4, 5, 6, 7, or 8.

In some aspects, the disclosed nucleic acid sequences can comprise a detectable moiety (e.g. fluorescent moiety). For example, in some aspects the disclosed nucleic acid sequences can comprise a hexachlorofluorescein (HEX), fluorescein amidites (FAM), Cy5, or Cy5.5 on the 5′ or 3′ end. In some aspects, the disclosed nucleic acid sequences can comprise a quencher, such as ZEN or TAO. In some aspects, other terminal fluorescent labels that can be used include, but are not limited to, ATTO590, ROX (QX600 system) Texas Red, ATT0425, and TAMRA.

Disclosed are nucleic acid sequences between 10 and 50 nucleotides in length. In some aspects, the nucleic acid sequences can be between 15 and 40 nucleotides in length.

### C. Compositions

Disclosed are compositions comprising one or more of the disclosed nucleic acids. In some aspects, the compositions can comprise a mixture of nucleic acid primer and probes. In some aspects, the disclosed compositions can comprise one or more of SEQ ID NOs:1-8.

In some aspects, disclosed are compositions comprising the methylation-independent, conversion-dependent primers: GGATATGTTGGGATAGTT (SEQ ID NO:1) and CCCAAACACTCACCAAAT (SEQ ID NO:2). In some aspects, the compositions comprising nucleic acid sequences at least 75%, 80%, 85%, 90%, 99, or 100% identical to SEQ ID NO:1 and at least 75%, 80%, 85%, 90%, 99, or 100% identical to SEQ ID NO:2.

In some aspects, disclosed are compositions comprising at least two of SEQ ID NOs:3, 4, 5, 6, 7, and 8. In some aspects, the compositions comprise nucleic acid sequences at least 75%, 80%, 85%, 90%, 99, or 100% identical to at least one of SEQ ID NO:3-8 and at least 75%, 80%, 85%, 90%, 99, or 100% identical to at least one of SEQ ID NO:3-8, wherein the two nucleic acid sequences are different.

In some aspects, disclosed are compositions comprising nucleic acid sequences at least 75%, 80%, 85%, 90%, 99, or 100% identical to SEQ ID NO:1 and at least 75%, 80%, 85%, 90%, 99, or 100% identical to SEQ ID NO:2 and further comprising at least two nucleic acid sequences at least 75%, 80%, 85%, 90%, 99, or 100% identical to at least one of SEQ ID NO:3-8 and at least 75%, 80%, 85%, 90%, 99, or 100% identical to at least one of SEQ ID NO:3-8, wherein the two nucleic acid sequences are different.

In some aspects, the disclosed compositions can further comprise a buffer, such as, but not limited to saline, PBS, or water.

Disclosed are compositions comprising two or more of the primers/probes described herein. For example, disclosed are compositions comprising one or more of the oligonucleotides provided in Tables 1 or 2, such as, MGMTddPCR_F, MGMTddPCR_R, HEX 75U-78U, FAM 75M-78M, Cy5.5 79U-82U, and Cy5 79M-82M. Also disclosed are compositions comprising at least MGMTddPCR_F, MGMTddPCR_R, HEX 75U-78U, and FAM 75M-78M. Also disclosed are compositions comprising at least MGMTddPCR_F, MGMTddPCR_R, HEX 79U-82U, and FAM 79U-82U.

### D. Methods

MGMT expression helps with DNA repair. However, if methylated, MGMT is not expressed and DNA damage caused by alkylating agents is not repaired, leading to cell death. Although methylation can be seen as a negative, in some aspects, the presence of sufficient levels of MGMT methylation is associated with improved overall survival after treatment with an alkylating agent, such as TMZ. For example, non-methylated MGMT leads to expression of a DNA damage repair protein that reverts mutagenic O⁶-methylguanine lesions to guanine, thereby preventing cell death and tumorigenesis caused by alkylating agents. In this scenario, the use of an anti-cancer agent that is an alkylating agent would be less effective because the non-methylated MGMT could result in DNA repair therefore saving the cancer cell. Thus, in some aspects, identifying the presence of quantitative data of methylated vs nonmethylated MGMT can help in determining the prognosis or beneficial effects of a treatment in a subject.

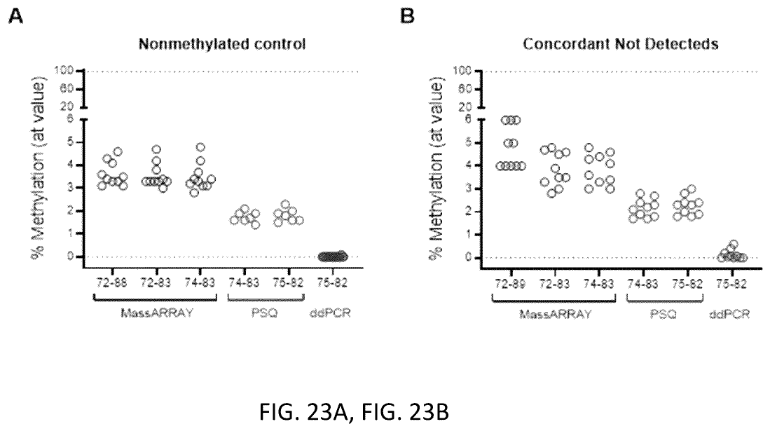

In some aspects, the importance of determining methylation data allows for predicting survival probability and for determining treatment. Many methylation detection protocols have higher assay noise and therefore, cannot detect extremely low methylation levels that can provide valuable information. In some aspects, the use of ddPCR allows for detection of very low methylation levels that are “hidden” in the noise of other methods such as pyrosequencing. For example, FIG. 23 illustrates that the exceptionally low assay noise of ddPCR produces readings for non-methylated controls that are indeed 0% methylated. Pyrosequencing, by contrast, has an assay noise around 2%, so any sample with a methylation status <2% would not be detected by pyrosequencing. This increased assay sensitivity of ddPCR allows for detection of methylation in samples that were previously categorized as Not Detected for MGMT methylation.

In some aspects, determining methylation status can refer to determining quantitative methylation data. In some aspects, the presence or absence of methylation is irrelevant but the quantity of methylation can be beneficial. In some aspects, quantitative methylation data describes an at-value detected percent methylation over the test sequence (i.e. number of methylated CpGs divided by total number of methylated and nonmethylated CpGs multiplied by 100%). In some aspects, a qualitative methylation status, expressed as “Not Detected”, “Low Level” or “Detected”, can subsequently be assigned using thresholds of quantitative data.

In some aspects, quantitative methylation data comprises detecting the average percent methylated CpGs under a probe region (e.g. 4 CpG sites in each test well, 8 sites total) in a sample. In some aspects, how many or which exact CpGs are methylated in the sample cannot be detected. In some aspects, quantitative methylation data comprises interrogating a sample comprising a mixture of cells (tumor and non-tumor origin) that contain between 0 and 8 methylated CpGs in the assay target region. In some aspects, the average of the collective cells in the sample provides the quantitative methylation data.

In some aspects, determining methylation status comprises using two probe sets to interrogate four CpGs in two different wells (so a total of eight CpGs, four in each well). Thus, in some aspects, the disclosed methods can be considered two well assays, duplex assays, or multiwell assays. In some aspects, determining methylation status comprises using three probe sets to interrogate eight CpGs in a single well. In some aspects, a multiplex assay (e.g. single well assay) only has two probe sets (with each probe set comprised of two probes), for a total of four probes and two primers to interrogate eight CpG sites. In some aspects, the disclosed methods can be considered a single-well assay or a multi-well assay. In some aspects, the difference in a single well assay and multi-well assay are the number of detectable moieties (e.g., fluorophores) used to detect methylated CpGs in each well. In some aspects, a multi-well assay can comprise one to two detectable moieties per well. In some aspects, a single-well assay comprises up to six or eight detectable moieties in a single well.

### 1. Determining Methylation Status

Disclosed are methods of determining methylation status of a target nucleic acid sequence in a sample comprising determining quantitative methylation data at two or more predetermined methylation sites in the target nucleic acid sequence, wherein the target nucleic acid sequence comprises an O6-methylguanine-DNA-methyltransferase (MGMT) promoter.

In some aspects, determining the quantitative methylation data comprises an amplification based assay. Thus, disclosed are methods of determining quantitative methylation data of a target nucleic acid sequence in a sample comprising amplifying a target nucleic acid sequence in the sample, wherein the target nucleic acid sequence comprises a MGMT promoter; and determining quantitative methylation data at two or more predetermined methylation sites in the target nucleic acid sequence. In some aspects, the amplification based assay or amplifying step is droplet digital PCR (ddPCR). In some aspects, ddPCR is a technique that allows amplification of a single DNA template from a minimally diluted sample, thus, generating amplicons that are exclusively derived from one template and can be detected with different fluorophores or sequencing. For a review of the digital PCR methodology, see, e.g., Pohl et al., Expert Rev. Mol. Diagn., 4(1):41-7 (2004). In some aspects, instead of performing one reaction per well, ddPCR involves partitioning the PCR solution into tens of thousands of nano-liter sized droplets, where a separate PCR reaction takes place in each droplet. In some aspects, ddPCR has advantages such as reliable workflow, existing instrumentation, moderate cost, highly quantitative, and low noise.

In some aspects, ddPCR comprises methylation-independent conversion-dependent primers and at least two probes that span two or more CpGs of the MGMT promoter. In some aspects, methylation-independent conversion-dependent primers can bind regardless of methylation status but are dependent on converted DNA. For example, in some aspects, a bisulfite conversion protocol can be performed prior to adding the methylation-independent conversion-dependent primers in order to allow the change of unmethylated cytosines into uracils (but leaving the methylated cytosines unchanged). In some aspects, an alternative to conversion by the bisulfite method, can be conversion by a series of enzymatic reactions. In some aspects, the methylation-independent, conversion-dependent primers are GGATATGTTGGGATAGTT (SEQ ID NO:1) or CCCAAACACTCACCAAAT (SEQ ID NO:2). In some aspects, the methylation-independent, conversion-dependent primers can be at least 75%, 80%, 85%, 90%, 99, or 100% identical to SEQ ID NO:1 and at least 75%, 80%, 85%, 90%, 99, or 100% identical to SEQ ID NO:2.

In some aspects, of the at least two probes that span two or more CpGs, one is specific for non-methylated sequences and one is specific for methylated sequences. In some aspects, the at least two probes are specific to converted DNA (i.e. bind to DNA after bisulfite conversion). In some aspects, the at least two probes are AAACCTACAAACATCAAAACACAAAAC (SEQ ID NO:3), AACCTACGAACGTCGAAACGCAAAAC (SEQ ID NO:4), TAGGTTTTTGTGGTGTGTATTGTTTG (SEQ ID NO:5), TAGGTTTTCGCGGTGCGTATCGTTTG (SEQ ID NO:6), TTTAGAACGTTTTGCGTTTCGACGTTCGTAGGT (SEQ ID NO:7), or TTTAGAATGTTTTGTGTTTTGATGTTTGTAGGTT (SEQ ID NO:8). In some aspects, the probes comprise at least two nucleic acid sequences at least 75%, 80%, 85%, 90%, 99, or 100% identical to at least one of SEQ ID NO:3-8 and at least 75%, 80%, 85%, 90%, 99, or 100% identical to at least one of SEQ ID NO:3-8, wherein the two nucleic acid sequences are different.

In some aspect, the two or more predetermined methylation sites are two or more of CpGs 72-83 of the MGMT promoter. In some aspect, the two or more predetermined methylation sites are two or more of CpGs 74-83 of the MGMT promoter. In some aspect, the two or more predetermined methylation sites are two or more of CpGs 75-82 of the MGMT promoter.

In some aspects, the sample is from a subject having glioma. In some aspects, the sample is from tumor tissue. In some aspects, the sample is from a liquid or solid biopsy sample. In some aspects, the sample can be any biological sample from which DNA can be extracted.

In some aspects, the disclosed methods can further comprise, before determining quantitative methylation data, a step of obtaining a sample comprising the target nucleic acid sequence. For example, the sample can be obtained from a subject having glioma.

In some aspects, the disclosed methods further comprise a step of treating when the quantitative methylation data is equal to or above a threshold. In some aspects, the threshold for the MGMT quantitative methylation data can vary depending on the method of methylation detection. In some aspects, the threshold can be 5-10% methylation. Thus, in some aspects, quantitative methylation data showing at least 5% or at least 10% methylation is indicative that the subject should be treated with a standard of care cancer treatment, such as an alkylating agent. In some aspects, treating comprises administering a cancer therapeutic. In some aspects, the cancer therapeutic can be an alkylating agent. In some aspects, the alkylating agent can be, but is not limited to, Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin.

### 2. Treating

Disclosed are methods of diagnosing a subject having cancer as susceptible to treatment with an alkylating agent comprising determining, from a sample, quantitative methylation data at two or more predetermined methylation sites in a MGMT promoter, wherein the sample is from the subject having cancer; and diagnosing the subject as susceptible to treatment with an alkylating agent when the quantitative methylation data at two or more predetermined methylation sites is at or above a threshold. In some aspects, the methods further comprise treating the subject with an alkylating agent when the quantitative methylation data at two or more predetermined methylation sites is at or above a threshold.

Disclosed are methods of treating a subject having cancer comprising determining, from a sample, quantitative methylation data at two or more predetermined methylation sites in a MGMT promoter, wherein the sample is from the subject having cancer; and treating the subject for cancer when the quantitative methylation data at two or more predetermined methylation sites is at or above a threshold.

In some aspects, the threshold for the MGMT quantitative methylation data that separates a poor prognosis from a better prognosis can vary depending on the method of methylation detection. In some aspects, the threshold that separates a poor prognosis from a better prognosis is 5-10% methylation. Thus, in some aspects, quantitative methylation data showing at least 5% or at least 10% methylation is indicative of a better prognosis for a subject to respond to standard of care treatment, such as temozolomide (TMZ) treatment. In some aspects, the threshold is at least 5% methylation.

In some aspects, whether or not to treat a subject with a particular therapeutic can depend on the quantitative methylation data obtained from a sample from the subject. In some aspects, if the MGMT quantitative methylation data is at or above a threshold then a subject can have a better prognosis with a cancer treatment. In some aspects, if the MGMT quantitative methylation data is below a threshold then a subject can have a poorer prognosis with a cancer treatment.

In some aspects, determining the quantitative methylation data comprises an amplification based assay. Thus, disclosed are methods of treating a subject having cancer comprising amplifying a target nucleic acid sequence from a sample from the subject, wherein the target nucleic acid sequence comprises a MGMT promoter; determining quantitative methylation data at two or more predetermined methylation sites in the target nucleic acid sequence; and treating the subject for cancer when the quantitative methylation data at two or more predetermined methylation sites is at or above a threshold. In some aspects, the amplification based assay or amplifying step is droplet digital PCR (ddPCR).

In some aspects, ddPCR comprises methylation-independent conversion-dependent primers and at least two probes that span two or more CpGs of the MGMT promoter. In some aspects, methylation-independent conversion-dependent primers can bind regardless of methylation status but are dependent on converted DNA. For example, in some aspects, a bisulfite conversion protocol can be performed prior to adding the methylation-independent conversion-dependent primers in order to allow the change of unmethylated cytosines into uracils (but leaving the methylated cytosines unchanged). In some aspects, an alternative to conversion by the bisulfite method, can be conversion by a series of enzymatic reactions. In some aspects, the methylation-independent, conversion-dependent primers are GGATATGTTGGGATAGTT (SEQ ID NO:1) or CCCAAACACTCACCAAAT (SEQ ID NO:2). In some aspects, the methylation-independent, conversion-dependent primers can be at least 75%, 80%, 85%, 90%, 99, or 100% identical to SEQ ID NO:1 and at least 75%, 80%, 85%, 90%, 99, or 100% identical to SEQ ID NO:2.

In some aspects, of the at least two probes that span two or more CpGs, one is specific for non-methylated sequences and one is specific for methylated sequences. In some aspects, the at least two probes are specific to converted DNA (i.e. bind to DNA after bisulfite conversion). In some aspects, the at least two probes are AAACCTACAAACATCAAAACACAAAAC (SEQ ID NO:3), AACCTACGAACGTCGAAACGCAAAAC (SEQ ID NO:4), TAGGTTTTTGTGGTGTGTATTGTTTG (SEQ ID NO:5), TAGGTTTTCGCGGTGCGTATCGTTTG (SEQ ID NO:6), TTTAGAACGTTTTGCGTTTCGACGTTCGTAGGT (SEQ ID NO:7), or TTTAGAATGTTTTGTGTTTTGATGTTTGTAGGTT (SEQ ID NO:8). ). In some aspects, the probes comprise at least two nucleic acid sequences at least 75%, 80%, 85%, 90%, 99, or 100% identical to at least one of SEQ ID NO:3-8 and at least 75%, 80%, 85%, 90%, 99, or 100% identical to at least one of SEQ ID NO:3-8, wherein the two nucleic acid sequences are different.

In some aspect, the two or more predetermined methylation sites are two or more of CpGs 72-83 of the MGMT promoter. In some aspect, the two or more predetermined methylation sites are two or more of CpGs 74-83 of the MGMT promoter. In some aspect, the two or more predetermined methylation sites are two or more of CpGs 75-82 of the MGMT promoter.

In some aspects, the sample is from a subject having glioma. In some aspects, the sample is from tumor tissue. In some aspects, the sample is from a liquid or solid biopsy sample. In some aspects, the sample can be any biological sample from which DNA can be extracted.

In some aspects, the disclosed methods can further comprise, before determining quantitative methylation data, a step of obtaining a sample comprising the target nucleic acid sequence. For example, the sample can be obtained from a subject having glioma.

In some aspects, treating comprises radiotherapy and/or chemotherapy. In some aspects, treating comprises administering a cancer therapeutic. In some aspects, the cancer therapeutic can be an alkylating agent. In some aspects, the alkylating agent can be, but is not limited to, Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin.

### 3. Assessing Survival Probability

Disclosed are methods of assessing survival probability in a subject having cancer comprising determining, from a sample, quantitative methylation data at two or more predetermined methylation sites in a target nucleic acid sequence, wherein the sample is from the subject having cancer, wherein the target nucleic acid sequence comprises a MGMT promoter, wherein quantitative methylation data below a threshold indicates decreased survival probability. In some aspects, the survival probability is based on the quantitative methylation data and the subject's treatment with an alkylating agent. For example, in some aspects, quantitative methylation data equal to or above a threshold indicates an increased survival probability in those subjects treated with an alkylating agent. In some aspects, assessing survival probability in a subject having cancer can be used interchangeably with predicting survival of a subject having cancer.

In some aspects, the percent methylation that is quantitatively detected can be both a function of how many tumor cells are contained in the sample (tumor purity) and how much methylation is present in the tumor cells. For example, a 20% methylated sample and a 50% methylated sample can simply contain fewer or more tumor cells with an equal level of methylation. Thus, in some aspects, the detection or lack of detection based on the quantitative methylation threshold can be used to assess survival probability and inform the treatment process.

In some aspects, determining the quantitative methylation data comprises an amplification based assay. Thus, disclosed are methods of determining quantitative methylation data of a target nucleic acid sequence in a sample comprising amplifying a target nucleic acid sequence in the sample, wherein the target nucleic acid sequence comprises a MGMT promoter; and determining quantitative methylation data at two or more predetermined methylation sites in the target nucleic acid sequence. In some aspects, the amplification based assay or amplifying step is droplet digital PCR (ddPCR).

In some aspects, ddPCR comprises methylation-independent conversion-dependent primers and at least two probes that span two or more CpGs of the MGMT promoter. In some aspects, methylation-independent conversion-dependent primers can bind regardless of methylation status but are dependent on converted DNA. For example, in some aspects, a bisulfite conversion protocol can be performed prior to adding the methylation-independent conversion-dependent primers in order to allow the change of unmethylated cytosines into uracils (but leaving the methylated cytosines unchanged). In some aspects, an alternative to conversion by the bisulfite method, can be conversion by a series of enzymatic reactions. In some aspects, the methylation-independent, conversion-dependent primers are GGATATGTTGGGATAGTT (SEQ ID NO:1) or CCCAAACACTCACCAAAT (SEQ ID NO:2). In some aspects, the methylation-independent, conversion-dependent primers can be at least 75%, 80%, 85%, 90%, 99, or 100% identical to SEQ ID NO:1 and at least 75%, 80%, 85%, 90%, 99, or 100% identical to SEQ ID NO:2.

In some aspects, of the at least two probes that span two or more CpGs, one is specific for non-methylated sequences and one is specific for methylated sequences. In some aspects, the at least two probes are specific to converted DNA (i.e. bind to DNA after bisulfite conversion). In some aspects, the at least two probes are AAACCTACAAACATCAAAACACAAAAC (SEQ ID NO:3), AACCTACGAACGTCGAAACGCAAAAC (SEQ ID NO:4), TAGGTTTTTGTGGTGTGTATTGTTTG (SEQ ID NO:5), TAGGTTTTCGCGGTGCGTATCGTTTG (SEQ ID NO:6), TTTAGAACGTTTTGCGTTTCGACGTTCGTAGGT (SEQ ID NO:7), or TTTAGAATGTTTTGTGTTTTGATGTTTGTAGGTT (SEQ ID NO:8). In some aspects, the probes comprise at least two nucleic acid sequences at least 75%, 80%, 85%, 90%, 99, or 100% identical to at least one of SEQ ID NO:3-8 and at least 75%, 80%, 85%, 90%, 99, or 100% identical to at least one of SEQ ID NO:3-8, wherein the two nucleic acid sequences are different.

In some aspect, the two or more predetermined methylation sites are two or more of CpGs 72-83 of the MGMT promoter. In some aspect, the two or more predetermined methylation sites are two or more of CpGs 74-83 of the MGMT promoter. In some aspect, the two or more predetermined methylation sites are two or more of CpGs 75-82 of the MGMT promoter.

In some aspects, the sample is from a subject having glioma. In some aspects, the sample is from tumor tissue. In some aspects, the sample is from a liquid or solid biopsy sample. In some aspects, the sample can be any biological sample from which DNA can be extracted.

In some aspects, the disclosed methods can further comprise, before determining quantitative methylation data, a step of obtaining a sample comprising the target nucleic acid sequence. For example, the sample can be obtained from a subject having glioma.

In some aspects, the disclosed methods further comprise a step of treating (administering) when the quantitative methylation data is equal to or above a threshold. In some aspects, the threshold for the MGMT quantitative methylation data can vary depending on the method of methylation detection. In some aspects, the threshold can be 5-10% methylation. Thus, in some aspects, quantitative methylation data showing at least 5% or at least 10% methylation is indicative that the subject should be treated with a standard of care cancer treatment, such as an alkylating agent. In some aspects, treating comprises administering a cancer therapeutic. In some aspects, the cancer therapeutic can be an alkylating agent. In some aspects, the alkylating agent can be, but is not limited to, Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin.

### E. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for determining quantitative methylation data, the kit comprising at least two probes that span two or more CpGs of the MGMT promoter and methylation-independent conversion-dependent primers. In some aspects, the kit comprises any of the primers or probes described herein. For example, the disclosed kits can comprise one or more nucleic acid sequences of SEQ ID NOs:1-8.

### EXAMPLES

### A. Example 1—Duplex or Multi-Well Assay

### 1. Background

DNA methylation is a principal mechanism for epigenetic regulation of mammalian genomes. Methylation occurs on the fifth carbon of cytosines, giving rise to 5-methylcytosine (5 mC). In humans, 5 mC is predominantly observed in the context of symmetrical CpG dinucleotides. The haploid human methylome contains nearly 29 million CpG sites, rendering the bulk genome depleted for this dinucleotide sequence. The exception are CpG islands (CGIs), short (˜0.2-2 kb) genomic regions that are enriched for CpGs and are frequently associated with promoters of housekeeping and developmentally regulated genes. While 70-80% of the human methylome is in its methylated state, CpG sites in CGIs, promoters and first exons are notably nonmethylated in somatic tissue. Cytosine methylation is generally linked to transcriptional regulation, although the underlying mechanism appear to be distinct within the genomic context of the methylated region. At CpG-rich promoters and their associated CGIs transcriptional activity is negatively correlated with hypermethylation.

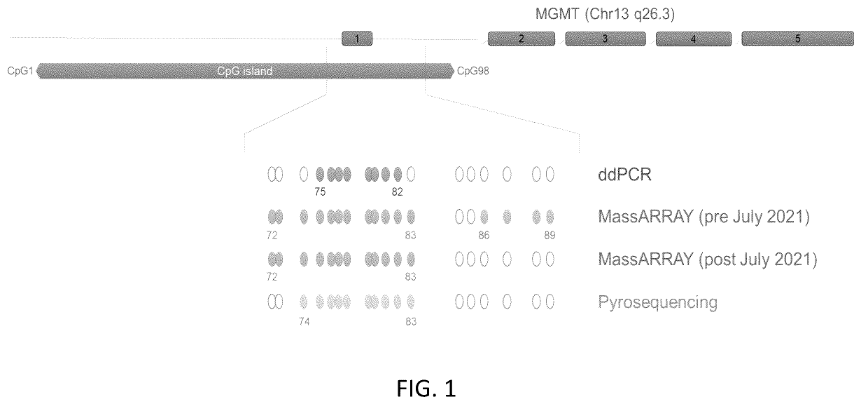

For the O⁶-methylguanine-DNA methyltransferase (MGMT) gene, methylation of its promoter-associated CGI is the predominant mode of expression regulation. The 762 bp CpG island, which contains a total of 98 CpG sites, is located at the 5′ end of the gene and encompasses the upstream regulatory region, exon 1 and part of intron 1 (FIG. 1). The MGMT tumor suppressor gene encodes a DNA damage repair protein that reverts mutagenic O⁶-methylguanine lesions to guanine, thereby preventing cell death and tumorigenesis caused by alkylating agents. In cancer therapy, alkylating agents are common first-line chemotherapy agents and expression of MGMT in the tumor tissue can modulate the response to treatment. The relationship between MGMT promoter methylation, MGMT expression and resistance to chemotherapy is of particular importance in the context of glioblastoma (GBM).

Glioblastoma is the most common malignant tumor of central nervous system (CNS), accounting for 54% of all adult malignant CNS tumors and an estimated 12000 newly diagnosed cases in the U.S. in 2021. Glioblastoma is more common in males than females, with incidence rates of 4.03 and 2.54 per 100000, respectively. Diagnosis of primary glioblastoma generally occurs in adults >50 years of age and incidence rates are highest for individuals between 70 and 85. Current standard of care therapy for newly diagnosed GBM includes maximal safe resection and adjuvant radiotherapy (RT) and chemotherapy with the methylating agent temozolomide (TMZ).

Numerous clinical studies provide evidence that TMZ treatment has improved efficacy in GBM patients for which MGMT expression is silenced by MGMT promoter methylation. Overall median survival as determined by meta-analysis was 14.1 months for treatment of unmethylated GBM by RT/TMZ, while median survival in the MGMT methylation-positive patient group was improved to 24.6 months. Accordingly, MGMT promoter methylation status in GBM tissue is considered an important prognostic marker and MGMT methylation testing is included in the recommended diagnostic procedures for GBM as stated in current NCCN guidelines.

A wide range of detection methods have been employed to detect MGMT methylation (Table 1), with Methylation-Specific PCR approaches and Pyrosequencing the most commonly used for clinical testing.

TABLE 1
Overview of select methodologies used for detection of MGMT promoter methylation.

| Method | Method principle |
|---|---|
| Methylation-sensitive restriction and | DNA restriction by methylation-sensitive |
| Southern blotting | endonuclease followed by detection of differential |
| | restriction pattern by Southern blot |
| Methylation-sensitive restriction and | DNA restriction by methylation-sensitive |
| PCR | endonuclease followed by PCR to amplify |
| | methylated amplicons only |
| Bisulfite conversion and sanger | Bisulfite conversion of DNA followed by Sanger |
| sequencing | sequencing of region of interest |
| Methylation-specific polymerase chain | Bisulfite conversion-specific primers with CpG/TpG |
| reaction (MSP), and real-time | differentiating sequences near the 3′ end of the |
| quantitative MSP (RT-MSP) | primer amplify unmethylated or methylated alleles |
| Pyrosequencing (PSQ) | Primer extension by sequentially dispensed |
| | nucleotides using a bisulfite-converted DNA |
| | template; PPᵢ release upon successful incorporation |
| | of a nucleotide is detected by chemiluminescence |
| Methylation-specific multiplex ligation- | Ligation-dependent probes are designed over CpG |
| dependent probe amplification (MS- | in the context of methylation dependent |
| MLPA) | endonuclease sites; ligation and amplification is |
| | performed with and without restriction digest; |
| | reaction products are quantified by fragment |
| | analysis |
| SNuPE ion pair-reverse phase high- | Primer extension of bisulfite converted DNA; one |
| performance liquid chromatography | primer per site to be analyzed; extension by C or T; |
| | products analyzed by HPLC and quantified by |
| | integration of peaks |
| High Resolution Melting (HRM) | PCR amplification of bisulfite converted DNA |
| | followed by high resolution melting curve analysis |
| | to detect differential melting peaks from methylated |
| | and nonmethylated alleles |
| Matrix-Assisted Laser | PCR amplification of bisulfite converted DNA |
| Desorption/Ionization-Time Of Flight | followed by in vitro transcription, enzymatic |
| (MALDI-TOF, Mass Spectrometry) | cleavage and resolution of fragments by mass |
| | spectrometry; fragment size differences occur as a |
| | result of methylation |

MGMT methylation studies and clinical trials have evaluated methylation status of a range of CpG sites within the MGMT promoter, ranging from single site analysis to more comprehensive studies of up to 61 CpGs, with the most commonly studied region encompassing CpGs 74-78. Methylation patterns were generally found to be heterogeneous and no individual CpG site has thus far been found to be singularly important for the regulation of MGMT protein expression. However, several studies confirm that methylation of the region spanning CpGs 73-81 correlates with gene expression and, more importantly for clinical testing, with prognosis in patients with newly diagnosed glioblastoma patients.

Correlation of the quantitative level of MGMT methylation and survival has been explored in multiple clinical studies, the majority of which employed a pyrosequencing or MSP method. The clinically relevant cutoff values in these reports ranged from approximately 8-25%, with a 10% threshold widely used in clinical testing, including by the MassARRAY test currently performed at ARUP.

Purpose of this study is to validate a droplet digital polymerase chain reaction (ddPCR) test for detection of MGMT promoter methylation at CpGs 75-82. A ddPCR approach has been used for detection of methylation in single or few CpG sites in genes other than MGMT and this assay established that this methodology can be applied to the MGMT locus.

The ddPCR testing methodology and instrumentation is used for several other clinical tests (U.S. Pat. Nos. 3,002,956, 3,003,751, 2,012,868, 2,013,921) performed by the Molecular Oncology clinical laboratory. The current MGMT MALDI-TOF test is the only test utilizing the Agena MassARRAY instrumentation. A transition to the ddPCR QX200 testing platform will consolidate workflows and reagent needs, lighten cross-platform training requirements and reduce instrument maintenance costs.

The ddPCR test allows for retention of high-throughput capabilities of the MassARRAY while reducing technologist hands-on time and costs per billable test.

While the CpG coverage of the ddPCR assay (CpG 75-82) is reduced from the coverage of the MassARRAY (CpGs 72-83 as of July 2021; FIG. 1), the queried region of the CGI covers the majority of the 73-81 region shown to have clinical significance.

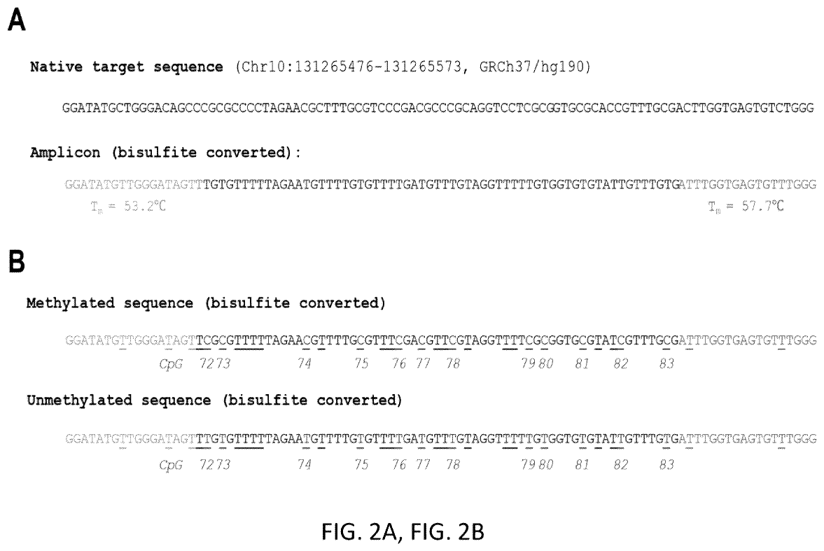

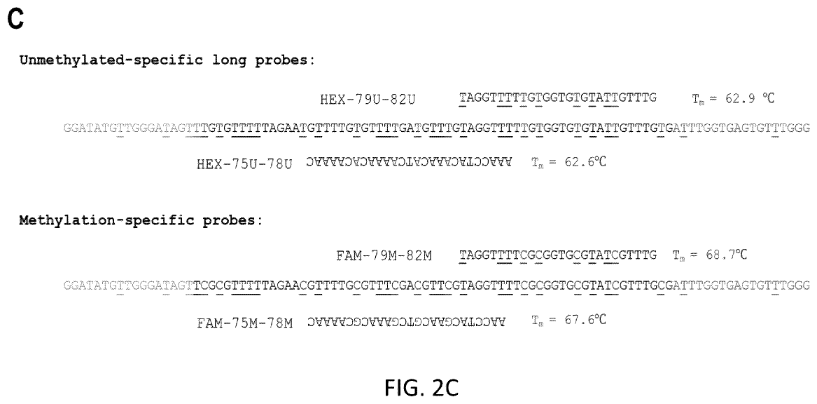

In addition to coverage loss, the granularity of the quantitative resolution, is somewhat reduced for ddPCR when compared to MassARRAY. While MassARRAY was able to approximate the percent methylation over an average of 1-3 CpGs, the ddPCR design (FIG. 2) only allows for determination of mean methylation over two groups of four CpGs. This is not expected to adversely affect the prognostic value of the ddPCR test, as studies have demonstrated that mean percent methylation over four CpGs are sufficient for evaluation of MGMT promoter methylation and for predicting therapeutic response.

Reduced assay noise of the ddPCR method when compared to MassARRAY, potentially allows for methylation detection to greater sensitivity. While the clinical significance of very low MGMT methylation is unknown at this time, future clinical studies may uncover a need for increased sensitivity in MGMT promoter methylation testing.

### 2. Assay Design and Reaction Setup

To detect and quantify MGMT promoter methylation at CpGs 75-82 a two-well ddPCR assay design was validated (Table 2 and FIG. 2). Both test wells utilize a universal methylation-independent, conversion-specific primer pair. The primers are identical to those used for assessing methylation of CpGs 72-83 by the current MALDI-TOF (MassARRAY) test and give rise to a 98-bp amplicon. The duplicate test wells interrogate two distinct sets of four CpGs each (Well 1: 75-78, well 2: 79-82) using differentially labeled probes with complementarity to fully methylated (FAM-label) and fully unmethylated (HEX-label) sequence.

TABLE 2
Primer and probe composition
used for the MGMT methylation ddPCR assay.

| | | | | Final |
|---|---|---|---|---|
| | | | | conc. |
| | 5′ Dye/ | | | in |
| Oligo- | 3′ | Sequence | Tₘ | assay |
| nucleotide | Quencher | 5′-3′ | (° C.) | (μM) |
| MGMTddPCR_F | N/A | GGATATGTTGGGAT | 53.2 | 1 |
| | | AGTT | | |
| | | (SEQ ID NO: 1) | | |
| MGMTddPCR_R | N/A | CCCAAACACTCACC | 57.7 | 1 |
| | | AAAT | | |
| | | (SEQ ID NO: 2) | | |
| HEX 75U-78U | 5′HEX-- | AAACCTACAAACAT | 62.6 | 0.4 |
| | ZEN-- | CAAAACACAAAAC | | |
| | 3′ | (SEQ ID NO: 3) | | |
| | IowaBlack | | | |
| FAM 75M-78M | 5′6FAM-- | AACCTACGAACGTC | 67.6 | 0.4 |
| | ZEN-- | GAAACGCAAAAC | | |
| | 3′ | (SEQ ID NO: 4) | | |
| | IowaBlack | | | |
| HEX 79U-82U | 5′HEX-- | TAGGTTTTTGTGGT | 62.9 | 0.4 |
| | ZEN-- | GTGTATTGTTTG | | |
| | 3′ | (SEQ ID NO: 5) | | |
| | IowaBlack | | | |
| FAM 79M-82M | 5′6FAM-- | TAGGTTTTCGCGGT | 68.7 | 0.4 |
| | ZEN-- | GCGTATCGTTTG | | |
| | 3′ | (SEQ ID NO: 6) | | |
| | IowaBlack | | | |

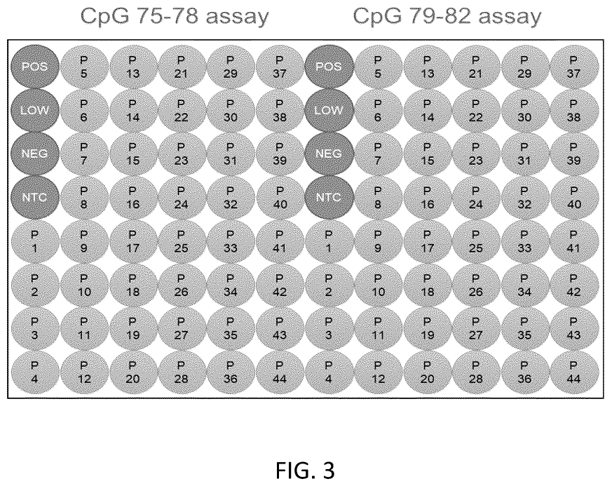

The assay is performed on the Biorad QX200™ droplet digital assay platform, which allows for partitioning of 20 μL PCR reactions into ˜23,000 oil-immersed droplets in a 96-well plate format. Up to 44 patient samples can be evaluated per run, with remaining wells utilized for four in-run controls (Fully methylated, Low methylated, Unmethylated and no template). Each sample is concurrently tested in two reaction wells arranged in a split-plate layout (FIG. 3)

The reaction composition is summarized in Table 3 and thermal cycling conditions are shown in Table 4.

TABLE 3
PCR reaction components in each well.

| | Per 20 μL |
|---|---|
| Reagent | reaction |
| 4X ddPCR Supermix (no dUTP) | 5 μL |
| 20X MGMT primer probe mix | 1 μL |
| MseI restriction enzyme (10 U/μL) | 0.5 μL |
| MBG water | 9.5 μL |
| DNA Template | 4 μL |

TABLE 4
Summary of thermal cycling protocol.

| | | | | Number |
|---|---|---|---|---|
| | Temperature, | | Ramp | of |
| Cycling Step | 0° C. | Time | Rate | Cycles |
| Enzyme activation | 95 | 10 min | 2° C./sec | 1 |
| Denaturation | 94 | 30 sec | | 40 |
| Annealing/extension | 55 | 1 min | | |
| Enzyme | 98 | 10 min | | 1 |
| deactivation | | | | |
| Hold (optional) | 4 | Infinite | 1° C./sec | 1 |

### 3. Data Plots and Interpretation

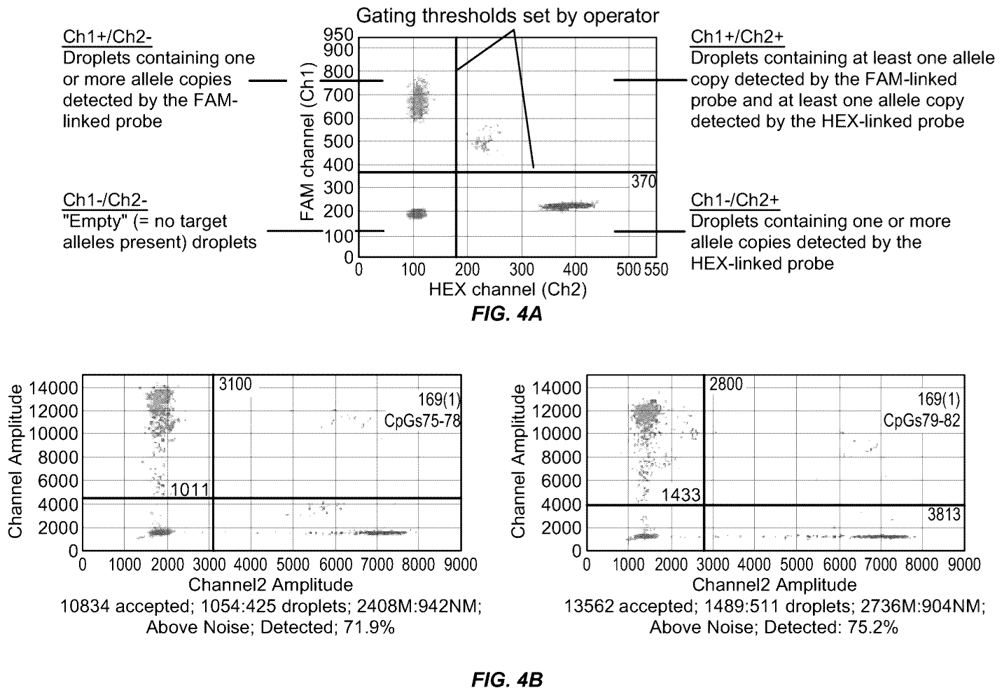

Two data plots are evaluated for each control or patient sample, corresponding to each of the two reaction wells. Vertical and horizontal gates that are set by the operator according to SOP and workbook guidelines divide the data plots into analysis sectors of negative and positive droplet counts in each of the two detection channels (FIG. 4). From the droplet count distribution the detected copy number of methylated and nonmethylated alleles as well as the corresponding percent methylation is calculated by the QXManager Analysis software and the workbook results table.

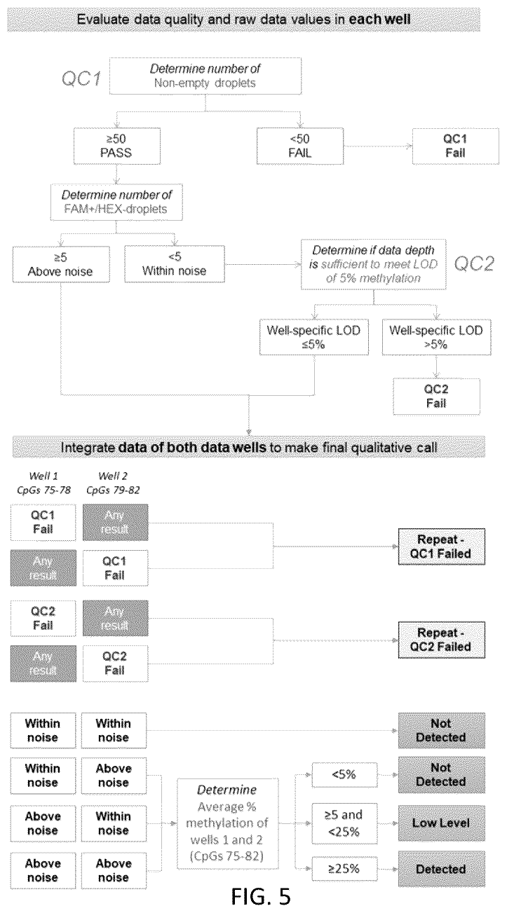

All data interpretation in this validation follows the flowchart in FIG. 5. A minimum of 50 nonempty raw droplet points in each well are required to pass the quality checkpoint (QC) 1, indicating that a minimum of 50 converted genomic target copies must be present to further evaluate the well outcome. Failure at this QC indicates extremely poor data quality and prompts immediate repeat. If the QC1 minimum data requirement is met the droplet counts in the FAM+/HEX− sector are enumerated to determine if the assay results are outside the assay noise established during the development. Wells with <5 droplets in this sector are scored as methylation Not Detected after passing a second quality checkpoint (QC2) that queries the overall data depth in the well sufficiency to meet a 5% sensitivity threshold. Samples with ≥5 FAM+/HEX− droplets are considered above the assay raw data noise. For those cases the resulting percent methylation is calculated and compared to presumed clinically relevant reporting thresholds. Samples with a value percent methylation <5% are considered Not Detected for reporting purposes. Samples with methylation ranging from 5 to <25% are reported as Low Level methylation positive; 25% and above a Detected score is assigned.

Several important nuances are worth noting that are helpful in interpreting ddPCR data in general and the specific outcomes of the MGMT methylation assay by this technology.

As is the case for all digital PCR experiments, the calculated allele copy numbers are the most likely mathematical approximation of the actual copy numbers per well that can be made from the observed ratio of positive and negative reaction partitions (droplets). Generally, the droplet count does not equal the allele copy count because the limiting number of available partitions results at least in partial occupancy of >1 allele copy per droplet and because only part of the 20 μL reaction is successfully partitioned to yield interpretable data (typically 10,000-15,000 droplets out of 23,000 maximal).

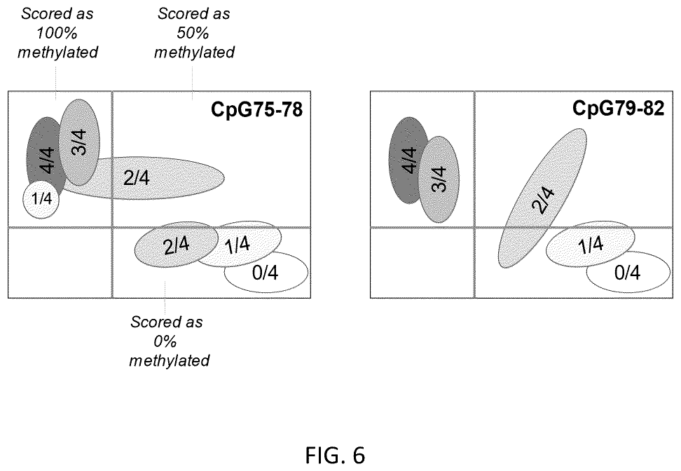

The assay design relies on probes with complementarity to the fully methylated and fully nonmethylated set of four CpGs in each assay well (FIG. 2C). Experiments using synthetic mimics of differentially methylated target alleles (i.e. C or T in each CpG position) during assay development determined the approximate cluster position of the resulting raw data (FIG. 6). The results illustrate that, depending on CpG context, certain combinations of methylation positive and negative CpG sites may result in an overestimation (e.g. alleles with 3 of 4 sites methylated scored as 100% methylated) or underestimation (e.g. alleles with 1 of 4 sites methylated generally scored as 0% methylated). Most importantly, the mapping studies demonstrated that no cross-reactivity of the methylation-specific probe with fully nonmethylated (0/4) targets occurred, as was the case vice versa. As a result, fully nonmethylated and methylated controls return nearly perfect 0% and 100% quantitative outcomes.

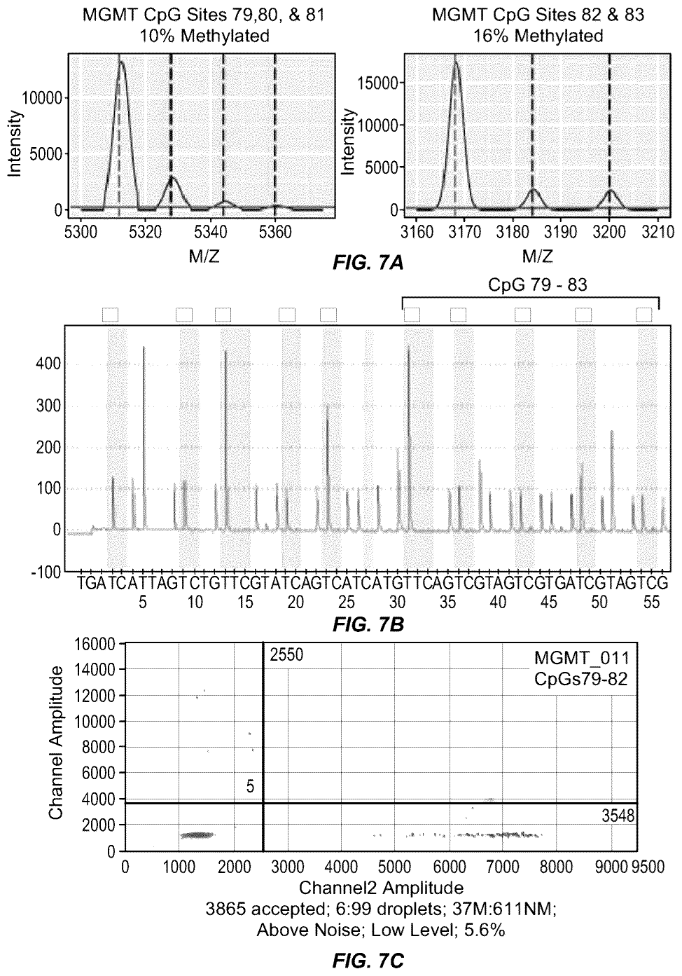

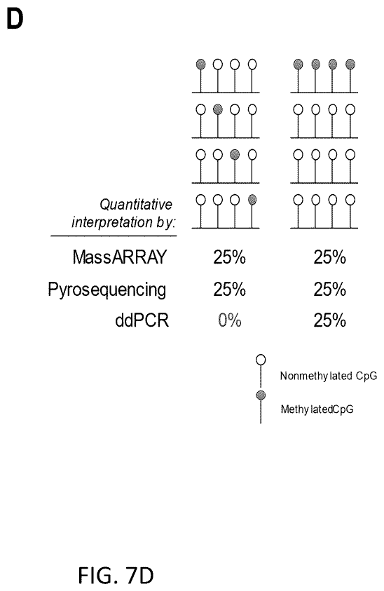

As accuracy studies during development and those included in this validation show, the overall percent methylation generally agrees with other detection methodologies, as underestimated and overestimated individual alleles frequently average out. However, samples enriched in allele counts with highly methylated status (3 of 4 and 4 of 4 sites) may report higher mean percent methylation values compared to methods like pyrosequencing or MALDI-TOF where methylation is quantified by integration of peak heights or of the area under the curve at each individual site or groups of 2-3 sites (FIG. 7A-C). Similarly, underscoring of alleles with single site methylation in low methylation samples may yield lower at value mean percent methylation outcomes compared to the other methodologies. A simple illustration of this phenomenon is provided in FIG. 7D

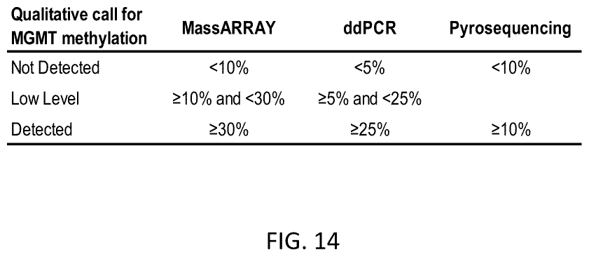

The percent methylation reporting thresholds (5%/25%) for ddPCR data used in this validation were established to maintain consistency of reported outcomes with the current MassARRAY test, with a lowering of reporting thresholds by 5% (see FIG. 14) based on the lower assay noise level in ddPCR (0-0.5% compared to 4-8% by MassARRAY and 2-3% by pyrosequencing, see FIG. 23). While the Accuracy studies shown in this validation show good overall agreement with the resulting qualitative calls between MassARRAY, pyrosequencing and ddPCR, additional studies for clinical validation of the chosen threshold are recommended.

### 4. Validation of Assay Specificity

Assay specificity (by exclusion) was validated by testing 59 FFPE samples with presumed nonmethylated MGMT status. All samples of the specificity cohort were sourced from patient samples submitted for clinical testing by the Microsatellite Instability (MSI) HNPCC/Lynch Syndrome by PCR method. Only tissue extracts from areas identified as the MSI normal control region were utilized. Macrodissected areas ranged from small (10 samples), medium (25 samples) and large (24 samples).

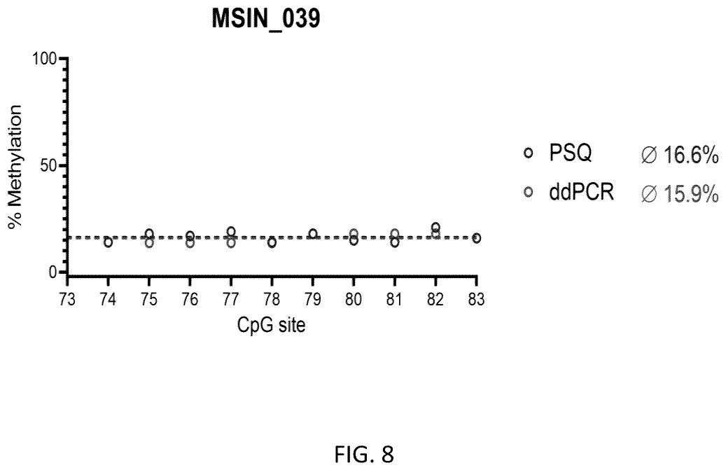

Results are summarized in FIGS. 8-12.

Six samples failed QC requirements in initial and repeated testing (MSIN_003, MSIN_016, MSIN_021, MSIN_23, MSIN_050, MSIN_058). One additional sample (MSIN_008) failed QC in initial testing, with insufficient material remaining for repeat ddPCR analysis. These seven samples were excluded from the specificity cohort.

Of the remaining 52 samples, 42 passed QC checkpoints and returned raw droplet counts within the assay noise (<5 FAM+/HEX−) for both test wells, resulting in a Not Detected qualitative call.

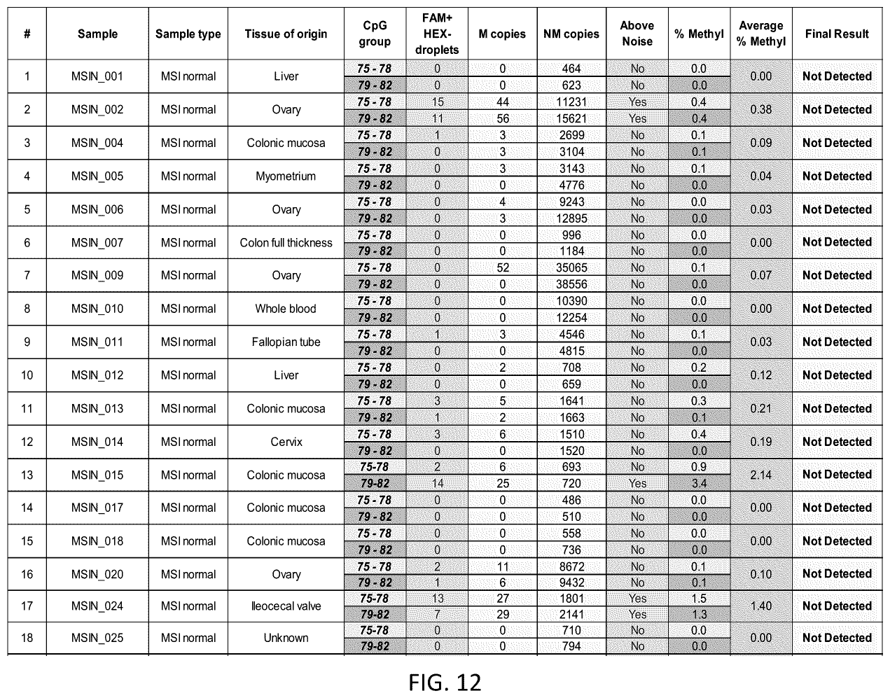

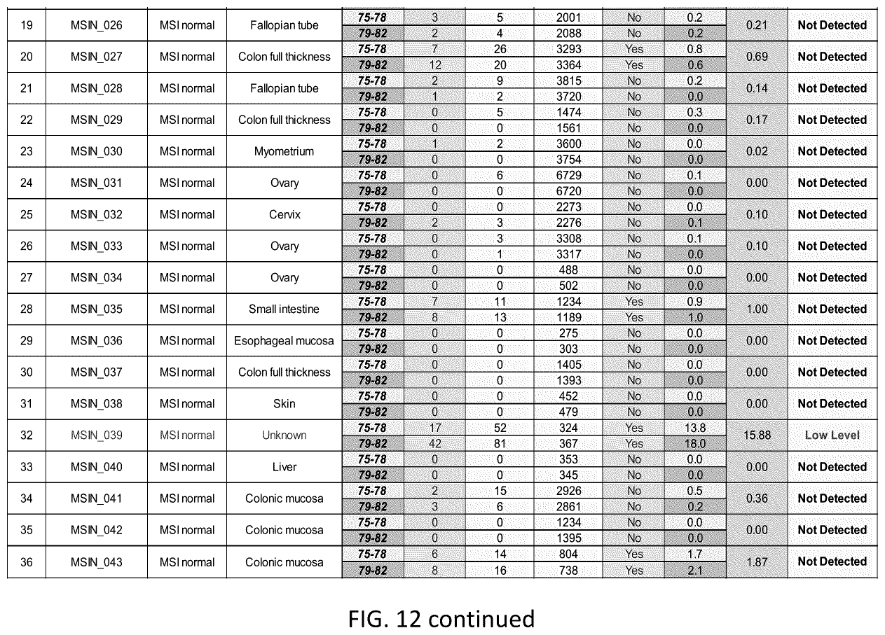

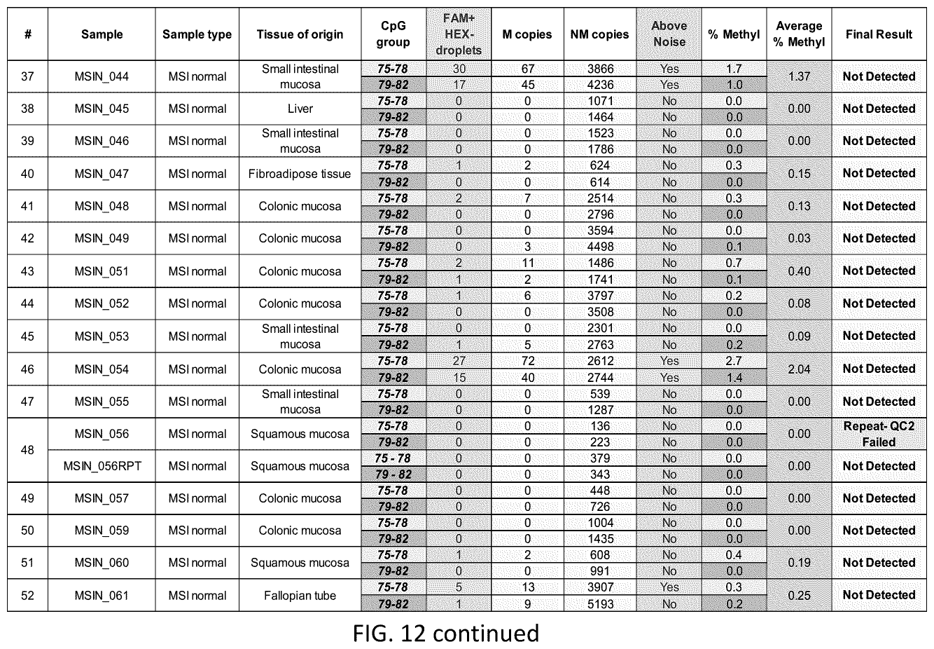

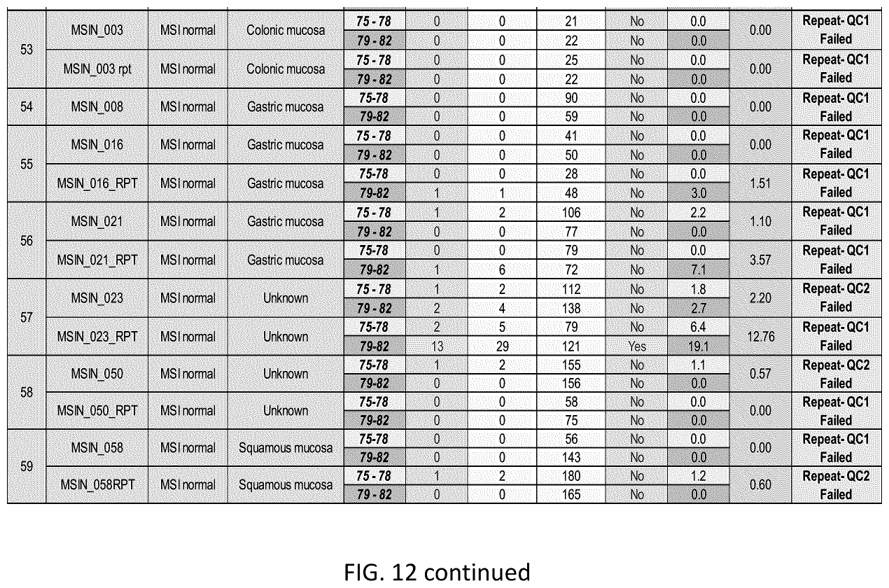

Two samples (MSIN_015 and MSIN_061) showed above noise FAM+/HEX− droplet counts for one test well only, while eight samples were above assay noise for both test wells. Of those, one sample (MSIN_039, FIG. 12) resulted in methylation levels of 15.9%. Subsequent methylation analysis by pyrosequencing confirmed MGMT methylation at a mean value of 16.6% for CpGs 74-83 (see FIG. 8). MSIN_039 was therefore eliminated from the specificity cohort as the assumption of “normal” tissue status was found to be untrue.

All remaining samples with above background raw data signal yielded percent mean methylation levels below 5%, resulting in Not Detected qualitative calls based on the assigned clinical significance threshold.

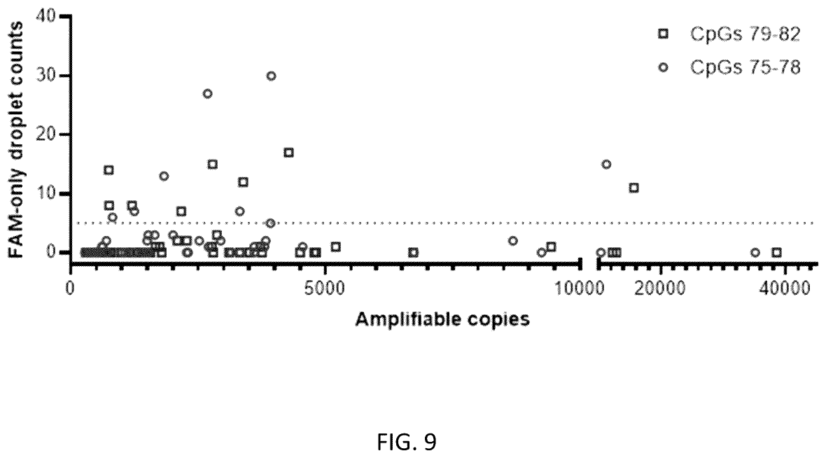

Raw FAM-only droplet counts were independent of total amplifiable copy numbers (i.e. bisulfite converted template DNA input) in the specificity cohort (Pearsons r=0.049 and 0.024 for wells 1 and 2, respectively; FIG. 9). Samples with above noise raw droplet counts were found across nearly the full range of amplifiable copy number (range 746 to 15678 copies per well).

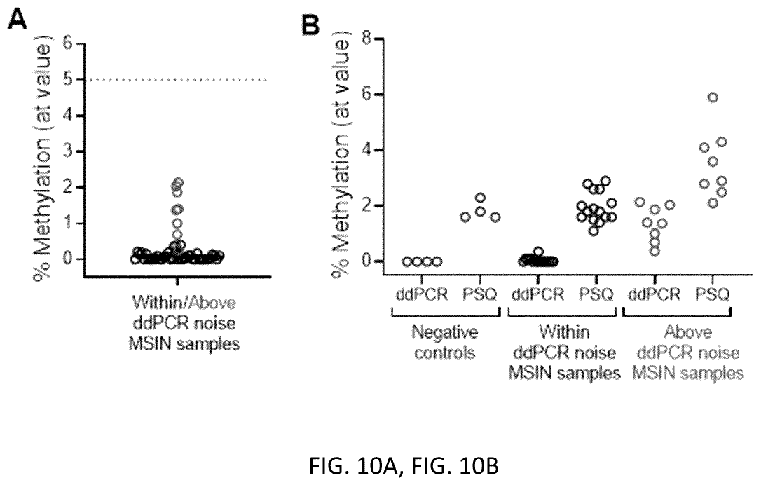

Samples with raw ddPCR droplet counts above noise generally resulted in higher at value percent methylation values (range 0.25-2.14%, mean 1.23%) when compared to samples scoring within the assay noise (range of 0-0.4%, mean 0.07%) although some overlap in percent methylation values was observed between both sub-cohorts (FIG. 10A). When subsets of eight above ddPCR noise samples and 15 within noise samples were also analyzed by pyrosequencing, higher percent methylation values were generally observed for the above noise specimens (range 2.1-5.9%, mean 3.5%) versus within noise samples (range 1.1-2.9%, mean 2.0%), although differences between the groups of samples may be confounded by the higher baseline of percentages measured by pyrosequencing (see Negative controls in FIG. 10B).

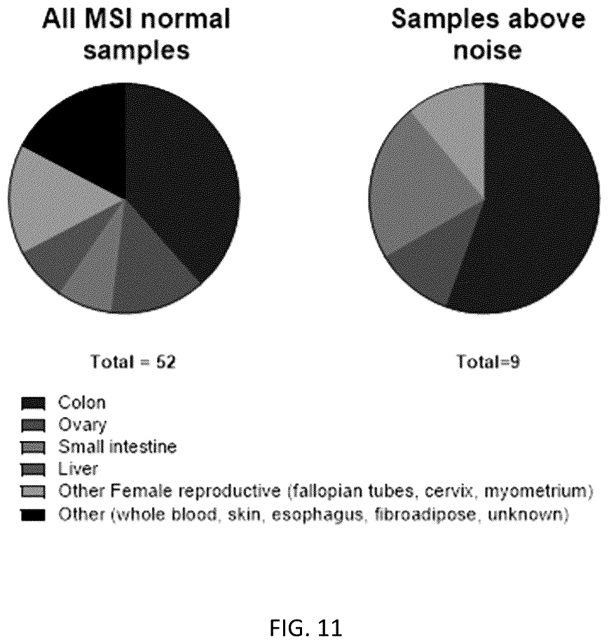

In addition, when tissues of origin were scrutinized for all presumed normal samples compared to above ddPCR noise samples, samples with above assay noise ddPCR signal almost exclusively originated from colonic, ovarian or small intestinal sources (FIG. 11). Abnormal MGMT promoter methylation is associated with colorectal, ovarian and gastrointestinal stromal tumorigenesis. It is therefore plausible that that the presumed normal MSI testing reference samples with above noise raw ddPCR droplet counts represent tissue samples with true but low levels of MGMT methylation.

In summary, of the 51 presumed normal FFPE tissue samples included in the final specificity validation cohort, 42 were scored as qualitatively Not Detected by virtue of yielding raw data within the established ddPCR assay noise. The remaining 9 samples resulted in Not Detected calls based on at value average percent methylation below the clinical significance threshold of 5%.

For the validation cohort, the observed specificity of the assay by exclusion is 100% (51/51). The 95% confidence interval is 95.2-100% (Jeffreys approximation).

### 5. Validation Accuracy

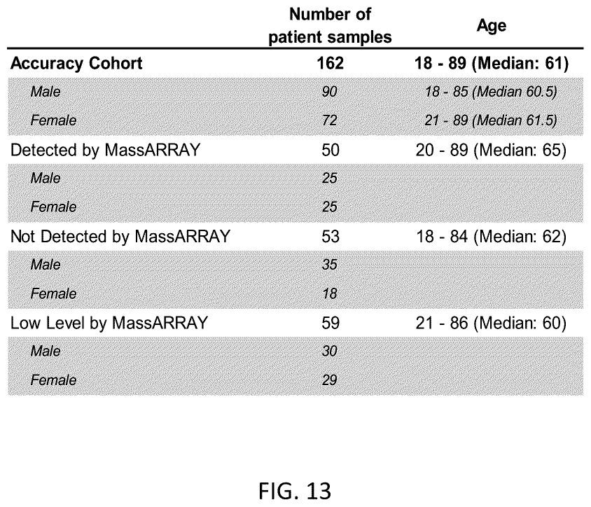

In the context of this validation, “Accuracy” was defined as concordance with qualitative outcomes of the MALDI-TOF MassARRAY assay (2009310) performed by the Molecular Oncology clinical laboratory. A total of 162 patient samples were used for validation of assay accuracy, with original clinical tests performed between April 2020 and May 2021. All DNA extracts generated by Molecular Oncology staff at the time of clinical testing were stored at −20° C. Prior to validation testing, patient samples and associated MassARRAY reports were deidentified under IRB 7275. Basic demographic information of the validation cohort is provided in FIG. 13.

Qualitative ddPCR of the accuracy studies followed the data interpretation key outlined in FIG. 5. Because development studies observed lower false positive assay noise for the ddPCR assay (average <0.1% methylation) compared to MassARRAY (average 4.5%), qualitative call thresholds were adjusted for ddPCR data interpretation according to FIG. 14.

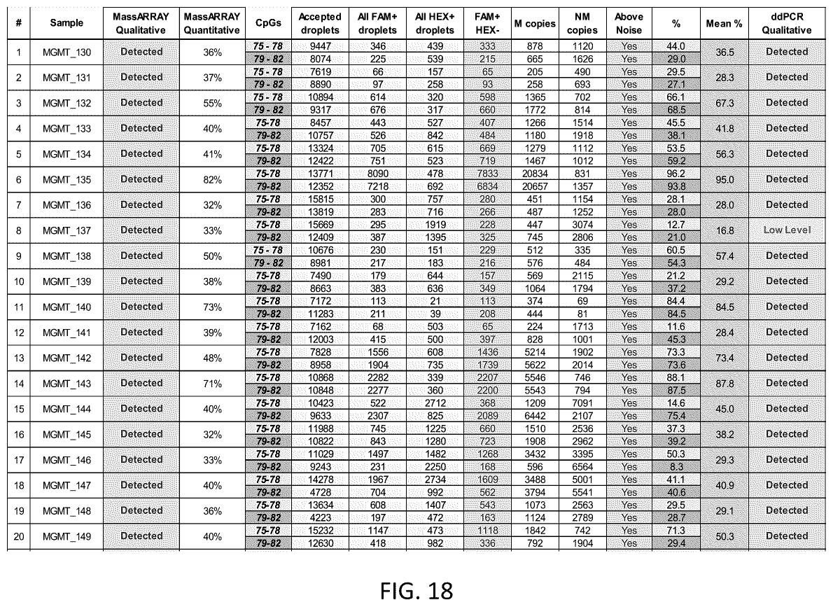

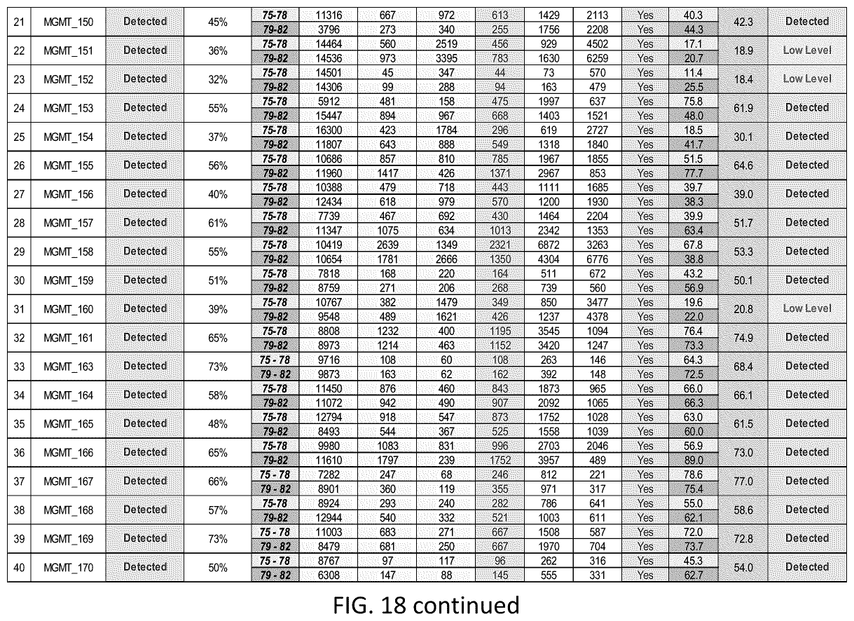

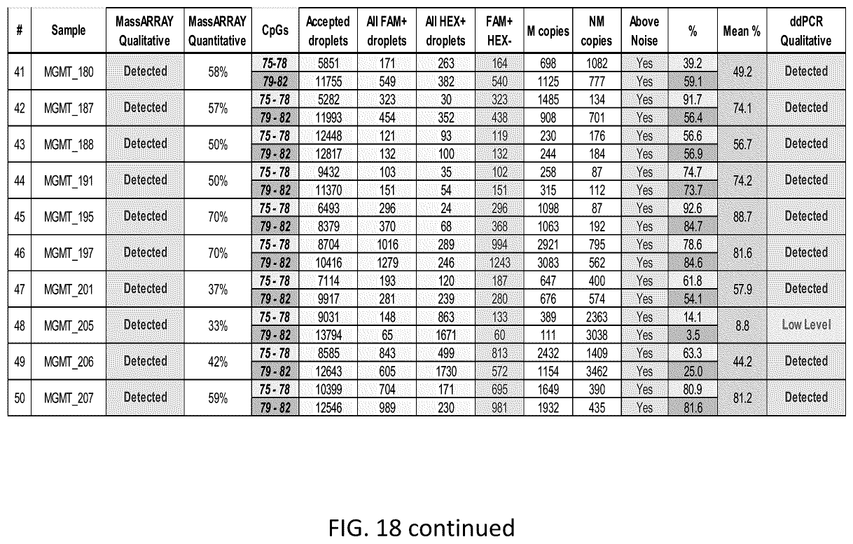

50 patient samples Detected by MassARRAY (% methylation ≥30% by MA) were tested by ddPCR (FIG. 18). No disqualifying QC failures were observed for this cohort. All samples returned raw FAM+/HEX− droplet counts above assay noise in both test wells. Qualitative thresholding of observed mean percent methylation resulted in Detected outcomes for 45 (90%) of the patient samples. The remaining five samples returned Low Level positive results, with detected percent methylation ranging from 8.8 to 20.8%. Because the qualitative distinction between Low Level and Detected percent methylation is largely arbitrary with unclear clinical significance, samples that returned Low Level outcomes by ddPCR but Detected outcomes by MassARRAY are not considered discordant in the context of this test validation.

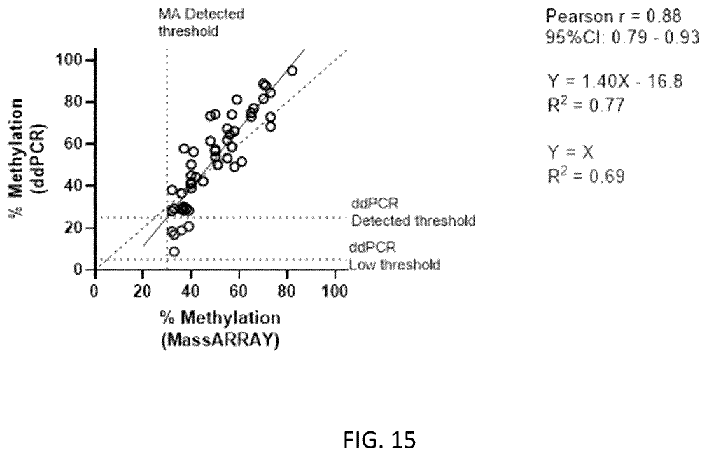

Comparison of quantitative outcomes showed very good correlation between percent methylation detected by MassARRAY and ddPCR methodologies in this cohort (FIG. 15, Pearson r=0.88, P<0.0001).

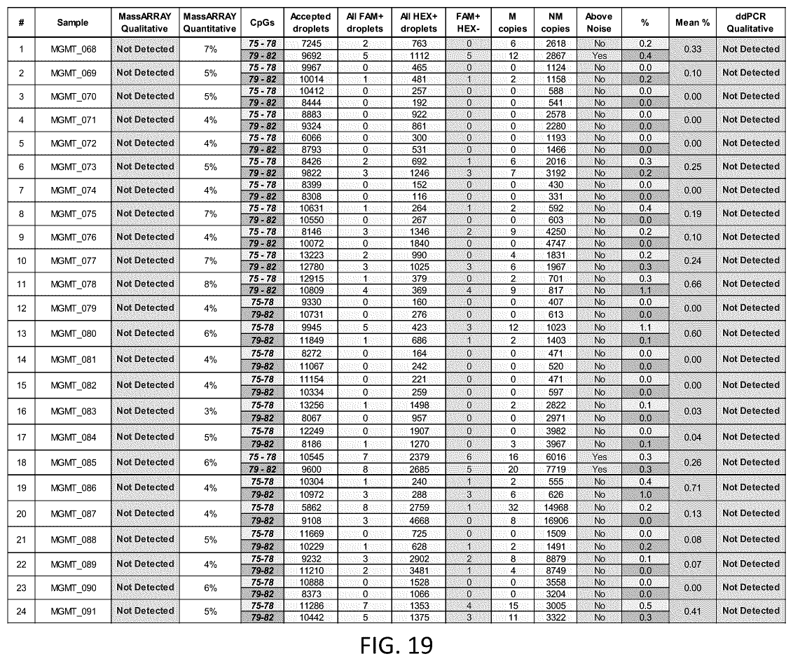

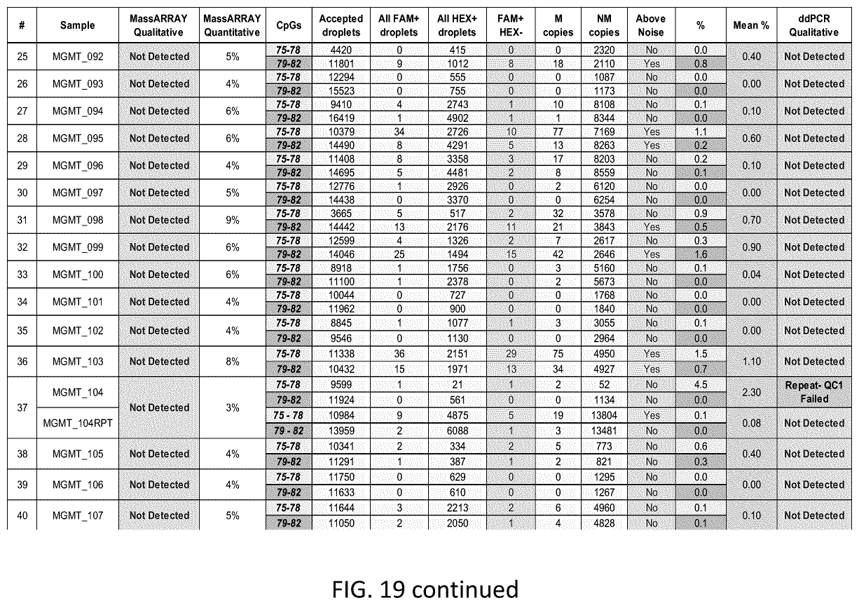

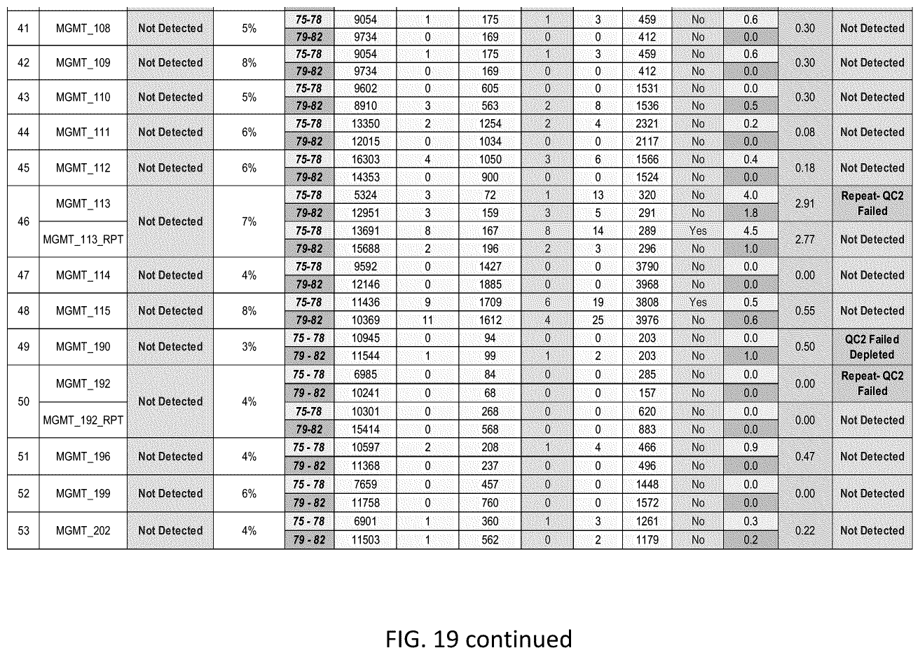

53 patient samples that were resulted as Not Detected by MassARRAY (methylation <10%) were tested by ddPCR (FIG. 19). Four samples (MGMT_104, 113, 190, 192) resulted in QC failures on initial testing. Three samples passed QC on repeat testing. One sample (MGMT_190) could not be repeated due to sample depletion and was excluded from further accuracy analysis.

Of the remaining 52 samples, 41 (78.8%) resulted in raw FAM+/HEX− droplet counts within the assay noise, resulting in a Not Detected outcome. Eleven samples (21.2%) had above noise FAM+/HEX− droplet counts in one (seven samples) or both assay wells (four samples) but were resulted as Not Detected based on observed percent methylation values below the clinical significance threshold of 5%.

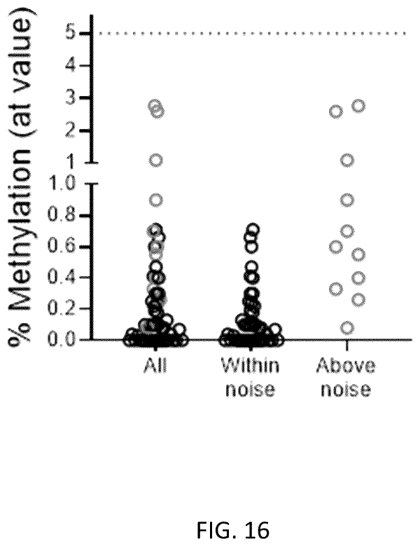

Average percent methylation at value was higher in the subset of patient samples with above noise raw droplet counts in at least one well (mean of 0.94%) compared to samples entirely within the assay noise (mean of 0.14%), but there was considerable overlap the range of percent methylation observed for both sample subsets (within noise: 0-0.71%; above noise: 0.08-2.8%; FIG. 16). This is likely predominantly due to the variability in ddPCR template input that results from size differences in the extracted tumor tissue area and varying degrees of efficiency in the bisulfite conversion process. Equal counts of raw data noise ultimately correlate with a range of at-value percent methylation in samples with different overall amplifiable MGMT copy numbers.

Samples with above noise FAM+/HEX− droplet counts are very likely to contain a small number of highly methylated MGMT promoter copies. However, at the time of validation the clinical significance of very low (<5%) MGMT promoter methylation as detectable by ddPCR remains unclear.

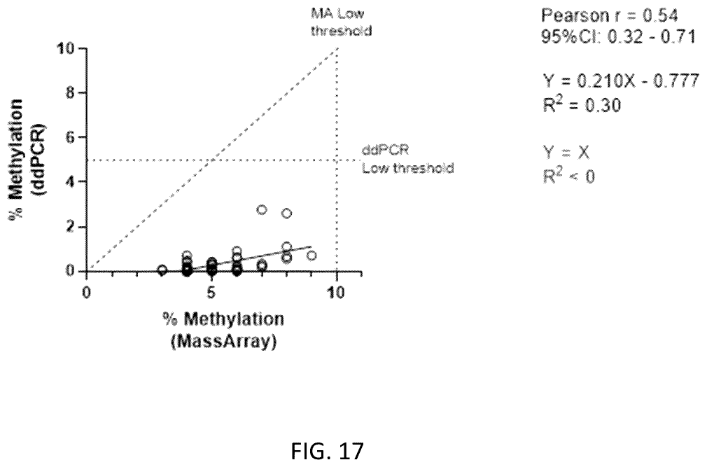

Quantitative correlation between MassARRAY and ddPCR methylation percentages in the Not Detected sample group was overall poor (FIG. 17). As observed during assay development studies, in the cohort of 52 Not Detected patient samples, MassARRAY resulted in higher at-value percentages of methylation with a mean of 5.2% (range 3-9%) compared to ddPCR with a mean of 0.31% (range 0-2.77%).

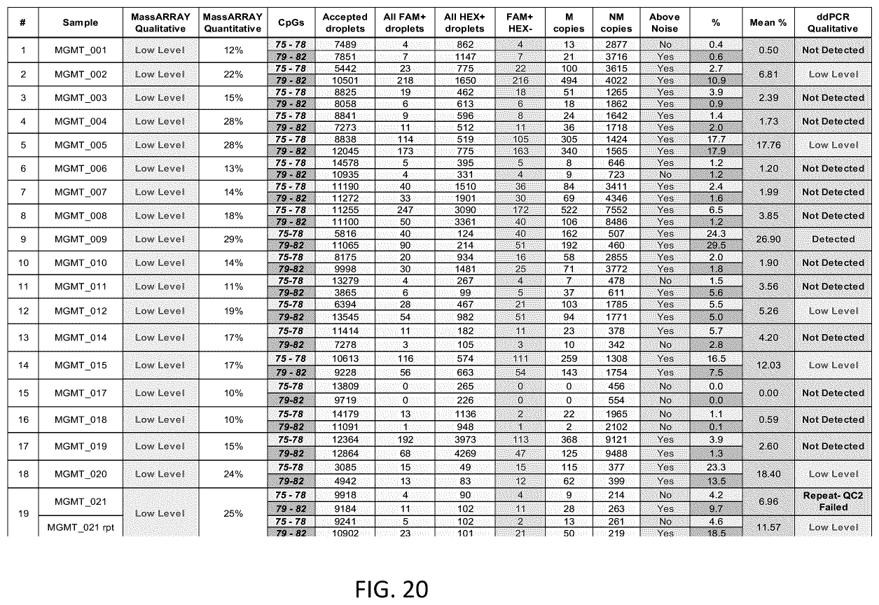

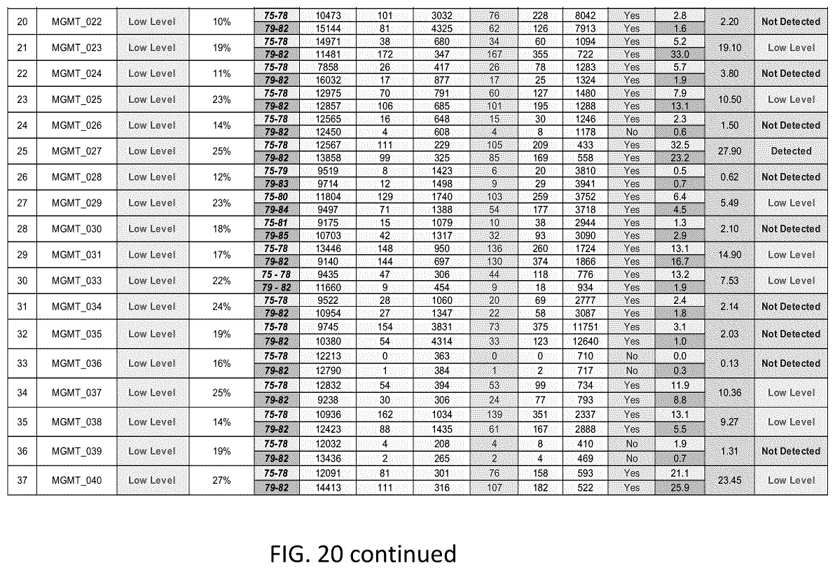

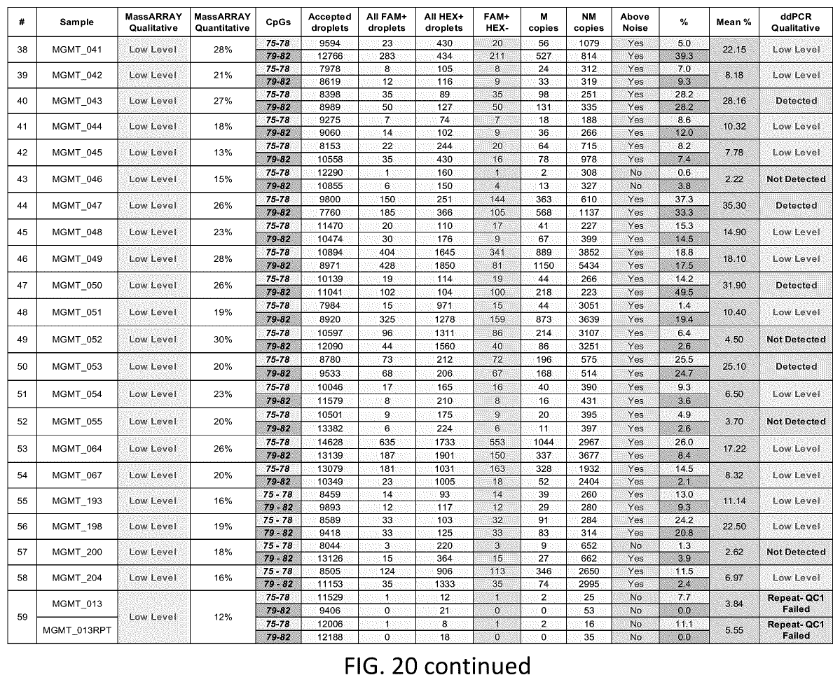

Fifty-nine patient samples resulted as Low positive (10-30% methylated) by MassARRAY were tested by ddPCR (FIG. 20).

One sample (MGMT_013) failed QC thresholds in initial and repeat testing and was excluded from the accuracy cohort. One additional sample (MGMT_021) failed QC in initial testing but passed checkpoints in repeat testing.

Twenty-seven of the remaining 58 samples also resulted in Low Positive qualitative calls by ddPCR (5-25% methylation). Six samples yielded Detected qualitative calls by ddPCR (≥25% methylation).

Twenty-five samples had apparent discordant outcomes in returning a Not Detected qualitative outcome by ddPCR. Of those, five samples scored raw droplet counts within the noise window in both wells, resulting in Not Detected call. Twenty samples were above noise in one (6) or both (14) wells but resulted in Not Detected calls based on observed mean methylation levels <5%. To explore and potentially resolve apparent discrepant results two complementing approaches were taken: 1) Deidentified MassARRAY reports of discordant samples were consulted to assess percent methylation and resulting qualitative outcome after exclusion of data in the “short amplicon right” region (SAR, CpGs 86-89). The SAR region was excluded from the MassARRAY clinical test in July 2021, with the remaining queried CpGs 72-83 more closely overlapping with the ddPCR methylation analysis coverage. 2) Twenty-three of the 25 discordant samples were tested by pyrosequencing, which provides single-CpG methylation level resolution for CpGs 74-83. In accordance with the consensus of published literature, MGMT methylation by pyrosequencing <10% was considered Not Detected by virtue of falling within the assay noise and/or lacking clinical significance. For two discordant samples (MGMT_052 and MGMT_055) pyrosequencing data was not obtained.

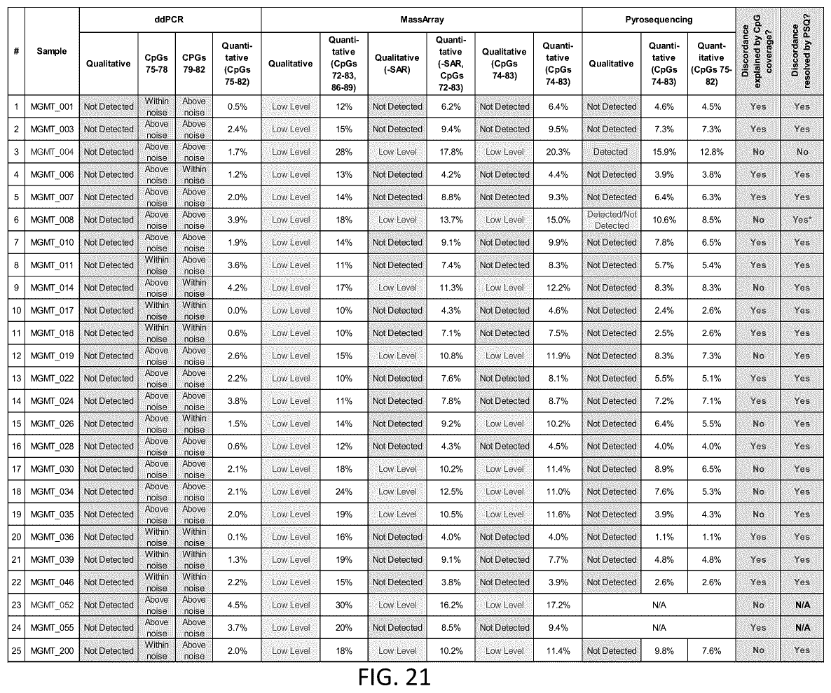

A summary of the effort to consolidate discordant findings is provided in FIG. 21.

For 16 of the discordant samples limiting the CpG coverage of the MassARRAY assay to CpGs 72-83 explained the qualitative discrepancy. For those samples the Low Level clinical reporting can largely be attributed to the high methylation levels observed in the “SAR” region (CpGs 86-89), which is not interrogated by the ddPCR assay. For 15 of the 16 samples the Not Detected qualitative could further be confirmed by orthogonal pyrosequencing analysis. Pyrosequencing data was not obtained for one sample (MGMT_055).

For seven of the remaining nine discrepancies using Pyrosequencing as an orthogonal testing method (tiebreaker) produced results that supported the qualitative outcome of the ddPCR method.

One discordant case, MGMT_004, could not be resolved by adjusting CpG coverage of the MassARRAY or orthogonal testing. For one additional case, MGMT_052, tie-breaking pyrosequencing results were not obtained.

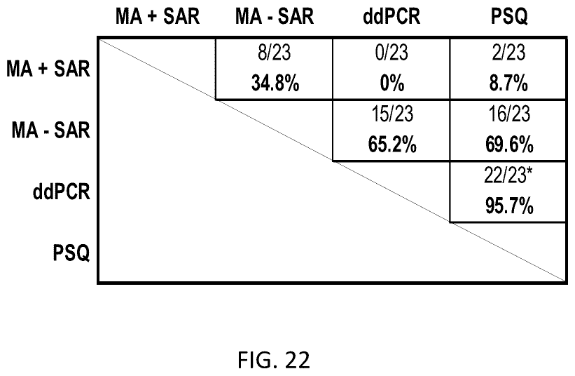

In the dataset of 23 discordant low samples for which pyrosequencing data was available, qualitative call concordance was highest (95.7%) between ddPCR and pyrosequencing results (FIG. 22).

Restricting CpG coverage to largely overlapping regions improved the qualitative concordance between MassARRAY and the alternate testing methods significantly (65% improvement for ddPCR, 60.9% improvement for pyrosequencing), but did not fully account for differences. Baseline assay noise is expected to contribute to quantitative results and may therefore influence qualitative calls. When comparing quantitative data obtained for the nonmethylated standard in-run control (Human HCT116 DKO Nonmethylated DNA; Zymo), at value percent methylation was lowest for ddPCR (range 0-0.1%), increased in pyrosequencing (Range 1.4-2.3%) and highest and most variable in MassARRAY tests (range 2.8-4.8%) (FIG. 23A). Ten randomly chosen qualitatively concordant Not Detected patient samples (i.e. Not Detected by MassARRAY, ddPCR and pyrosequencing) produced qualitative data that closely reflected the results of the nonmethylated commercial standard (FIG. 23B).

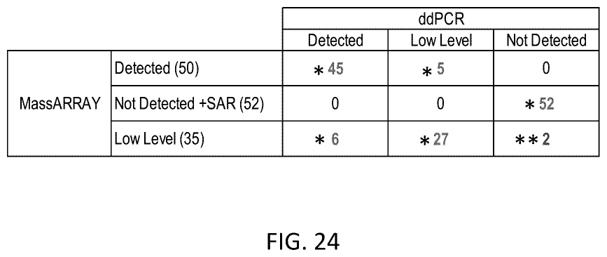

In summary, patient-sample based Accuracy studies performed as part of this validation produced the following results (FIG. 24). Of 50 samples with MGMT methylation Detected by MassARRAY 45 were Detected by ddPCR, 5 were Low Level positive by ddPCR (considered not discordant). Of 52 samples with MGMT methylation Not Detected MassARRAY; 52 had Not Detected outcomes by ddPCR based on within noise data signal or methylation levels <5%. Of 58 samples with MGMT methylation detected at Low Level by MassARRAY; 27 had Low Level methylation outcomes by ddPCR; 6 had Detected methylation by ddPCR (considered not discordant); 16 had Not Detected outcomes by ddPCR that were shown to be a result of changes in CpG coverage of the ddPCR assay compared to MassARRAY. These samples were excluded from final calculation of overall and positive percent agreement in the accuracy cohort because MGMT methylation was outside the CpG range covered by the ddPCR assay. 7 had Not Detected outcomes that were supported by pyrosequencing results across the ddPCR CpG coverage region. These samples were excluded from final calculation of overall and positive percent agreement in the accuracy cohort because the accuracy of the original MassARRAY result was disputed by quantitative pyrosequencing. 2 samples showed qualitative discordance was not resolved.

Overall percent agreement was observed for 135/137 samples, corresponding to 98.5% (95% CI: 95.4-99.7%, Jeffreys approximation).

Positive percent agreement (PPA) was observed for 83/85 Detected or Low Level patient samples, corresponding to 97.7% (95% CI: 92.7-99.5%, Jeffreys approximation).

Negative percent agreement (NPA) was observed for 52/52 Not Detected patient samples, corresponding to 100% (95% CI: 95.3-100%, Jeffreys approximation).

### 6. Validation of Assay Linearity

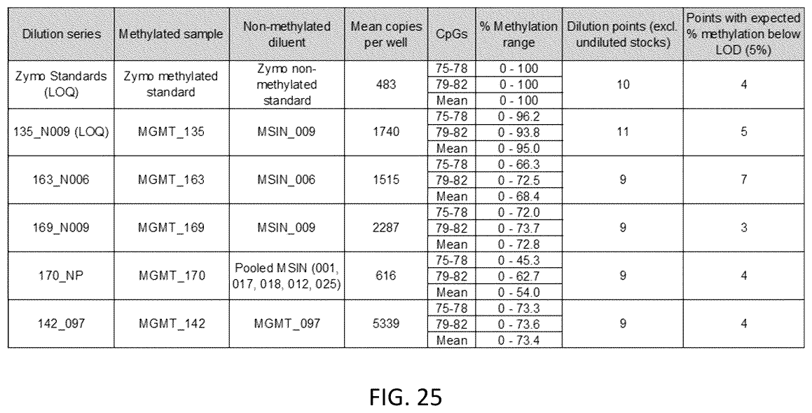

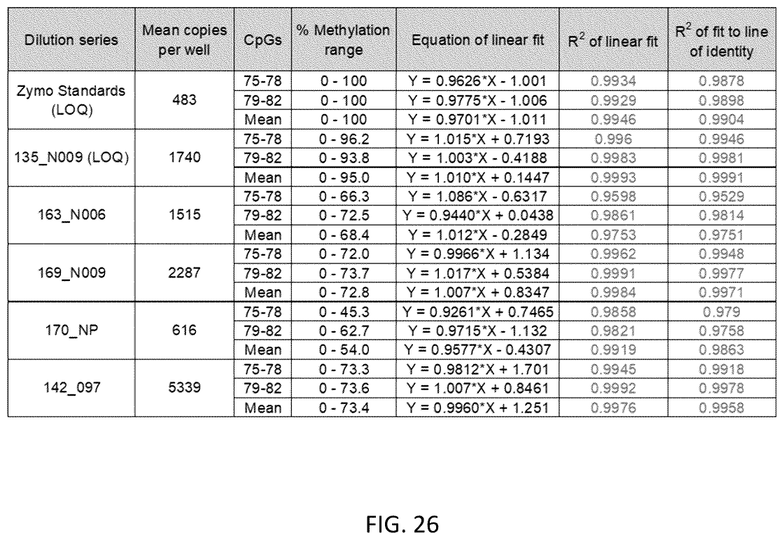

Linearity of the assay was tested using MGMT promoter methylation dilution series generated from Zymo methylation standards and from five MGMT patient samples. Dilution series were prepared by mixing methylation-positive samples with copy number matched non-methylated diluents. Expected percent methylation of each dilution point were calculated from applied mix ratios and detected methylated and nonmethylated copies of undiluted mixing partners included in the same run. The six dilution series were generated to reflect a range of total MGMT copy numbers per well. Each series contained a minimum of nine dilution points, including a minimum of three points below the validated target LOD of 5% methylation. An overview of the dilution series included in the linearity analysis is provided in FIG. 25.

Due to limited patient sample volumes, dilution points were prepared and tested in single replicates for four series. To evaluate the LOQ of the assay (see below), two series were prepared and tested as within-run triplicates for all dilution points. To test linearity, observed percent methylation for each group of CpGs and the mean percent methylation were plotted against the calculated expected values over the full range of the dilution series. Data were fit by linear regression and the R² value of the fit was determined. R² was also determined for the fit to the line of identity.

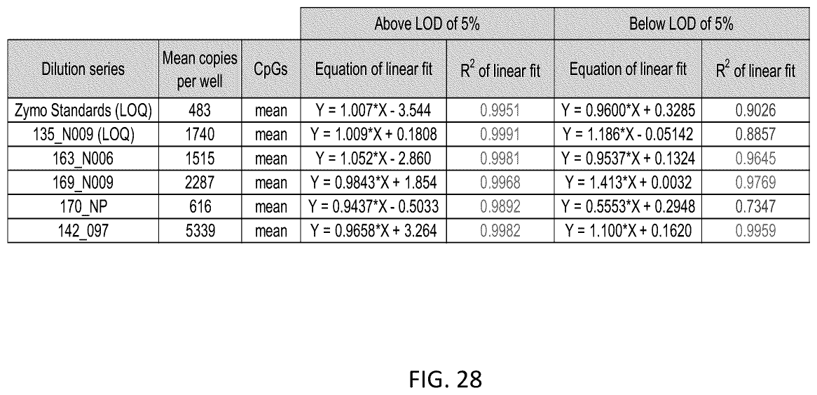

R²>0.95 was observed for the linear regression of all analyzed dilution series. Linearity was observed in both CpG groups as well as the mean of both assay wells. When dilution points above and below the validated LOD of 5% methylation were analyzed for linearity separately, R²>0.95 above the LOD was observed for all dilution series (FIG. 28). Good fit to linear trends below the LOD was also observed for a subset of dilution series.

Quantitative results of the assay are linear above the target LOD of 5%.

### 7. Validation of Analytical Sensitivity/Limit of Quantification/Limit of Detection

The dilution series generated for the validation of assay linearity were also utilized to validate the target assay LOD of 5% methylation (mean methylation over CpGs 75-82). Counting all replicates, the dilution series contained a total of 71 data points with expected percent methylation above and 58 data points below the assay's target LOD of 5%. Three data points, one above and two below the LOD, were excluded because the well data was insufficient to meet QC requirements. One additional data point (Dilution 3 of the 170_NP series) was excluded due to abnormal droplet clustering with absent HEX−/FAM− counts.

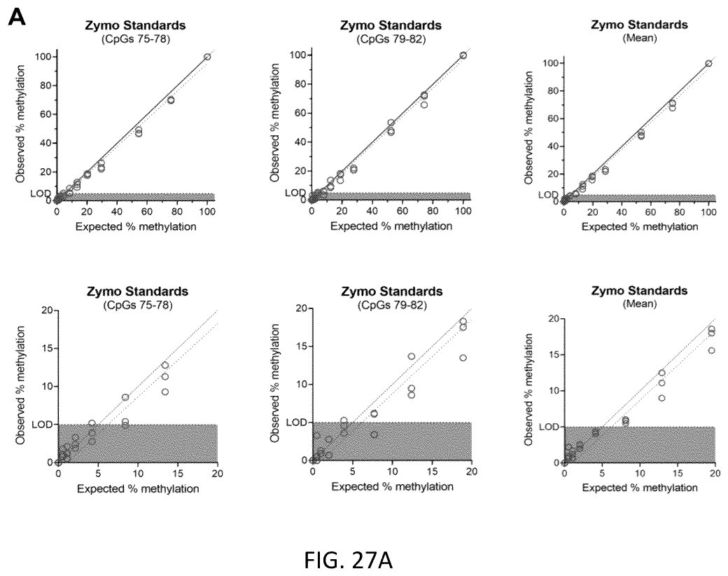

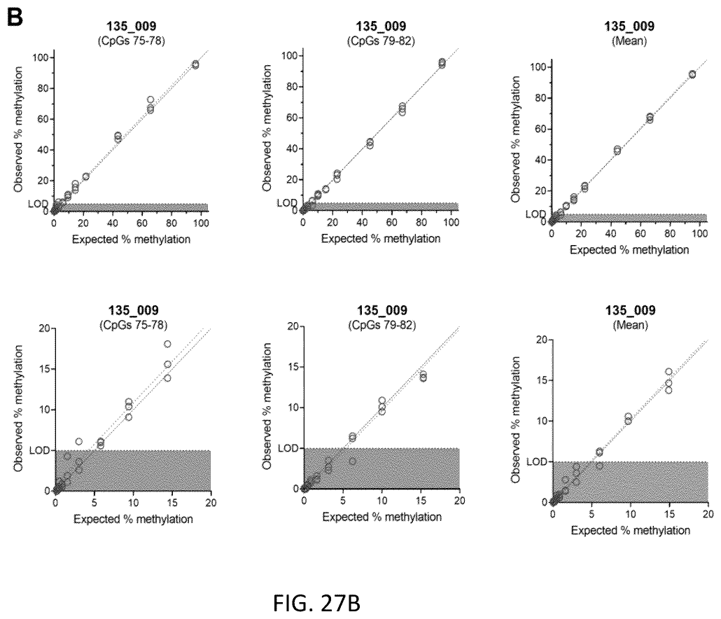

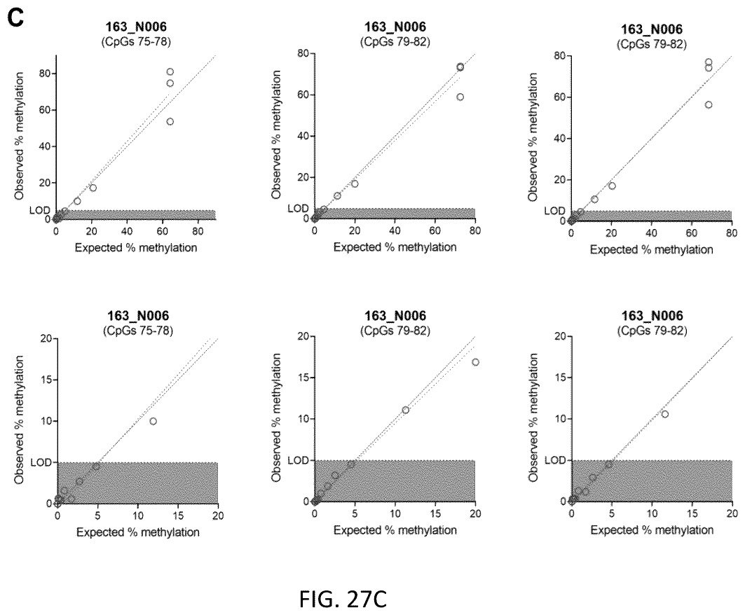

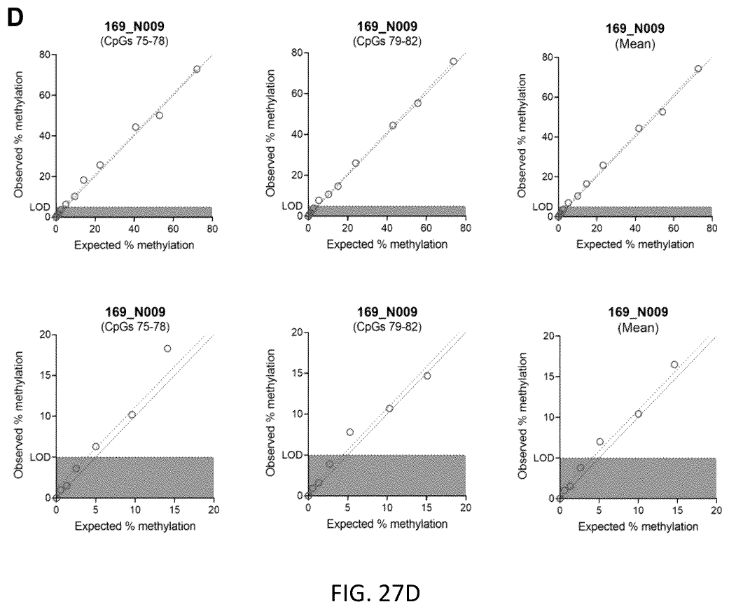

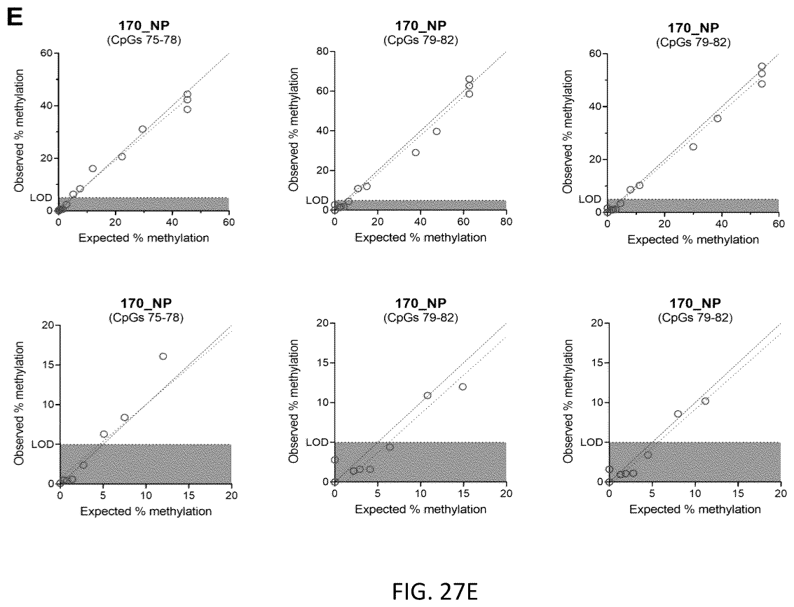

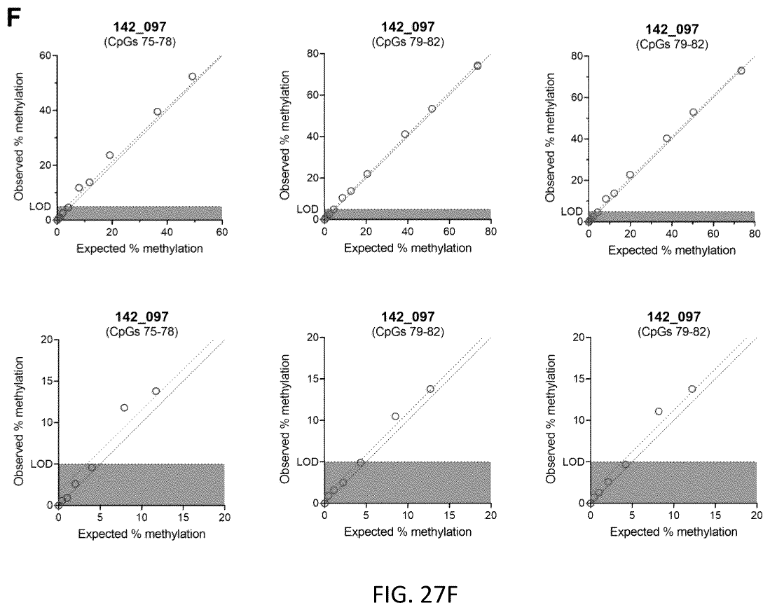

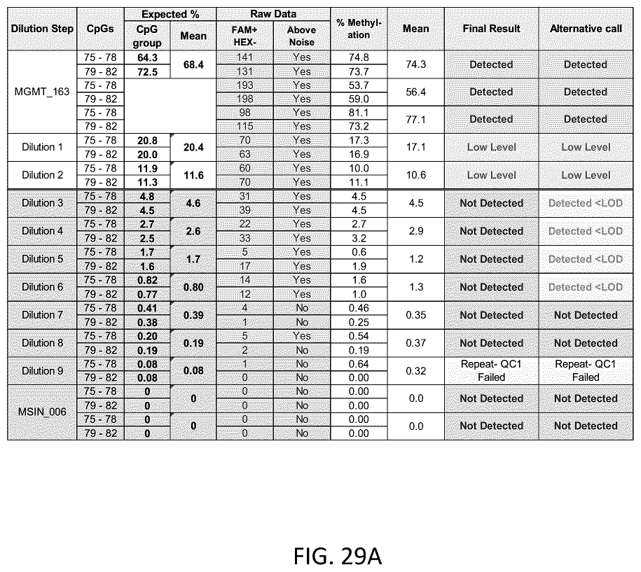

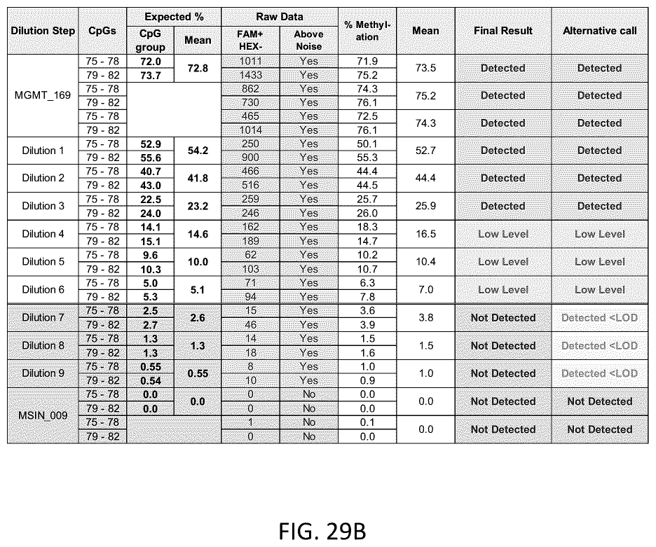

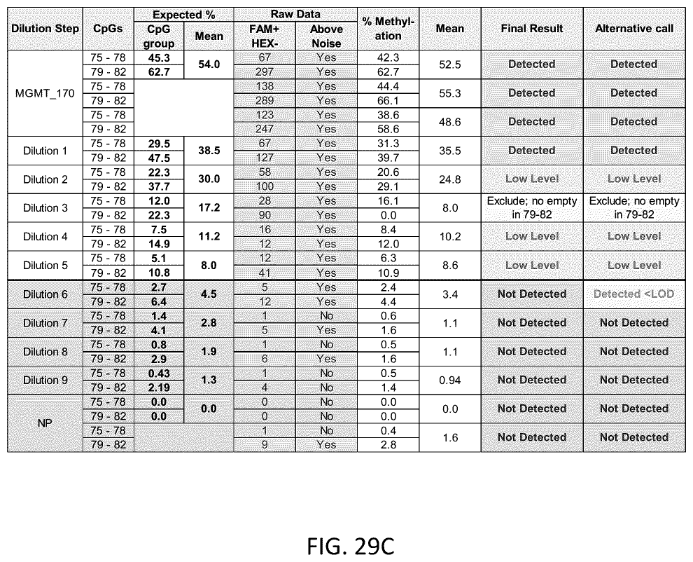

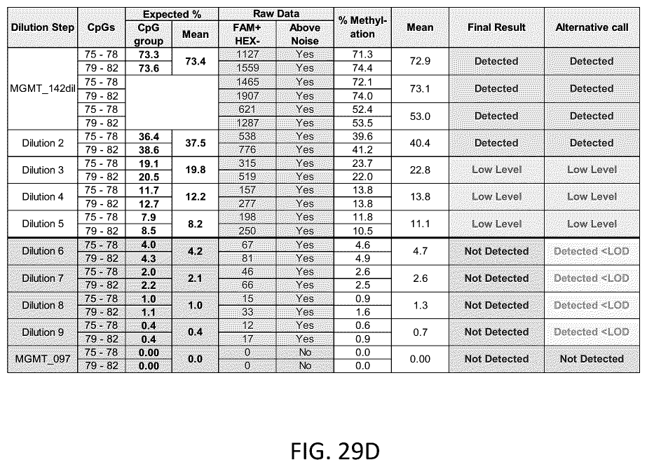

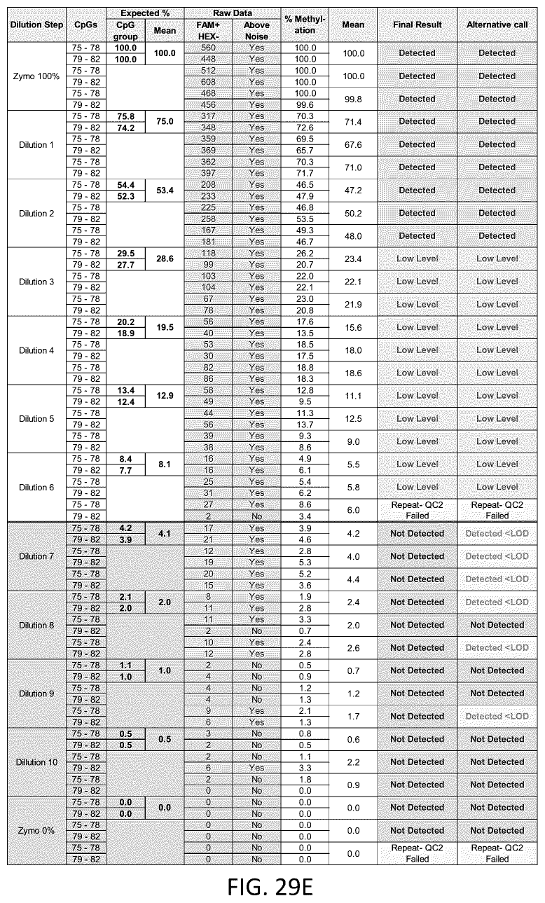

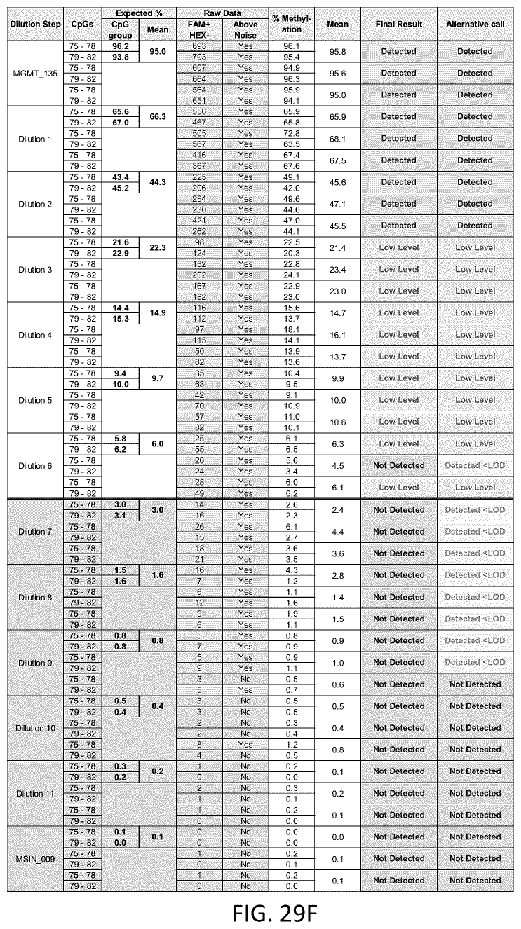

Data are summarized in FIG. 29A-F below and FIGS. 27A-F.

Of the 69 data points with expected mean percent methylation at or above 5%, 68 (98.6%; 95% CI: 93.4-99.8%, Jeffreys approximation) returned Low Level or Detected outcomes The remaining data point (135 N009, Dilution 6, replicate 2) showed a mean percent methylation <5%. However, raw droplet counts in the FAM+/HEX− raw data quadrant, were scored well outside the assay noise (20 and 24 counts for CpGs 75-78 and 79-82, respectively), enabling an alternative call as Detected <LOD (Validation purpose only; not applicable to clinical testing).

Of the 56 data points with expected mean methylation below the LOD, 56 (100%, 95% CI: 95.6-100%, Jeffreys approximation) returned methylation levels <5%, resulting in Not Detected outcomes. 26 of the 56 samples scored raw droplet counts in the FAM+/HEX− raw data quadrant outside the assay noise in both assay wells.

While quantitative data will not be reported in clinical testing, the LOQ of the assay was determined for internal purposes. To establish the Limit of Quantification (LOQ) of the assay two dilution series were tested in triplicate (within-run) for each dilution point. The dilution series were created from a commercial cell-line based standard at low converted copy concentration (<500/well) and from a methylation positive patient sample at a converted copy concentration close to the median of all accuracy samples tested (˜1740/well in MGMT_135/MSIN_009 series compared to median of 1883/well in accuracy cohort).

CLSI defines the LOQ as the lowest amount of measurand in a sample that can be quantitatively determined with stated, acceptable precision and stated, acceptable accuracy. While there is no general guidance specifying acceptable threshold values of LOQ, in the context of this validation the LOQ was defined as the percent methylation (average over CpGs 75-82) that can be determined with a precision of CV≤30% and an accuracy of CV≤30% from the mean of the expected value. In the context of LOQ validation, accuracy will be defined as agreement with calculated percent methylation expected for each dilution point. The assays LOQ may be identical or distinct from the validated LOD.

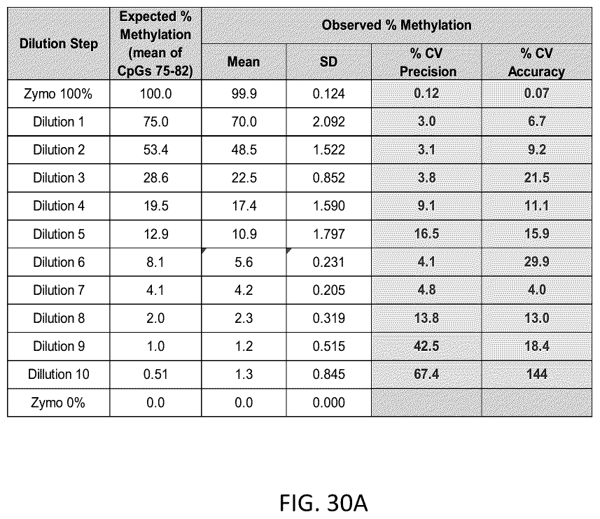

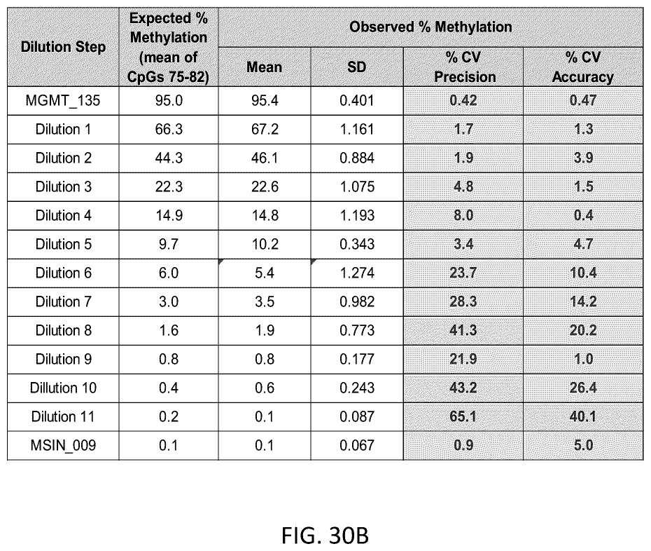

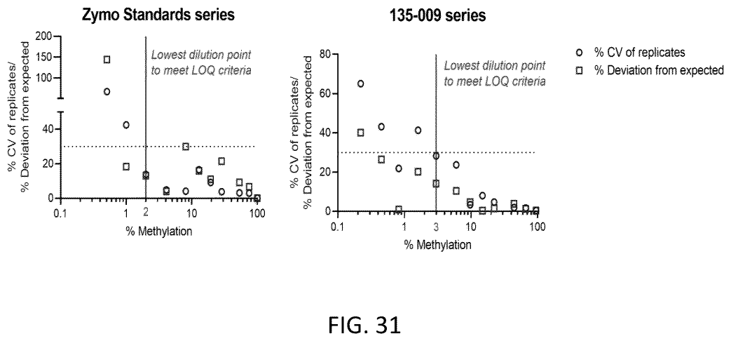

FIGS. 30A-B and FIG. 31 provide an overview of the relative dispersion of quantitative methylation measurements observed for replicate data points relative to their means (i.e. % CV Precision) and relative to the expected value calculated from mix ratios (i.e. % CV Accuracy). LOQ requirements for each dilution point are met when both % CV Precision and % CV Accuracy are ≤30.

### 8. Validation of Precision

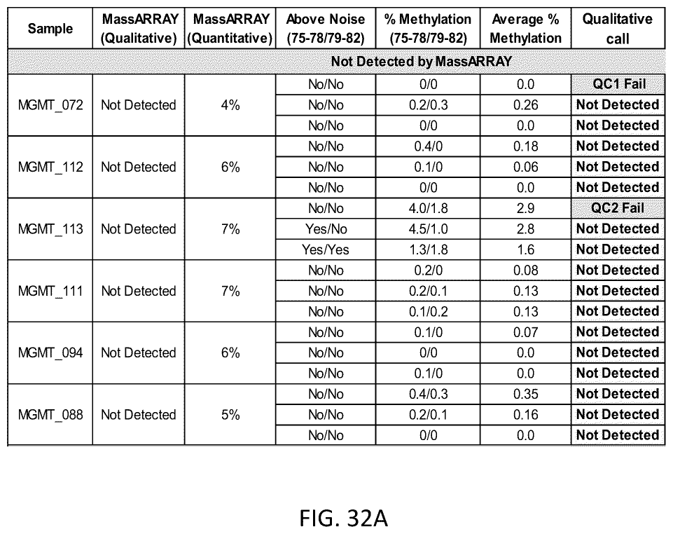

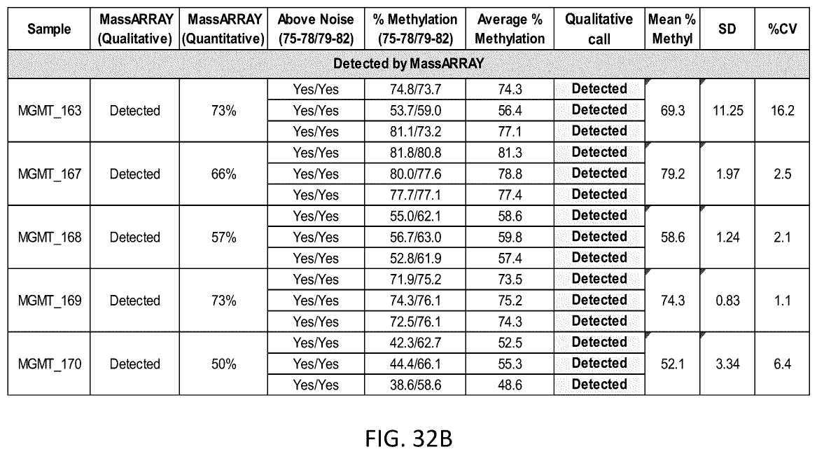

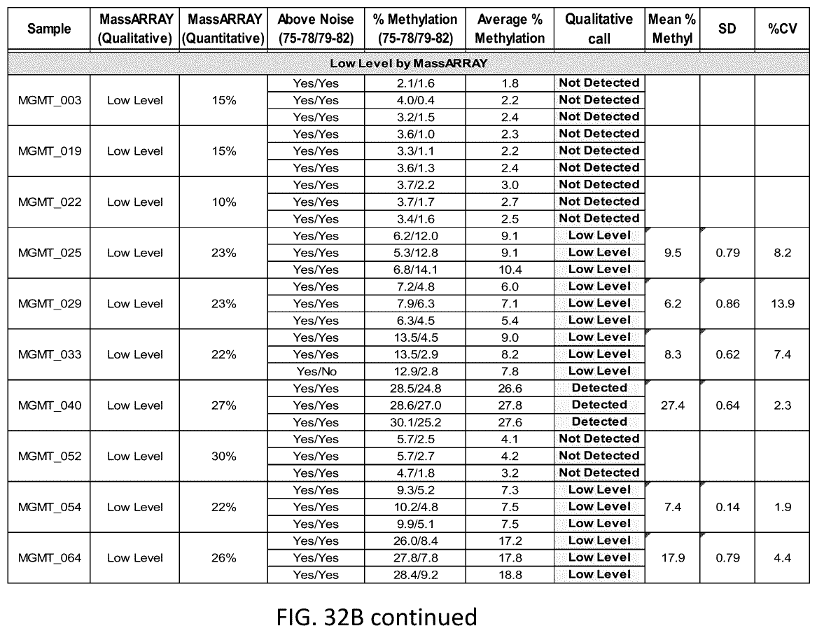

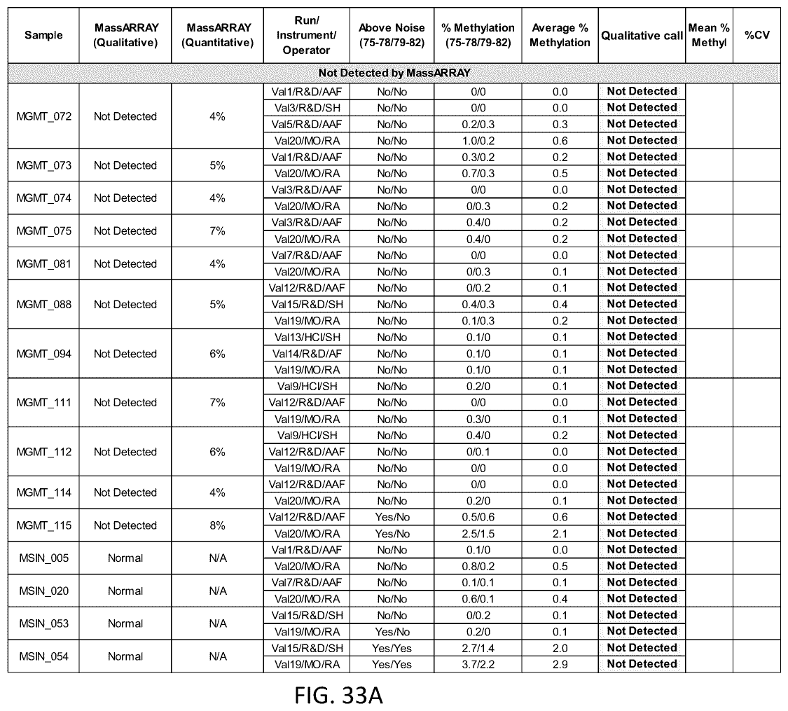

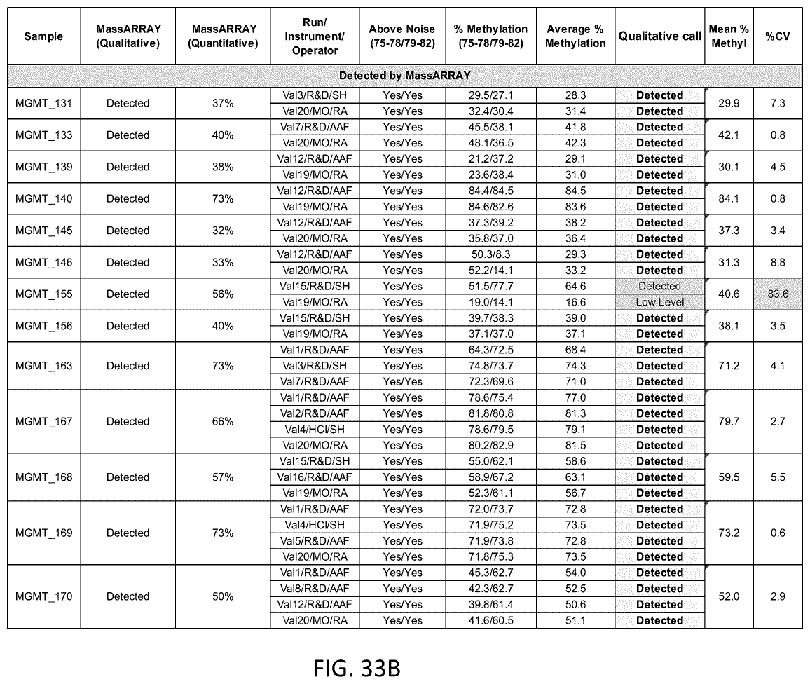

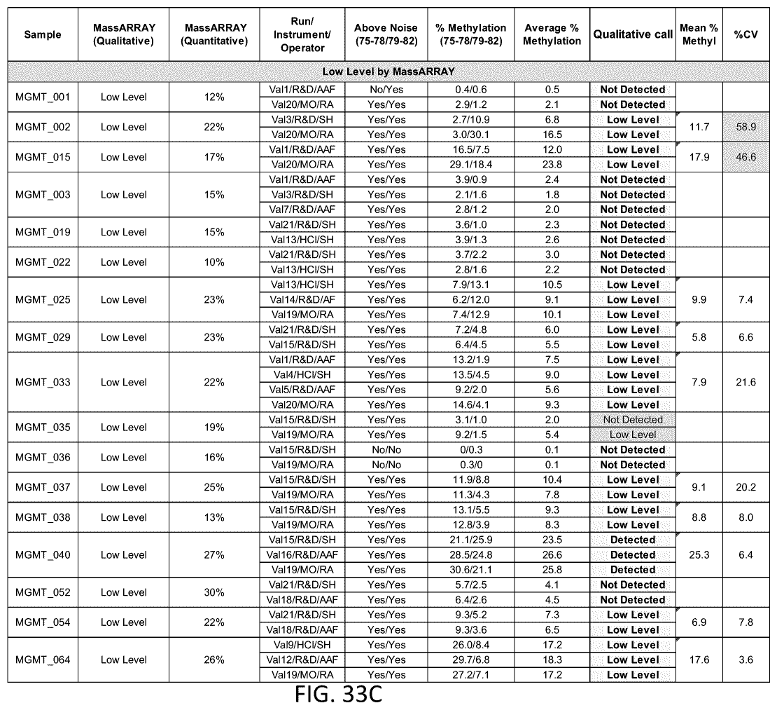

Repeatability (within-run precision) and reproducibility (between-run precision) was validated using standards and patient samples. Detailed run information data are summarized in FIGS. 32A-B and FIGS. 33A-C below.

i. Precision of Patient Sample Testing

For repeatability studies a total of 21 patient samples were tested in three within run replicates each. Replicates were bisulfite converted replicate wells on the same conversion date and subsequently tested by ddPCR. Of the 21 patient samples, six were reported as Not Detected, five as Detected and ten as Low Level by MassARRAY.

Of the Not Detected sample group two samples failed quality control checkpoints in one replicate well (FIG. 32A). All remaining 16 repeatability data points were in qualitative agreement. Quantitative comparisons were not made on account of Not Detected qualitative calls by ddPCR.

An overlapping but not identical set of 45 patient samples was utilized for validation of between run precision. These included four MSI normal samples tested in the specificity cohort study and 39 MGMT clinical specimens with 11 samples Not Detected, 13 samples Detected and 17 samples with Low Level methylation by MassARRAY. Between two and four between run replicates were tested for each sample, with bisulfite conversions and subsequent ddPCR occurring on different dates. Whenever possible different operators performed replicate experiments. Inter-instrument repeatability was tested as part of this study, with particular focus on comparison between the “MolOnc” and “R&D” instrumentation which are used as the primary and backup instrumentation for clinical testing, respectively. Thirty-eight of the 43 samples included a replicate performed by a clinical lab operator on the “MolOnc” instrumentation.

Of the MSI normal and Not Detected sample group, all 36 replicate data points from 15 samples (10 duplicates, 4 triplicates, 1 quadruplicate) were qualitatively in agreement with Not Detected outcomes by ddPCR (FIG. 33A).

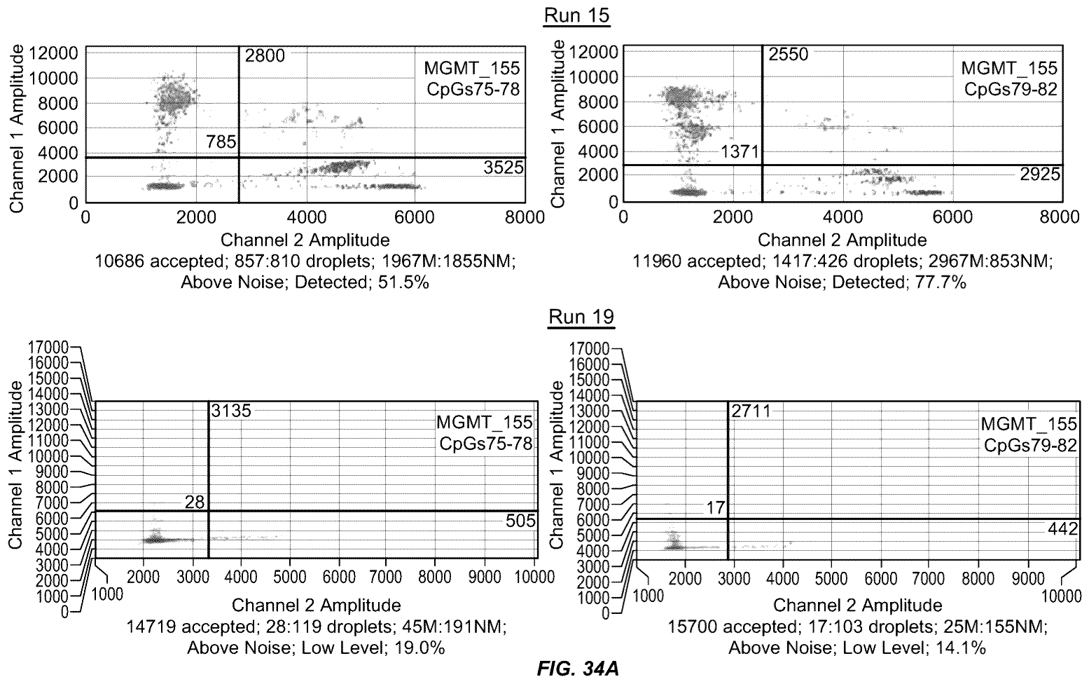

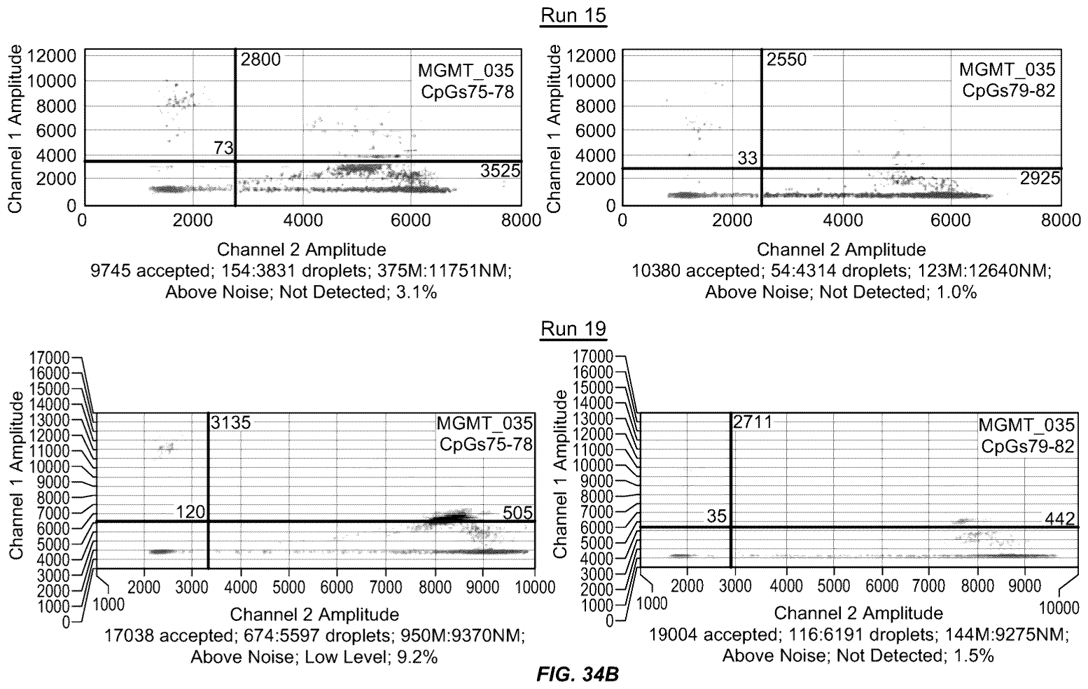

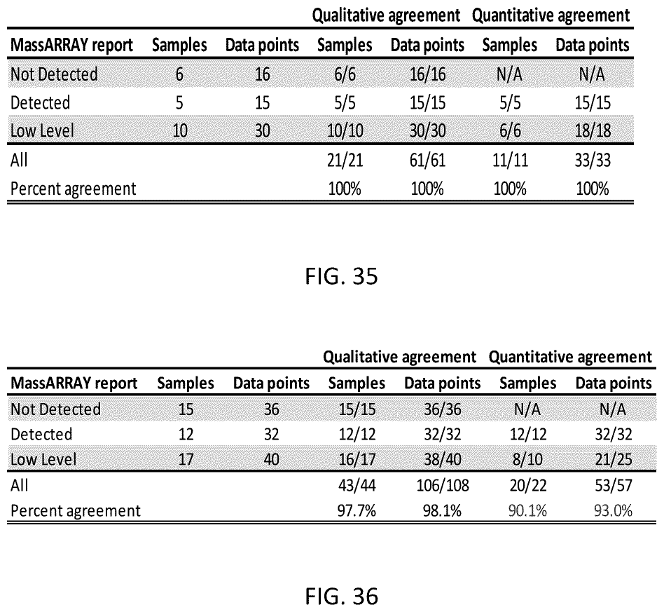

Among the 34 between run replicates of samples Detected by MassARRAY (8 duplicates, 2 triplicates, 3 quadruplicates), 32 were qualitatively in agreement (FIG. 33B). For sample MGMT_155 one replicate produced a Low Level outcome at 16.6% average methylation, compared to the 64% methylation observed in an independent run. Inspection of the raw data plots of the MGMT_155 replicates (FIG. 34A) revealed strong evidence of EtOH carry-over in the second replicate. As discussed in the Assay Interference section, ethanol contamination due to incomplete elimination of wash buffer during the bisulfite conversion procedure negatively affected ddPCR signal amplitude and measurable total copy number for MGMT_155 replicate 2. Because the process failure largely prevents direct comparison of ddPCR results for the sample, MGMT_155 was excluded from the between-run precision evaluation.

Quantitative agreement was evaluated within in each replicate set, with an acceptability limit of 20% CV defined by the validation plan. Observed % CV ranged from 0.6 to 8.8% (mean of 3.7%).

Seventeen samples with Low Level methylation levels by MassARRAY were tested in a total of 40 between-run replicates (12 duplicates, 4 triplicates, 1 quadruplicate, FIG. 33C). Qualitative agreement was observed for 38 replicates. While showing above noise raw data in both wells for each of the replicates, differences in the observed percent methylation resulted in discordant Not Detected and Low Level calls. Inspection of the raw data plots showed no discernible differences in the overall data quality (FIG. 34B).

For Low Level and Detected outcomes quantitative agreement was assessed. Coefficients Qualitative outcomes within each replicate group were within the acceptability limits of 20% CV for Detected outcomes. For 8/10 samples (21 replicate points) with Low Level qualitative calls the observed % CV fell within the predetermined acceptability limit of ≤40. Two Low Level samples, MGMT_002 and MGMT_015 showed quantitative replicate results >40%, at 58.9% and 46.6% CV respectively.

The observed precision of patient sample testing can be summarized as follows: 1) Within-run precision testing showed qualitative agreement of 61/61 replicates (21/21 samples), corresponding to 100% repeatability (95% CI: 96.0-100% for replicate data points and 88.9-100% for samples; Jeffreys approximation)—Quantitative agreement between replicates of Detected or Low Level outcomes was within acceptable ranges as defined by the validation plan: Detected outcomes: CV range 1.1%-16.2%; mean 5.1% (acceptable ≤20%); Low level outcomes: CV range 1.9%-13.9%; mean 7.2% (acceptable ≤40%). 2) Between-run precision showed qualitative agreement of 106/108 replicates (43/44 samples), corresponding to 98.2% (97.7%) reproducibility (95% CI: 94.2-99.6% for replicate data points and 89.9-99.8% for samples, Jeffreys approximation)—Quantitative agreement between replicates of 13 samples with Detected outcomes was within acceptable range of ≤20% as defined by the validation plan: CV range 0.6%-8.8%; mean 3.9%. —Quantitative agreement between replicates of 8 out of 10 samples with Low Level outcomes was within acceptable range of ≤40% as defined by the validation plan. Two samples (4 replicates) were quantitatively discordant with CVs of 58.9% and 46.6%. —Quantitative precision of in-between run replicates, therefore, deviated from the Validation Plan performance goals of ≥95% samples meeting precision limits (20/22 Det/Low samples, 90.1% CI 74.9-98.2%). However, the observed deviation was acceptable within the context of the collective Precision data set for the following reasons: The two samples with quantitative discrepancies returned concordant Low Level qualitative outcomes in each of the replicates. Because quantitative data is not reported during clinical testing, the observed discrepancies would not adversely affect results reporting or patient care; and the validation plan defined quantitative acceptability limits to be equal for within-run and between-run precision studies, based on observations during assay development. In hindsight, a more prudent strategy would have been to anticipate an increased level of variability for between-run measurements. Appropriate considerations should be made for future validation studies.

### 9. Validation of Analyte Stability

Analyte stability was assessed at two points of the sample handling process: after crude extraction of FFPE tissue according to MO-PROC-3028 with subsequent storage under refrigeration and after bisulfite conversion of samples with frozen storage of the conversion elution plate.

i. Stability of Crude DNA Extracts at 2-8° C.

Eleven slides were selected to validate stability after crude lysate extraction. Slides included six samples reported Detected by MassARRAY, two Low Level samples and three samples clinically reported as Not Detected. Tumor tissue was identified and circled by a pathologist, and slides were extracted according to MO-PROC-3028. The estimated area of extracted circled tumor tissue ranged from 40 to 225 mm2 (medium to extra-large). Extracted DNA was stored under refrigeration (2-8° C.). Within 24 hours of extraction, 20 μL of each sample was bisulfite converted and tested by ddPCR. A second aliquot was converted and tested after two weeks of storage, and a third aliquot was converted and tested after 4 weeks.

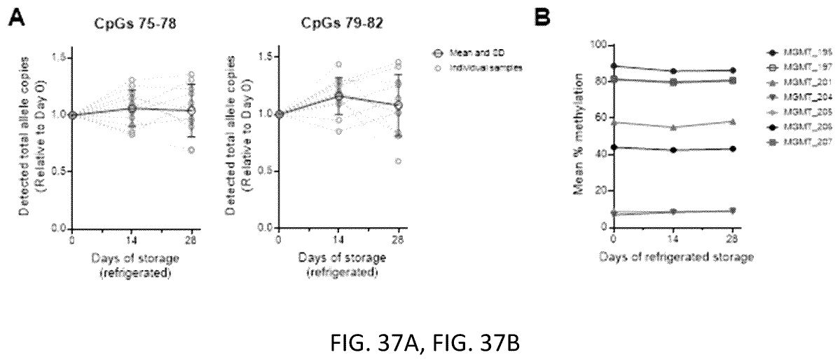

Data quality was evaluated by quantifying amplifiable total (methylated and nonmethylated) allele copy number in each assay well as a function of storage time. Consistency of qualitative calls was tracked over time as well as quantitative outcomes for Low Level and Detected samples. Concordance of qualitative and quantitative data with the MassARRAY results obtained for each sample is discussed in the Accuracy section of this validation. Detailed run data for the stability studies and findings are summarized in FIG. 37.

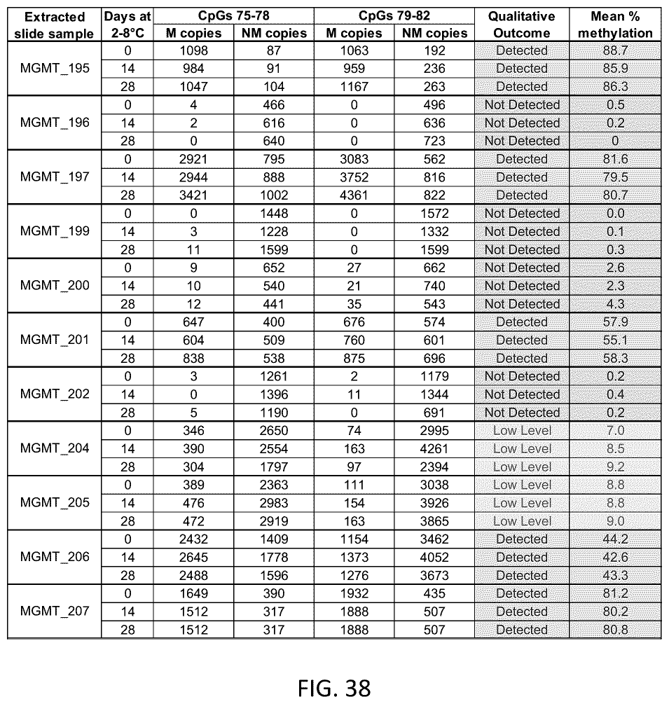

Total detected copy numbers ranged from ca. 450 to 5400 and was sample dependent (FIG. 38). For comparison purposes, the change in detected copy numbers relative to the Day 0 values was tracked (FIG. 37A). No relative loss was observed in amplifiable copy number in either test well over the 4-week test period. Qualitative outcomes for each sample were consistent over the test period and quantitative values for detected mean percent methylation in Detected and Low Level samples were was not adversely affected by two or four weeks of extract storage (FIG. 37B).

Up to four weeks of refrigerated storage of crude FFPE extracts does not adversely affect detection of MGMT methylation by the validated ddPCR method.

ii. Stability of Bisulfite Converted DNA after Storage Under Refrigeration or at −20° C.

To test stability after bisulfite conversion a set of 8 samples (100%, 20%, 10%, 0% controls and four patient samples) were converted in quadruplicate. Converted sample replicates were not pooled prior to storage and ddPCR. The conversion plate was stored under refrigeration and replicates were tested by ddPCR after 0, 3, 7 and 14 days of storage.

In a separate experiment the set of controls and four additional patient samples were converted and tested after plate storage under frozen (−20° C.) conditions.

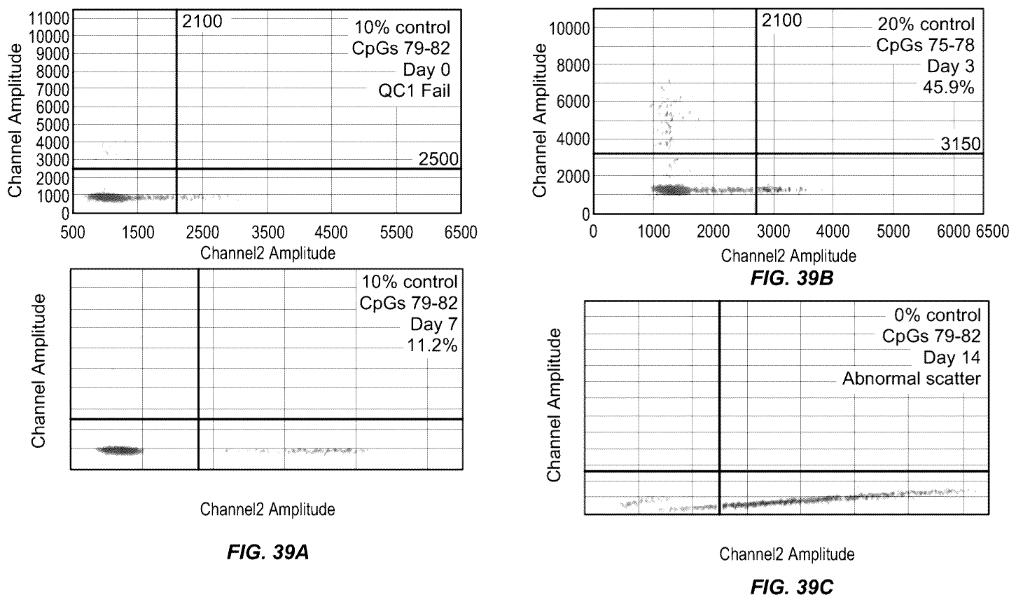

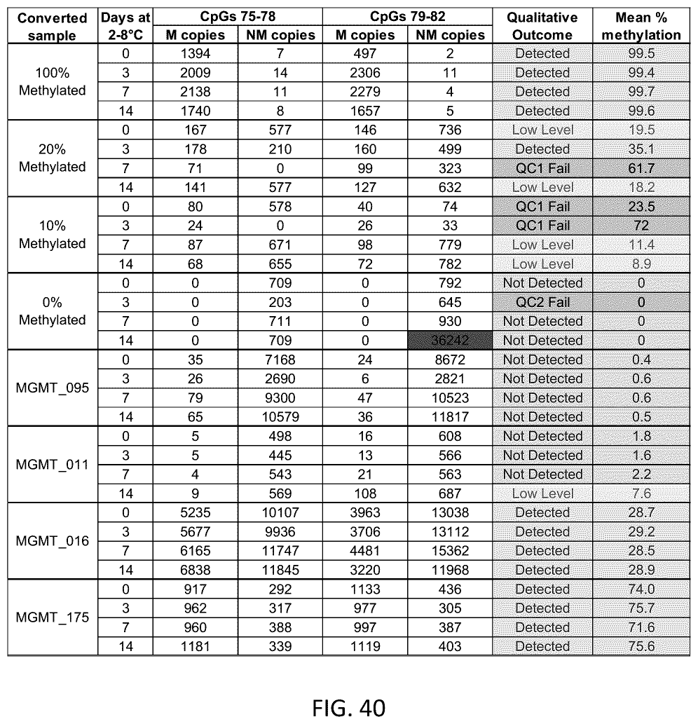

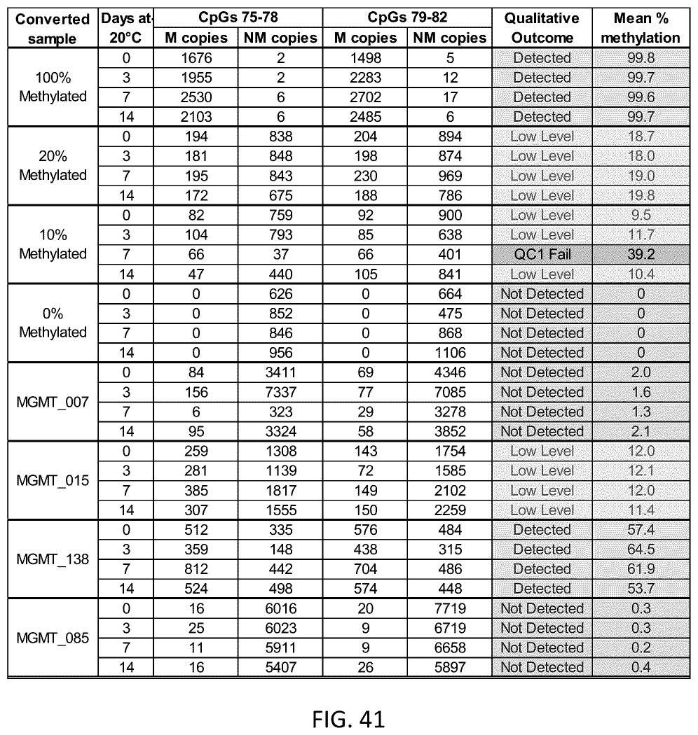

Data are summarized in FIGS. 39, 40, 41, and 42 below.

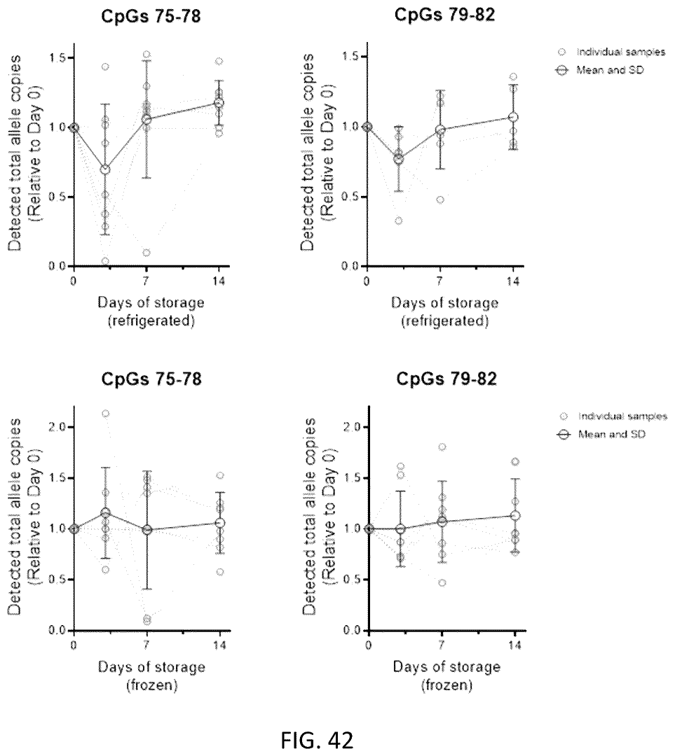

Data interpretation of the refrigerated post-conversion time course was complicated by evidence of ethanol contamination (discussed in section Testing of extrinsic interference by EtOH carry-over below) in select conversion wells that affected data quality but appeared unrelated to post-conversion storage time. Examples of affected data plots are shown on FIG. 39. In particular, Day 0 and Day 3 conversion wells of the 20%, 10% and unmethylated control were compromised, making comparisons with longer storage points in the absence of ethanol interference difficult (FIG. 39A,B). Data series for which the Day 0 baseline could not be reliably established due to EtOH carry-over were excluded from longitudinal analysis in FIG. 42. In addition, the CpG 79-82 assay well of the 0% control sample at the 14-day storage time point showed an atypical raw data plot of unknown cause, leading to abnormally high extrapolated copy numbers (FIG. 39C). This singular data point was also excluded.

Suspected ethanol carry-over at the time of conversion appeared to be less of an issue for the methylation plate stored under frozen conditions. Day 0 sample wells generally yielded good data and allowed for longitudinal analyses of all test samples and sporadic reduction in raw data quality in individual wells appeared largely independent of storage time. One data point (10% control, 7 day storage) was excluded from quantitative analysis due to observed QC1 failure.

Despite substantial noise observed for individual samples and timepoints, the collective data set (FIG. 42) allows for limited general conclusions: 1) Based on average detected converted copy numbers, no trend of deterioration in data quality is observed after up to 14 days of refrigerated or frozen storage of the conversion plate. 2) Although sporadic suspected ethanol contamination may have acted as a confounding variable in the performed experiment, consistency of qualitative and quantitative outcomes was generally better in the data set obtained from the frozen conversion plate.

In the absence of further investigation, the current findings indicate that prolonged post-conversion storage, if necessitated by the circumstances, should occur under frozen conditions (−20° C.).

### 10. Testing of Extrinsic Interference by EtOH Carry-Over

During the bisulfite conversion process, patient sample DNA is immobilized on a spin column in a 96-well plate format. Several clean-up steps are performed by adding M-Wash buffer (Zymo Research) to each well and centrifuging the column plate. During centrifugation M-Wash buffer waste is collected in a waste plate and subsequently discarded. However, a small amount of residual M-Wash buffer may remain in the column after centrifugation to be eluted into the final converted DNA sample. Because the prepared M-Wash buffer contains ethanol—a known PCR inhibitor—it is necessary to address the potential for PCR inhibition due to ethanol carry-over from the conversion process.

To assess the effect of residual ethanol on this assay, three patient samples (MGMT_180, MGMT_082, MGMT_014) were bisulfite converted and spiked with ethanol to final concentration of 0.625% (0.125% in the ddPCR reaction), 1.25% (0.25%), 2.5% (0.5%) and 5% (1%).

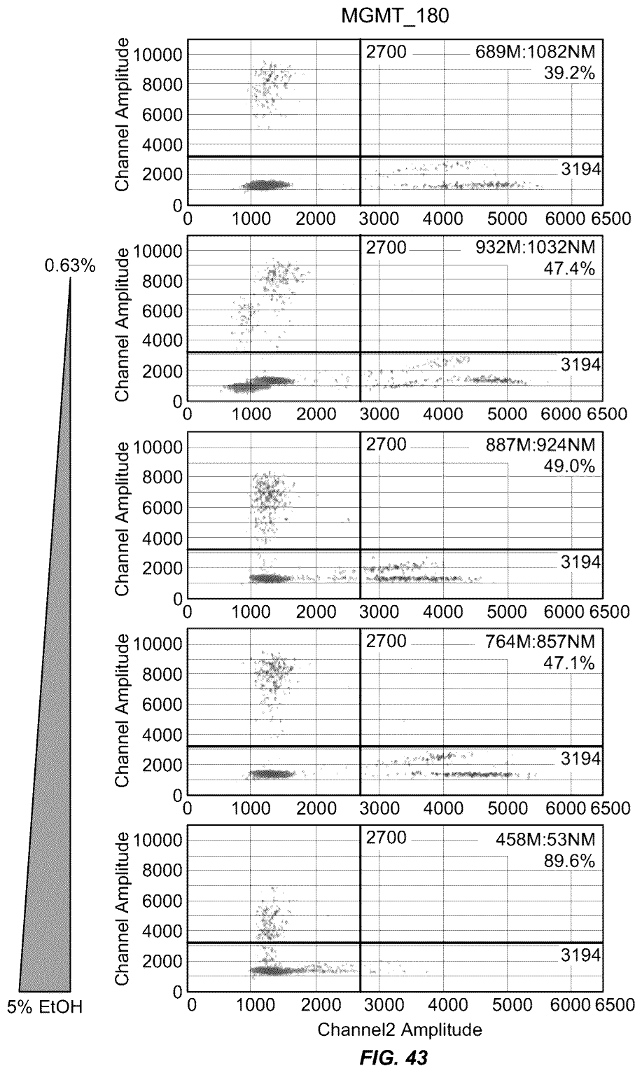

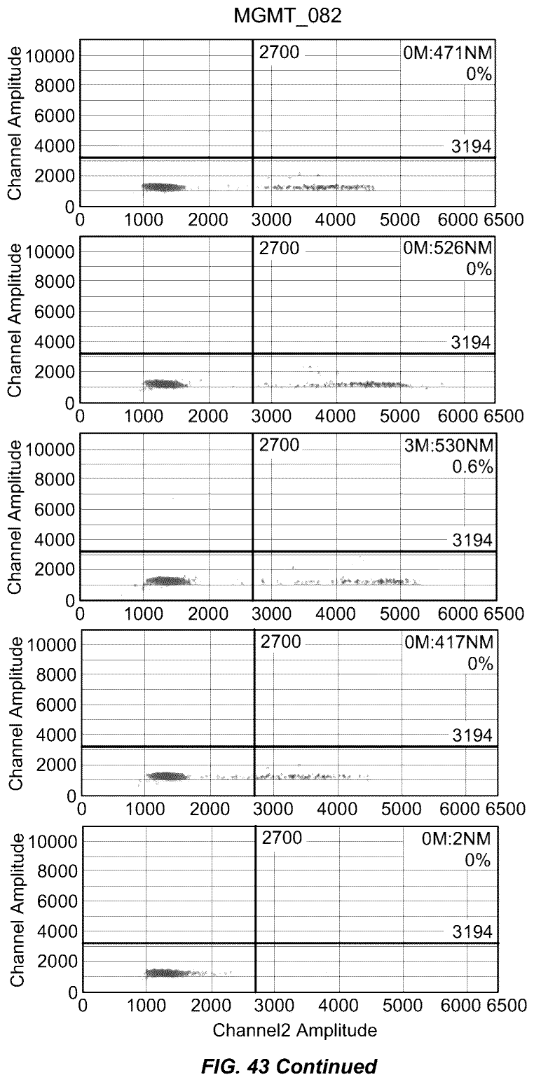

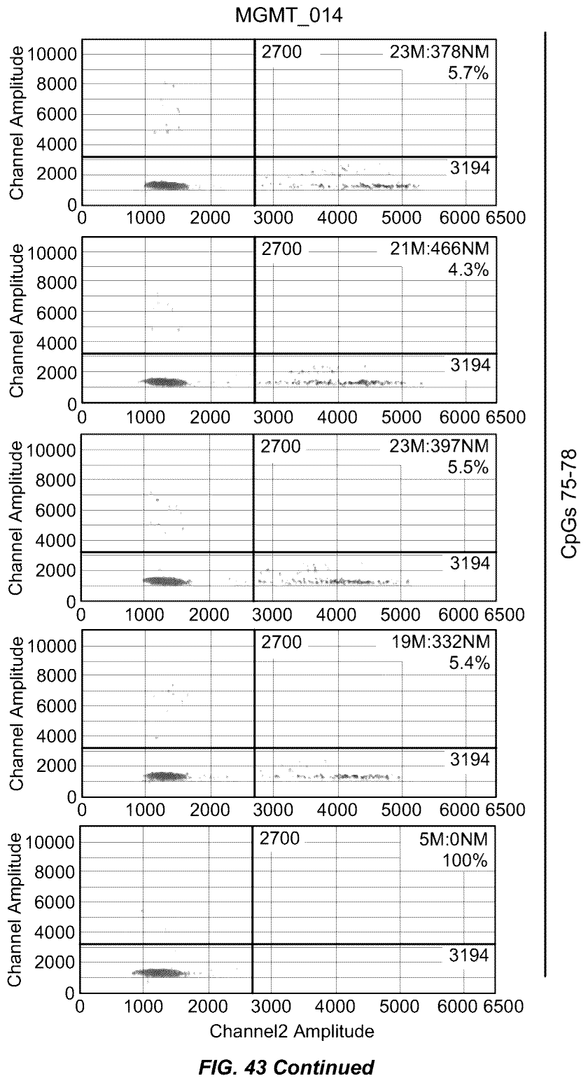

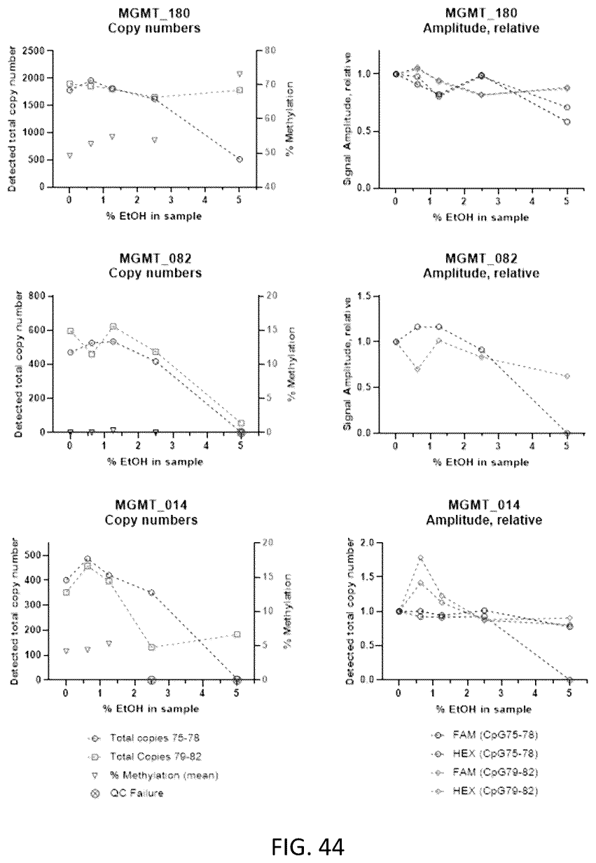

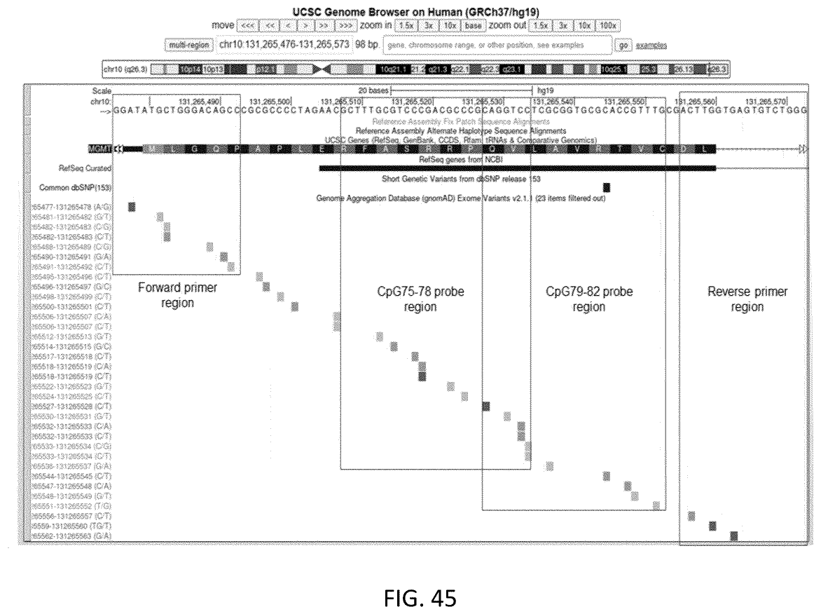

Spiked samples were tested alongside neat samples. Detected methylated and nonmethylated copy numbers, resulting mean percent methylation and mean positive signal amplitude in each detection sample was compared. Data are summarized in FIGS. 43 and 44 below.

A loss of signal amplitude was observed in the FAM and HEX detection channels with increasing contaminant ethanol contamination. This resulted in reduction of detectable total allele copy numbers and affected net observed percent methylation. Data quality was largely unaffected in the presence of up to 1.25% EtOH in the sample (0.25% final concentration in the PCR reaction). At 2.5% sample EtOH concentration one of two samples failed quality control checkpoints, at 5% two of three samples failed QC. In addition, the well 1 assay (CpGs 75-78) appeared more sensitive to the presence of ethanol contaminant and that samples with higher converted DNA concentrations were affected to a lesser extent, although generalized conclusions in these respects cannot be drawn from the small sample size.

While the data confirms that even small concentrations of ethanol (0.5% in final reaction) inhibit the ddPCR assay, the direct consequences for clinical testing are difficult to approximate. Ethanol carry-over is expected to be observed for individual reactions as an occasional consequence of the conversion protocol. The presence of ethanol after conversion is neither obvious to the operator nor can the exact concentration of ethanol be determined. Extreme cases (>1% ethanol contamination in the reaction) have increased likelihood of resulting in quality control failures and will be automatically flagged for repeat by the workbook. In addition, loss of FAM and HEX signal amplitude in individual samples in comparison with other samples and in-run controls may be evident upon visual review of the 2D data plots and such samples could be manually flagged for repeat if qualitative or quantitative outcome appear adversely affected.

### 11. Testing of Intrinsic Interference: Primer Specificity

Previously, primer specificity has been assessed using NCBI Primer-BLAST and BiSearch (36), and no problematic off-target amplicons were found. Furthermore, feasibility and development data has shown no non-specific amplification of the NTC or unconverted DNA.

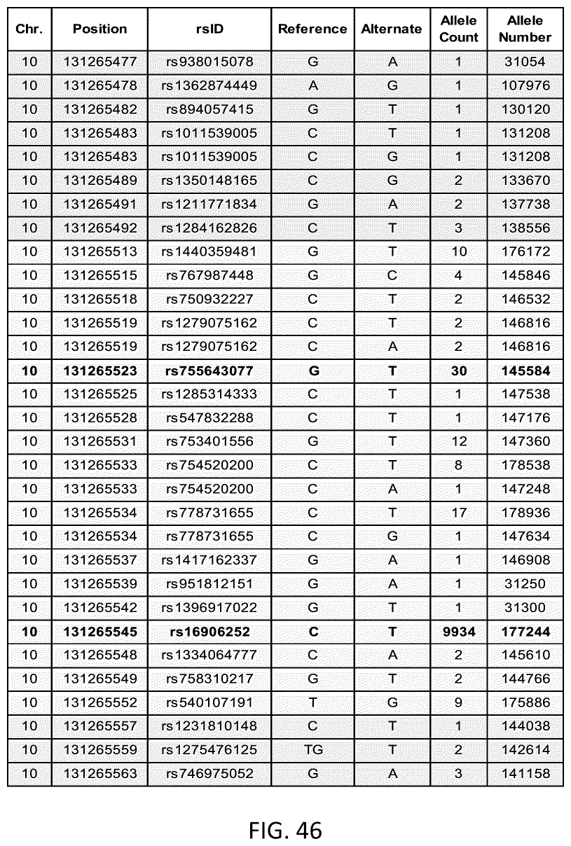

For further validation of primer specificity common single nucleotide polymorphisms (SNP, >0.1% prevalence) were searched for in the target amplicon region. The human genome browser tool at UCSC was used to examine the 98-bp amplicon region targeted by the ddPCR assay, which spans nucleotides 131265476-131265573 on chromosome 10 (GRCh37/hg19). SNPs were identified using the gnomAD 1000 Genomes databases (FIG. 45-46)

One C/T SNP, rs1690652, at position 131265545 was found to occur at a mean frequency of 5.6% across all populations. Because the alternate T allele occurs neither at a position within the primer recognition sites nor at a CpG site, no adverse effect on assay performance is expected in the population carrying the SNP. After bisulfite conversion the position will appear as a T nucleotide regardless of the original C or T base at the location.

A second SNP, a G/T polymorphism at position 131265523, occurred predominantly in South Asian populations at a frequency of 0.12%. The nucleotide changes the G position of CpG78, abolishing any methylation at the preceding cytosine. Consequences for methylation detection by the CpG75-78 probe set are unclear. All remaining SNPs in the region of interest occur at overall frequencies <0.01%.

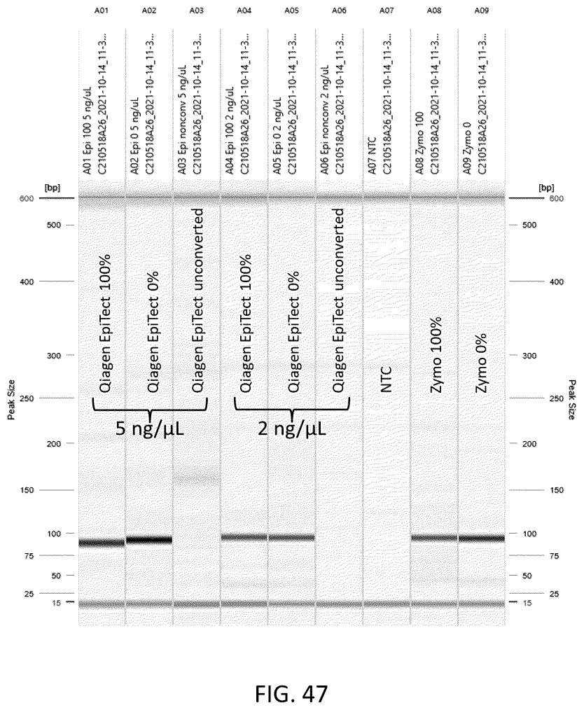

Additionally, primer specificity was verified by visualization the products of the PCR reaction on the QIAxcel (“eGene”) capillary electrophoresis platform. Qiagen EpiTect preconverted Methylated and Nonmethylated control and EpiTect unconverted control DNA were tested at 5 ng/μL and 2 ng/μL template concentration alongside aliquots of the 100%, 0% and no template converted in-run controls (Zymo) used in ddPCR testing (FIG. 47). PCR reaction components and final concentrations were identical to those used for ddPCR, except that the probe was omitted, and the droplet generation step was eliminated. PCR conditions were identical to those used for ddPCR.

PCR amplification of bisulfite converted standards resulted in a discrete band at approximately 98 bp, which is consistent with the length of the desired amplicon, regardless of methylation status. No off-target bands were observed in the no template control. Higher input (5 ng/μL template) of unconverted DNA yielded a >150 bp poorly defined band that was not observed at the lower input. Complete absence of FAM or HEX positive droplets in assay feasibility experiments using unconverted DNA as a reaction template indicates that this potentially non-specific >150 bp product has no bearing on the ddPCR assay performance.

### B. Example 2—Single-Well Multiplexing

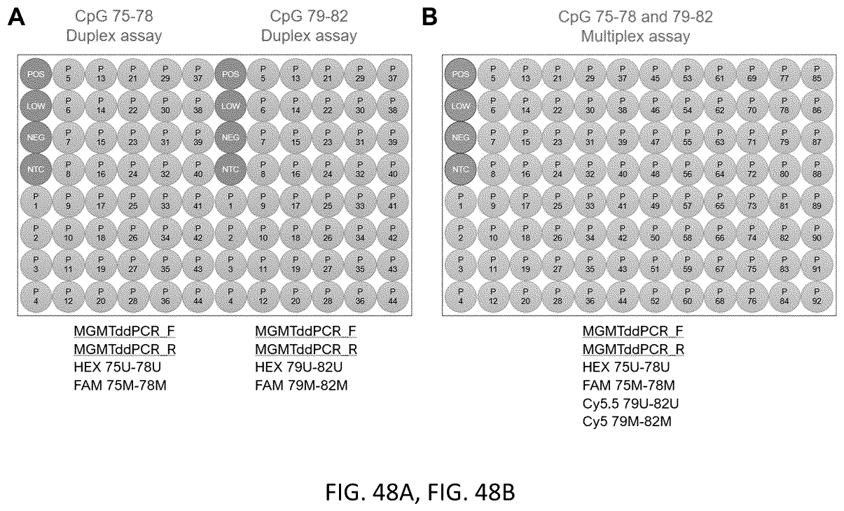

The currently validated assay design (described in Example 1) uses FAM/HEX duplex chemistry for detection of methylated and nonmethylated alleles of MGMT. Coverage of CpGs 75 to 82 requires a two-well test, with each well covering four of the eight CpGs interrogated (FIG. 48A). With multichannel ddPCR instrumentation now available, testing of all eight CpGs can be accomplished in a single test well by introduction of additional fluorophores. A Cy5.5 label was selected for the nonmethylation-specific CpG 79U-82U probe and a Cy5 label was selected for the methylation-specific CpG 79M-82M probe (FIG. 48B, Table 5). Primer sequences and probes targeting the CpG 75-78 region remained unchanged.

TABLE 5
Primer and probe sequences
for revised multiplex single-well testing.

| Oligonucleotide | |
|---|---|
| name | 5′ to 3′ sequence |
| MGMTddPCR_F | GGA TAT GTT GGG ATA GTT |
| | (SEQ ID NO: 1) |
| MGMTddPCR_R | CCC AAA CAC TCA CCA AAT |
| | (SEQ ID NO: 2) |
| HEX 75U-78U | /5HEX/AAA CCT ACA/ZEN/AAC |
| | ATC AAA ACA CAA AAC/ |
| | 3IABkFQ/ (SEQ ID NO: 3) |
| FAM 75M-78M | /56-FAM/AAC CTA CGA/ZEN/ |
| | ACG TCG AAA CGC AAA AC/ |
| | 3IABkFQ/ (SEQ ID NO: 4) |
| Cy5.5 79U-82U | /5Cy55/TAG GTT TTT GTG |
| | GTG TGT ATT GTT TG/ |
| | 3IAbRQSp/ (SEQ ID NO: 5) |
| Cy5 79M-82M | /5Cy5/TAG GTT TTC/TAO/ |
| | GCG GTG CGT ATC GTT TG/ |
| | 3IAbRQSp/ (SEQ ID NO: 6) |

Consolidating testing into a single well, has a number of advantages over two-well testing, including: 1) increased throughput by raising the maximum number of patient samples per run from 44 to 92; 2) significant cost reduction by reducing consumables and reagents needs; 3) conservation of converted test specimen by reducing required ddPCR template input (i.e. test repeats possible without repeating conversion reaction); and 4) decreased waste production by reduction of plastic consumables needs.

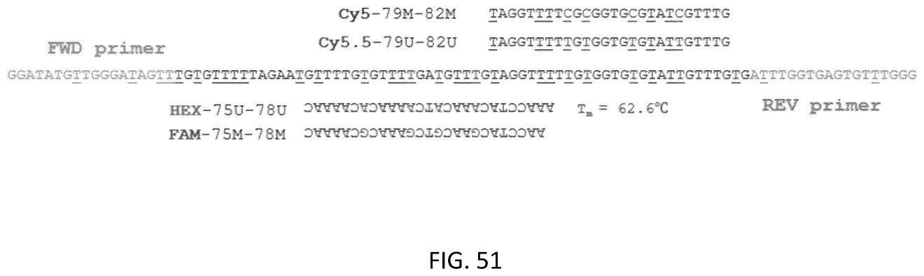

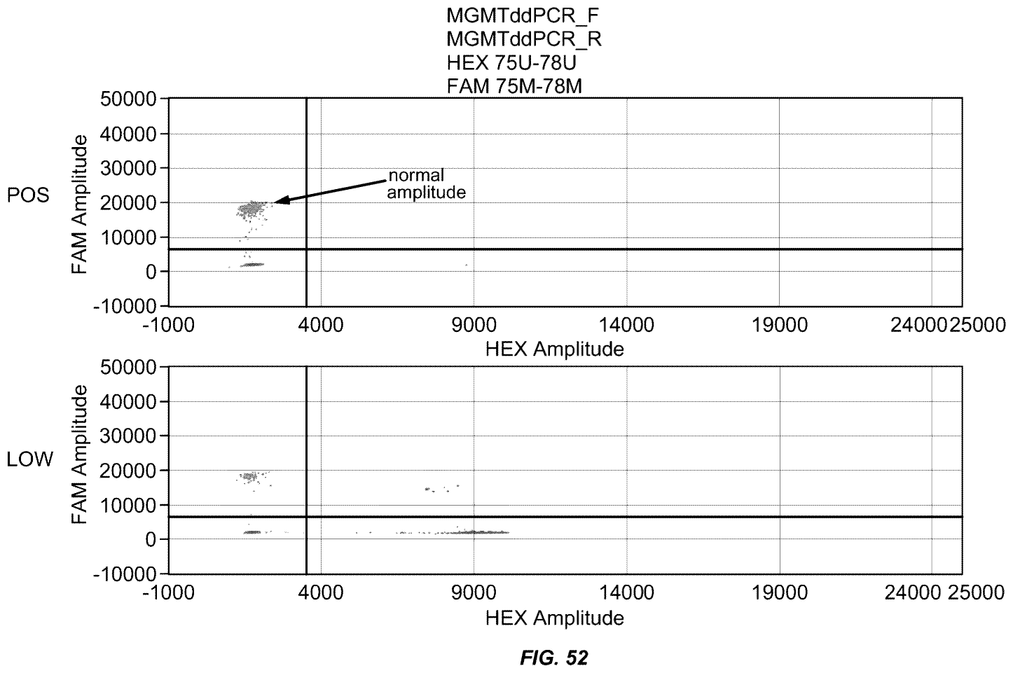

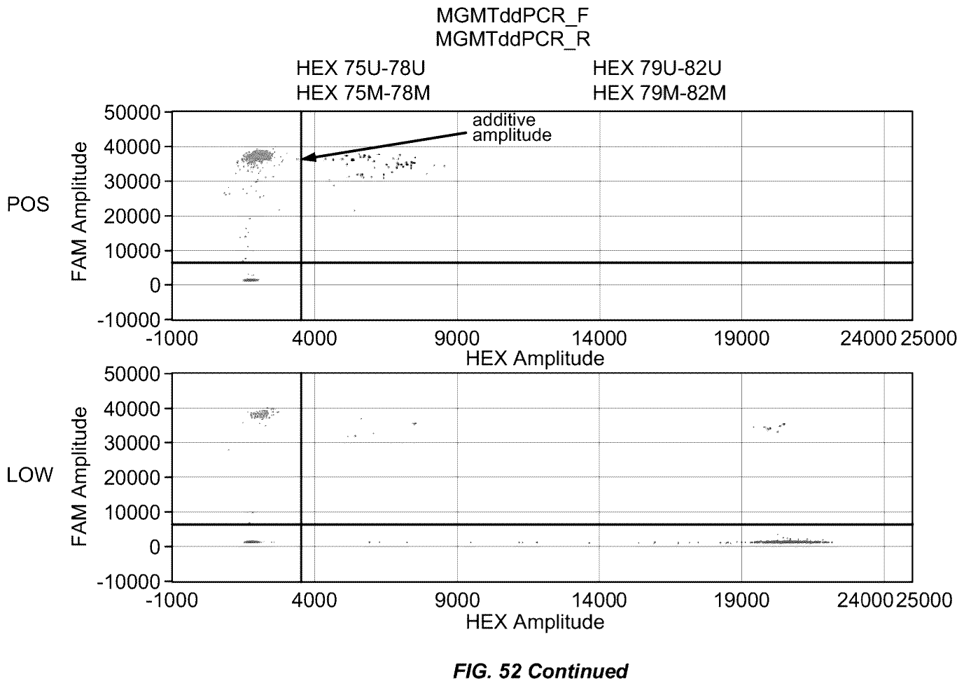

A key to avoiding assay/detection interference within the same amplicon is to design probes on opposite strands (see FIG. 51). FIG. 52 shows that FAM and HEX probes did not indicate interference (signal was additive).

Assay controls and twelve patient samples were tested with the currently validated two-well ddPCR test and a single-well test using the revised multiplex assay design. With the exception of primer and probe components, reaction composition, ddPCR protocol and general gating and data interpretation strategies were identical between both methods. Aliquots of the same conversion reactions were tested. Reaction products were read on a QX200 instrument for the two-well method and on a QX600 multichannel droplet reader for the multiplex design.

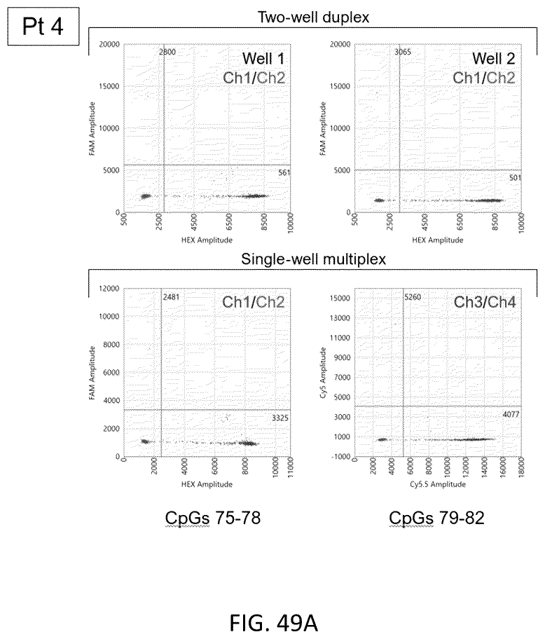

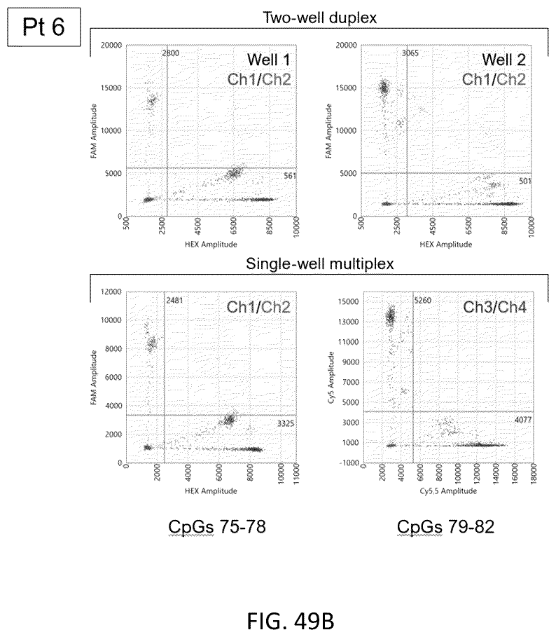

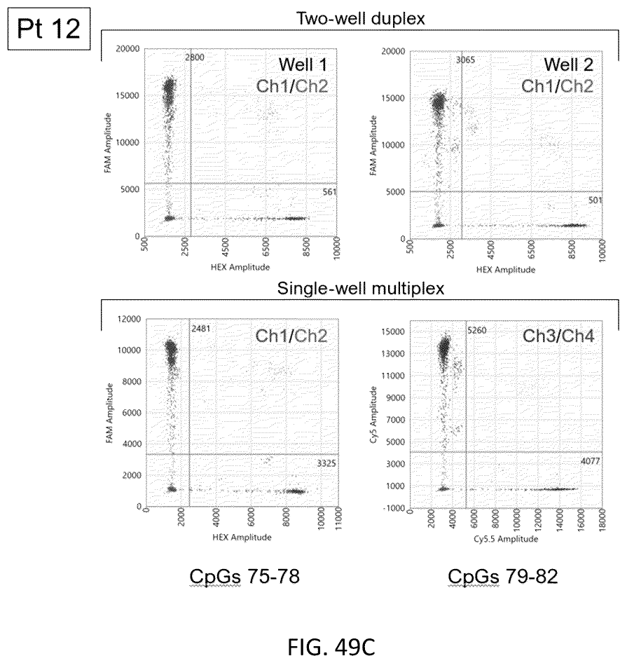

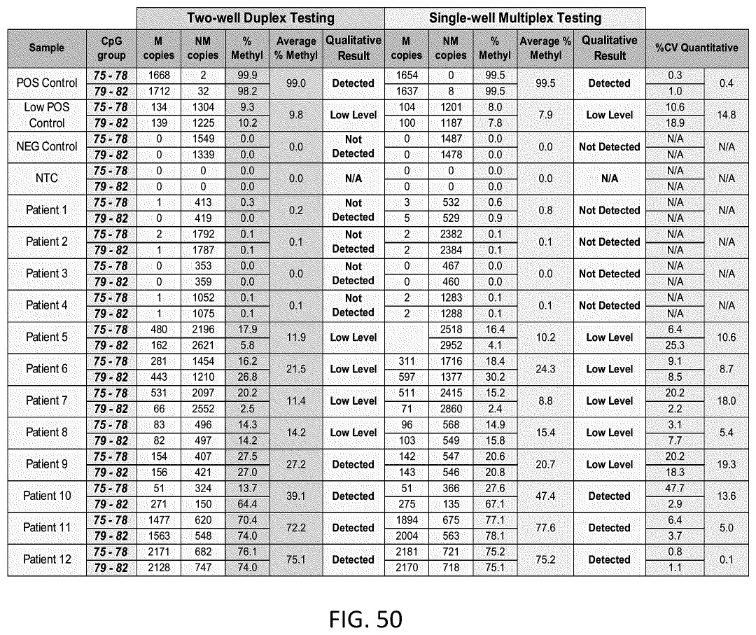

For the multiplex assay, fluorescent intensity data were collected in four detection channels: FAM, HEX, Cy5 and Cy5.5 to determine allele copy numbers for methylated CpG75-78, nonmethylated CpG75-78, methylated CpG79-82 and nonmethylated CpG79-82, respectively. Sample raw data plots for three patient samples are shown in FIG. 49A-C. A comparison of final qualitative and quantitative methylation results is included in FIG. 50.

Plot appearance, including cluster separation and sub-clustering was generally consistent between single-well and multiplex assays. Final quantitative results were identical for 11/12 patient samples. The single discrepant result was a Detected (single-well)/Low Level (multiplex) outcome in Patient sample 9, which had a quantitative clinical test result near the Low Level/Detected threshold of 25% mean methylation. Low Level/Detected discordances were also observed in replicate testing of borderline samples during validation of the two-well clinical test. Quantitative agreement of final average percent methylation (CpGs 75-82) was also good between both methods, with a median CV of 9.7% (range 0.1 to 19.3%) observed.

The single-well four-color multiplex assay design provides an alternative method for determining average percent methylation in MGMT promoter CpGs 75-82.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of determining methylation status of a target nucleic acid sequence in a sample comprising:
determining quantitative methylation data at two or more predetermined methylation sites in the target nucleic acid sequence, wherein the target nucleic acid sequence comprises a O6-methylguanine-DNA-methyltransferase (MGMT) promoter.

2. The method of claim 1, wherein determining the quantitative methylation data comprises an amplification based assay.

3. The method of claim 2, wherein the amplification based assay is droplet digital PCR (ddPCR).

4. The method of claim 3, wherein the ddPCR comprises methylation-independent conversion-dependent primers and at least two probes that span two or more CpGs of the MGMT promoter.

5. The method of claim 1, wherein the two or more predetermined methylation sites are two or more of CpGs 72-83 of the MGMT promoter.

6. The method of claim 1, wherein the two or more predetermined methylation sites are two or more of CpGs 74-82 of the MGMT promoter.

7. The method of claim 5, wherein each of the methylation sites being detected are detected in a single well or multiple wells.

8. The method of claim 1, wherein the sample is from a subject having glioma or from a tumor tissue.

9. The method of claim 1, further comprising, before determining, a step of obtaining a sample comprising the target nucleic acid sequence.

10. The method of claim 4, wherein the methylation-independent conversion-dependent primers are GGA TAT GTT GGG ATA GTT or CCC AAA CAC TCA CCA AAT.

11. The method of claim 4, wherein the at least two probes are
(SEQ ID NO: 3)
AAACCTACAAACATCAAAACACAAAAC,
(SEQ ID NO: 4)
AACCTACGAACGTCGAAACGCAAAAC,
(SEQ ID NO: 5)
TAGGTTTTTGTGGTGTGTATTGTTTG,
(SEQ ID NO: 6)
TAGGTTTTCGCGGTGCGTATCGTTTG,
(SEQ ID NO: 7)
TTTAGAACGTTTTGCGTTTCGACGTTCGTAGGT,
or
(SEQ ID NO: 8)
TTTAGAATGTTTTGTGTTTTGATGTTTGTAGGTT.

12. A method of treating a subject having cancer comprising:
determining, from a sample, quantitative methylation data at two or more predetermined methylation sites in a O6-methylguanine-DNA-methyltransferase (MGMT) promoter, wherein the sample is from the subject having cancer; and
treating the subject for cancer when the quantitative methylation data at two or more predetermined methylation sites is at or above a threshold.

13. The method of claim 12, wherein the threshold is at least 5% methylation.

14. The method of claim 12, wherein treating comprises administering an alkylating agent to the subject.

15. The method of claim 14, wherein the alkylating agent is temozolimide.

16. The method of claim 12, wherein determining the quantitative methylation data comprises an amplification based assay.

17. The method of claim 16, wherein the amplification based assay is droplet digital PCR (ddPCR).

18. The method of claim 17, wherein the ddPCR comprises methylation-independent conversion-dependent primers and at least two probes that span two or more CpGs of the MGMT promoter.

19. The method of claim 12, wherein the two or more predetermined methylation sites are two or more of CpGs 72-83 of the MGMT promoter.

20. The method of claim 12, wherein the two or more predetermined methylation sites are two or more of CpGs 75-82 of the MGMT promoter.

21. The method of claim 12, wherein each of the methylation sites being detected are detected in a single well or multiple wells.

22. The method of claim 12, wherein the sample is from a subject having glioma or from tumor tissue.

23. The method of claim 12, further comprising, before determining, a step of obtaining a sample comprising the target nucleic acid sequence.

24. The method of claim 18, wherein the methylation-independent conversion-dependent primers are GGA TAT GTT GGG ATA GTT or CCC AAA CAC TCA CCA AAT.

25. The method of claim 18, wherein the at least two probes are
(SEQ ID NO: 3)
AAACCTACAAACATCAAAACACAAAAC,
(SEQ ID NO: 4)
AACCTACGAACGTCGAAACGCAAAAC,
(SEQ ID NO: 5)
TAGGTTTTTGTGGTGTGTATTGTTTG,
(SEQ ID NO: 6)
TAGGTTTTCGCGGTGCGTATCGTTTG,
(SEQ ID NO: 7)
TTTAGAACGTTTTGCGTTTCGACGTTCGTAGGT,
or
(SEQ ID NO: 8)
TTTAGAATGTTTTGTGTTTTGATGTTTGTAGGTT.

26. The method of claim 1, further comprising treating the subject for cancer when the quantitative methylation data at two or more predetermined methylation sites is at or above a threshold.

27. The method of claim 26, wherein the threshold is at least 5% methylation.

28. The method of claim 26, wherein treating comprises administering an alkylating agent to the subject.


SEQUENCE LISTING (8 sequences)

SEQ ID NO: 1 - DNA, 18 bp, synthetic construct
  source 1..18: mol_type = other DNA
ggatatgttgggatagtt

SEQ ID NO: 2 - DNA, 18 bp, synthetic construct
  source 1..18: mol_type = other DNA
cccaaacactcaccaaat

SEQ ID NO: 3 - DNA, 27 bp, synthetic construct
  source 1..27: mol_type = other DNA
aaacctacaaacatcaaaacacaaaac

SEQ ID NO: 4 - DNA, 26 bp, synthetic construct
  source 1..26: mol_type = other DNA
aacctacgaacgtcgaaacgcaaaac

SEQ ID NO: 5 - DNA, 26 bp, synthetic construct
  source 1..26: mol_type = other DNA
taggtttttgtggtgtgtattgtttg

SEQ ID NO: 6 - DNA, 26 bp, synthetic construct
  source 1..26: mol_type = other DNA
taggttttcgcggtgcgtatcgtttg

SEQ ID NO: 7 - DNA, 33 bp, synthetic construct
  source 1..33: mol_type = other DNA
tttagaacgttttgcgtttcgacgttcgtaggt

SEQ ID NO: 8 - DNA, 34 bp, synthetic construct
  source 1..34: mol_type = other DNA
tttagaatgttttgtgttttgatgtttgtaggtt